SEQUENCE LISTING (133 sequences)

SEQ ID NO: 1 - DNA, 18 bp, synthetic construct
  source 1..18: mol_type = other DNA
cgagactactgcgagtac

SEQ ID NO: 2 - DNA, 71 bp, synthetic construct
  source 1..71: mol_type = other DNA
ggagggcagcaaacggaannnnnnnnnnnnnnnnnnnnnnnnnnnnnnnnnnncgagactactgcgagtac

SEQ ID NO: 3 - DNA, 45 bp, synthetic construct
  source 1..45: mol_type = other DNA
nnnnnnnnnnnnnnnnnnnnnnnnntagaagagtcttcctttacg

SEQ ID NO: 4 - DNA, 71 bp, synthetic construct; identical to SEQ ID NO: 2
  source 1..71: mol_type = other DNA

SEQ ID NO: 5 - DNA, 45 bp, synthetic construct; identical to SEQ ID NO: 3
  source 1..45: mol_type = other DNA

SEQ ID NO: 6 - DNA, 72 bp, synthetic construct
  source 1..72: mol_type = other DNA
cgtaaaggaagactcttcccgtttgctgccctcctcgcattctttcttgaggagggcagcaaacgggaagag

SEQ ID NO: 7 - DNA, 34 bp, synthetic construct
  source 1..34: mol_type = other DNA
cttacggatgttgcaccagcaagaaagaatgcga

SEQ ID NO: 8 - DNA, 83 bp, synthetic construct
  source 1..83: mol_type = other DNA
tcgtcggcagcgtcagatgtgtataagagacagnnnnnnnnnvvvagtatgtacgagtcnnnnnnnnngtactcgcagtagtc

SEQ ID NO: 9 - DNA, 96 bp, synthetic construct
  source 1..96: mol_type = other DNA
caagcagaagacggcatacgagatnnnnnnnncaagcagaagacggcatacgagattaaggcgagttggcaccaggcttacggatgttgcaccagc

SEQ ID NO: 10 - DNA, 90 bp, synthetic construct
  source 1..90: mol_type = other DNA
aatgatacggcgaccaccgagatctacacnnnnnnnnaatgatacggcgaccaccgagatctacacgctacgcttcgtcggcagcgtcag

SEQ ID NO: 11 - DNA, 33 bp, synthetic construct
  source 1..33: mol_type = other DNA
tcgtcggcagcgtcagatgtgtataagagacag

SEQ ID NO: 12 - DNA, 32 bp, synthetic construct
  source 1..32: mol_type = other DNA
gctggtgcaacatccgtaagcctggtgccaac

SEQ ID NO: 13 - DNA, 33 bp, synthetic construct
  source 1..33: mol_type = other DNA
ctgtctcttatacacatctgacgctgccgacga

SEQ ID NO: 14 - DNA, 32 bp, synthetic construct
  source 1..32: mol_type = other DNA
agcaagaaagaatgcgaggagggcagcaaacg

SEQ ID NO: 15 - DNA, 72 bp, synthetic construct; identical to SEQ ID NO: 6
  source 1..72: mol_type = other DNA

SEQ ID NO: 16 - DNA, 35 bp, synthetic construct
  source 1..35: mol_type = other DNA
cttacggatgttgcaccagcaagaaaaaaaaaaaa

SEQ ID NO: 17 - DNA, 20 bp, synthetic construct
  source 1..20: mol_type = other DNA
ggacaatgccacatactttg

SEQ ID NO: 18 - DNA, 20 bp, synthetic construct
  source 1..20: mol_type = other DNA
aagacccacaccatccacaa

SEQ ID NO: 19 - DNA, 20 bp, synthetic construct
  source 1..20: mol_type = other DNA
ccaatcaggcttctctgtct

SEQ ID NO: 20 - DNA, 20 bp, synthetic construct
  source 1..20: mol_type = other DNA
ttgcaggtggttcctaatat

SEQ ID NO: 21 - DNA, 20 bp, synthetic construct
  source 1..20: mol_type = other DNA
ttcattacctggataaatcc

SEQ ID NO: 22 - DNA, 20 bp, synthetic construct
  source 1..20: mol_type = other DNA
tcctgtaggtgtgaaatcca

SEQ ID NO: 23 - DNA, 20 bp, synthetic construct
  source 1..20: mol_type = other DNA
cctcaccaatgctgggtccc

SEQ ID NO: 24 - DNA, 20 bp, synthetic construct
  source 1..20: mol_type = other DNA
gcttacctggagagaggctg

SEQ ID NO: 25 - DNA, 20 bp, synthetic construct
  source 1..20: mol_type = other DNA
gaaccaagatttggtgcagg

SEQ ID NO: 26 - DNA, 20 bp, synthetic construct
  source 1..20: mol_type = other DNA
cctttaggtttaccattgct

SEQ ID NO: 27 - DNA, 20 bp, synthetic construct
  source 1..20: mol_type = other DNA
gaattaccttgggtaccaac

SEQ ID NO: 28 - DNA, 20 bp, synthetic construct
  source 1..20: mol_type = other DNA
tctttaggaaagccagccat

SEQ ID NO: 29 - DNA, 20 bp, synthetic construct
  source 1..20: mol_type = other DNA
accatcgagtgccaaatgaa

SEQ ID NO: 30 - DNA, 20 bp, synthetic construct
  source 1..20: mol_type = other DNA
ttttaaagataaatgcaagg

SEQ ID NO: 31 - DNA, 20 bp, synthetic construct
  source 1..20: mol_type = other DNA
aaattaccttataccactgt

SEQ ID NO: 32 - DNA, 20 bp, synthetic construct
  source 1..20: mol_type = other DNA
tatctaggattgcaaacctc

SEQ ID NO: 33 - DNA, 20 bp, synthetic construct
  source 1..20: mol_type = other DNA
agccttaccatgagcgtctg

SEQ ID NO: 34 - DNA, 20 bp, synthetic construct
  source 1..20: mol_type = other DNA
catttagtgactctgacagt

SEQ ID NO: 35 - DNA, 20 bp, synthetic construct
  source 1..20: mol_type = other DNA
ttcccgaagtaacaagggga

SEQ ID NO: 36 - DNA, 20 bp, synthetic construct
  source 1..20: mol_type = other DNA
cccagggaacatgaaggtct

SEQ ID NO: 37 - DNA, 20 bp, synthetic construct
  source 1..20: mol_type = other DNA
gtctgcagagcccacacgtg

SEQ ID NO: 38 - DNA, 20 bp, synthetic construct
  source 1..20: mol_type = other DNA
ctcaccatgctcgctgggct

SEQ ID NO: 39 - DNA, 20 bp, synthetic construct
  source 1..20: mol_type = other DNA
aagtccaggcacaactgctt

SEQ ID NO: 40 - DNA, 20 bp, synthetic construct
  source 1..20: mol_type = other DNA
ccttccacgtcttgggatct

SEQ ID NO: 41 - DNA, 20 bp, synthetic construct
  source 1..20: mol_type = other DNA
gtcaccaatcctgtccctag

SEQ ID NO: 42 - DNA, 20 bp, synthetic construct
  source 1..20: mol_type = other DNA
gaatttcccccaaataataa

SEQ ID NO: 43 - DNA, 20 bp, synthetic construct
  source 1..20: mol_type = other DNA
acaatccaggaaaagtggaa

SEQ ID NO: 44 - DNA, 20 bp, synthetic construct
  source 1..20: mol_type = other DNA
gaacttaccttttatactgc

SEQ ID NO: 45 - DNA, 20 bp, synthetic construct
  source 1..20: mol_type = other DNA
ttacatgtgtggggtgttac

SEQ ID NO: 46 - DNA, 20 bp, synthetic construct
  source 1..20: mol_type = other DNA
gtagtttcgggatcataatc

SEQ ID NO: 47 - DNA, 20 bp, synthetic construct
  source 1..20: mol_type = other DNA
tactcaccatctcggcatac

SEQ ID NO: 48 - DNA, 20 bp, synthetic construct
  source 1..20: mol_type = other DNA
tgacccaaagagaatttgat

SEQ ID NO: 49 - DNA, 20 bp, synthetic construct
  source 1..20: mol_type = other DNA
agaaatattacaataatgat

SEQ ID NO: 50 - DNA, 20 bp, synthetic construct
  source 1..20: mol_type = other DNA
atgcaggcatcaatccattc

SEQ ID NO: 51 - DNA, 20 bp, synthetic construct
  source 1..20: mol_type = other DNA
ccacgctctgggaaatctgc

SEQ ID NO: 52 - DNA, 20 bp, synthetic construct
  source 1..20: mol_type = other DNA
caggtctcccacaaacacat

SEQ ID NO: 53 - DNA, 20 bp, synthetic construct
  source 1..20: mol_type = other DNA
agatccgggaagagtggaac

SEQ ID NO: 54 - DNA, 20 bp, synthetic construct
  source 1..20: mol_type = other DNA
gatgcaacccctctccttga

SEQ ID NO: 55 - DNA, 20 bp, synthetic construct
  source 1..20: mol_type = other DNA
gttctcgtcatacagggcaa

SEQ ID NO: 56 - DNA, 20 bp, synthetic construct
  source 1..20: mol_type = other DNA
tgtctaccaagtgcagttta

SEQ ID NO: 57 - DNA, 20 bp, synthetic construct
  source 1..20: mol_type = other DNA
ttcacggccgacatcatgtc

SEQ ID NO: 58 - DNA, 20 bp, synthetic construct
  source 1..20: mol_type = other DNA
gatgtcggccgtgaaccatt

SEQ ID NO: 59 - DNA, 20 bp, synthetic construct
  source 1..20: mol_type = other DNA
agctcgaaggcgtagagtga

SEQ ID NO: 60 - DNA, 20 bp, synthetic construct
  source 1..20: mol_type = other DNA
cactatcggaatggtaagag

SEQ ID NO: 61 - DNA, 20 bp, synthetic construct
  source 1..20: mol_type = other DNA
accctccagagtgtactgtt

SEQ ID NO: 62 - DNA, 20 bp, synthetic construct
  source 1..20: mol_type = other DNA
cagacacatactccataatt

SEQ ID NO: 63 - DNA, 20 bp, synthetic construct
  source 1..20: mol_type = other DNA
ttatcagagaagaagacagg

SEQ ID NO: 64 - DNA, 20 bp, synthetic construct
  source 1..20: mol_type = other DNA
gtcataccggcacatctcca

SEQ ID NO: 65 - DNA, 20 bp, synthetic construct
  source 1..20: mol_type = other DNA
accacagacaacctgctccc

SEQ ID NO: 66 - DNA, 20 bp, synthetic construct
  source 1..20: mol_type = other DNA
gcagcaggaaggtccccggc

SEQ ID NO: 67 - DNA, 20 bp, synthetic construct
  source 1..20: mol_type = other DNA
aaatctatacaatataggaa

SEQ ID NO: 68 - DNA, 20 bp, synthetic construct
  source 1..20: mol_type = other DNA
atgcagggctgtctttccac

SEQ ID NO: 69 - DNA, 20 bp, synthetic construct
  source 1..20: mol_type = other DNA
tacacctacgaacatgaccc

SEQ ID NO: 70 - DNA, 20 bp, synthetic construct
  source 1..20: mol_type = other DNA
aattggaacagaaatacacc

SEQ ID NO: 71 - DNA, 20 bp, synthetic construct
  source 1..20: mol_type = other DNA
gcagcttaaaaagttggagg

SEQ ID NO: 72 - DNA, 20 bp, synthetic construct
  source 1..20: mol_type = other DNA
caagttcggcagcagcttaa

SEQ ID NO: 73 - DNA, 20 bp, synthetic construct
  source 1..20: mol_type = other DNA
gttgcggagagtctgcagca

SEQ ID NO: 74 - DNA, 20 bp, synthetic construct
  source 1..20: mol_type = other DNA
gaagatttacaagatgaata

SEQ ID NO: 75 - DNA, 20 bp, synthetic construct
  source 1..20: mol_type = other DNA
acttcagcagcttgactcaa

SEQ ID NO: 76 - DNA, 20 bp, synthetic construct
  source 1..20: mol_type = other DNA
ccagccgagatgctaagagc

SEQ ID NO: 77 - DNA, 20 bp, synthetic construct
  source 1..20: mol_type = other DNA
tcttccagatcccatggaag

SEQ ID NO: 78 - DNA, 20 bp, synthetic construct
  source 1..20: mol_type = other DNA
gccagggtctcatgcgcatc

SEQ ID NO: 79 - DNA, 20 bp, synthetic construct
  source 1..20: mol_type = other DNA
accttcagaggtggagggca

SEQ ID NO: 80 - DNA, 20 bp, synthetic construct
  source 1..20: mol_type = other DNA
tgccctcacgggccaggagg

SEQ ID NO: 81 - DNA, 20 bp, synthetic construct
  source 1..20: mol_type = other DNA
tcacctgaaggaccacttcc

SEQ ID NO: 82 - DNA, 20 bp, synthetic construct
  source 1..20: mol_type = other DNA
tgatccaagcaagcattggg

SEQ ID NO: 83 - DNA, 20 bp, synthetic construct
  source 1..20: mol_type = other DNA
ttcagccgccagactgccat

SEQ ID NO: 84 - DNA, 20 bp, synthetic construct
  source 1..20: mol_type = other DNA
tctctcccaacatgttcagc

SEQ ID NO: 85 - DNA, 20 bp, synthetic construct
  source 1..20: mol_type = other DNA
ttacagtgcctacaccaact

SEQ ID NO: 86 - DNA, 20 bp, synthetic construct
  source 1..20: mol_type = other DNA
tactcaccatctcggcatac

SEQ ID NO: 87 - DNA, 20 bp, synthetic construct
  source 1..20: mol_type = other DNA
tcttaccattccgatagtgt

SEQ ID NO: 88 - DNA, 20 bp, synthetic construct
  source 1..20: mol_type = other DNA
tttaccatctgccattgttt

SEQ ID NO: 89 - DNA, 20 bp, synthetic construct
  source 1..20: mol_type = other DNA
tactcacggcatgcctgtgc

SEQ ID NO: 90 - DNA, 20 bp, synthetic construct
  source 1..20: mol_type = other DNA
cttccaggttctatttcacc

SEQ ID NO: 91 - DNA, 20 bp, synthetic construct
  source 1..20: mol_type = other DNA
ttaccacaagctttagaagc

SEQ ID NO: 92 - DNA, 20 bp, synthetic construct
  source 1..20: mol_type = other DNA
aagtaccttattctgtagag

SEQ ID NO: 93 - DNA, 20 bp, synthetic construct
  source 1..20: mol_type = other DNA
gtcaccaatcctgtccctag

SEQ ID NO: 94 - DNA, 20 bp, synthetic construct
  source 1..20: mol_type = other DNA
ctcaccggactccagcgcca

SEQ ID NO: 95 - DNA, 20 bp, synthetic construct
  source 1..20: mol_type = other DNA
gtcaccaatcctgtccctag

SEQ ID NO: 96 - DNA, 24 bp, synthetic construct
  source 1..24: mol_type = other DNA
caagcagaagacggcatacgagat

SEQ ID NO: 97 - DNA, 29 bp, synthetic construct
  source 1..29: mol_type = other DNA
aatgatacggcgaccaccgagatctacac

SEQ ID NO: 98 - DNA, 32 bp, synthetic construct
  source 1..32: mol_type = other DNA
gttggcaccaggcttacggatgttgcaccagc

SEQ ID NO: 99 - DNA, 16 bp, synthetic construct
  source 1..16: mol_type = other DNA
tcgtcggcagcgtcag

SEQ ID NO: 100 - DNA, 64 bp, synthetic construct
  source 1..64: mol_type = other DNA
caagcagaagacggcatacgagattaaggcgagttggcaccaggcttacggatgttgcaccagc

SEQ ID NO: 101 - DNA, 64 bp, synthetic construct
  source 1..64: mol_type = other DNA
caagcagaagacggcatacgagatcgtactaggttggcaccaggcttacggatgttgcaccagc

SEQ ID NO: 102 - DNA, 64 bp, synthetic construct
  source 1..64: mol_type = other DNA
caagcagaagacggcatacgagataggcagaagttggcaccaggcttacggatgttgcaccagc

SEQ ID NO: 103 - DNA, 64 bp, synthetic construct
  source 1..64: mol_type = other DNA
caagcagaagacggcatacgagattcctgagcgttggcaccaggcttacggatgttgcaccagc

SEQ ID NO: 104 - DNA, 64 bp, synthetic construct
  source 1..64: mol_type = other DNA
caagcagaagacggcatacgagatggactcctgttggcaccaggcttacggatgttgcaccagc

SEQ ID NO: 105 - DNA, 64 bp, synthetic construct
  source 1..64: mol_type = other DNA
caagcagaagacggcatacgagattaggcatggttggcaccaggcttacggatgttgcaccagc

SEQ ID NO: 106 - DNA, 64 bp, synthetic construct
  source 1..64: mol_type = other DNA
caagcagaagacggcatacgagatctctctacgttggcaccaggcttacggatgttgcaccagc

SEQ ID NO: 107 - DNA, 64 bp, synthetic construct
  source 1..64: mol_type = other DNA
caagcagaagacggcatacgagatcagagagggttggcaccaggcttacggatgttgcaccagc

SEQ ID NO: 108 - DNA, 64 bp, synthetic construct
  source 1..64: mol_type = other DNA
caagcagaagacggcatacgagatgatttccagttggcaccaggcttacggatgttgcaccagc

SEQ ID NO: 109 - DNA, 64 bp, synthetic construct
  source 1..64: mol_type = other DNA
caagcagaagacggcatacgagatatcatgttgttggcaccaggcttacggatgttgcaccagc

SEQ ID NO: 110 - DNA, 64 bp, synthetic construct
  source 1..64: mol_type = other DNA
caagcagaagacggcatacgagattttcatcagttggcaccaggcttacggatgttgcaccagc

SEQ ID NO: 111 - DNA, 64 bp, synthetic construct
  source 1..64: mol_type = other DNA
caagcagaagacggcatacgagatagtccgacgttggcaccaggcttacggatgttgcaccagc

SEQ ID NO: 112 - DNA, 64 bp, synthetic construct
  source 1..64: mol_type = other DNA
caagcagaagacggcatacgagatgctagaaagttggcaccaggcttacggatgttgcaccagc

SEQ ID NO: 113 - DNA, 64 bp, synthetic construct
  source 1..64: mol_type = other DNA
caagcagaagacggcatacgagatcttggttagttggcaccaggcttacggatgttgcaccagc

SEQ ID NO: 114 - DNA, 64 bp, synthetic construct
  source 1..64: mol_type = other DNA
caagcagaagacggcatacgagatcgatacacgttggcaccaggcttacggatgttgcaccagc

SEQ ID NO: 115 - DNA, 64 bp, synthetic construct
  source 1..64: mol_type = other DNA
caagcagaagacggcatacgagatttgatggagttggcaccaggcttacggatgttgcaccagc

SEQ ID NO: 116 - DNA, 64 bp, synthetic construct
  source 1..64: mol_type = other DNA
caagcagaagacggcatacgagatgacatgccgttggcaccaggcttacggatgttgcaccagc

SEQ ID NO: 117 - DNA, 64 bp, synthetic construct
  source 1..64: mol_type = other DNA
caagcagaagacggcatacgagattaaagccagttggcaccaggcttacggatgttgcaccagc

SEQ ID NO: 118 - DNA, 64 bp, synthetic construct
  source 1..64: mol_type = other DNA
caagcagaagacggcatacgagatctgaattcgttggcaccaggcttacggatgttgcaccagc

SEQ ID NO: 119 - DNA, 64 bp, synthetic construct
  source 1..64: mol_type = other DNA
caagcagaagacggcatacgagatgcactatagttggcaccaggcttacggatgttgcaccagc

SEQ ID NO: 120 - DNA, 64 bp, synthetic construct
  source 1..64: mol_type = other DNA
caagcagaagacggcatacgagatggatcagcgttggcaccaggcttacggatgttgcaccagc

SEQ ID NO: 121 - DNA, 64 bp, synthetic construct
  source 1..64: mol_type = other DNA
caagcagaagacggcatacgagatttcgactcgttggcaccaggcttacggatgttgcaccagc

SEQ ID NO: 122 - DNA, 64 bp, synthetic construct
  source 1..64: mol_type = other DNA
caagcagaagacggcatacgagatggttttgggttggcaccaggcttacggatgttgcaccagc

SEQ ID NO: 123 - DNA, 64 bp, synthetic construct
  source 1..64: mol_type = other DNA
caagcagaagacggcatacgagatgtactggtgttggcaccaggcttacggatgttgcaccagc

SEQ ID NO: 124 - DNA, 53 bp, synthetic construct
  source 1..53: mol_type = other DNA
aatgatacggcgaccaccgagatctacacgctacgcttcgtcggcagcgtcag

SEQ ID NO: 125 - DNA, 53 bp, synthetic construct
  source 1..53: mol_type = other DNA
aatgatacggcgaccaccgagatctacaccgaggctgtcgtcggcagcgtcag

SEQ ID NO: 126 - DNA, 53 bp, synthetic construct
  source 1..53: mol_type = other DNA
aatgatacggcgaccaccgagatctacacaagaggcatcgtcggcagcgtcag

SEQ ID NO: 127 - DNA, 53 bp, synthetic construct
  source 1..53: mol_type = other DNA
aatgatacggcgaccaccgagatctacacgtagaggatcgtcggcagcgtcag

SEQ ID NO: 128 - DNA, 53 bp, synthetic construct
  source 1..53: mol_type = other DNA
aatgatacggcgaccaccgagatctacacgtcgtgattcgtcggcagcgtcag

SEQ ID NO: 129 - DNA, 53 bp, synthetic construct
  source 1..53: mol_type = other DNA
aatgatacggcgaccaccgagatctacacaccactgttcgtcggcagcgtcag

SEQ ID NO: 130 - DNA, 53 bp, synthetic construct
  source 1..53: mol_type = other DNA
aatgatacggcgaccaccgagatctacactggatctgtcgtcggcagcgtcag

SEQ ID NO: 131 - DNA, 53 bp, synthetic construct
  source 1..53: mol_type = other DNA
aatgatacggcgaccaccgagatctacacccgtttgttcgtcggcagcgtcag

SEQ ID NO: 132 - DNA, 96 bp, synthetic construct; identical to SEQ ID NO: 9
  source 1..96: mol_type = other DNA

SEQ ID NO: 133 - DNA, 90 bp, synthetic construct; identical to SEQ ID NO: 10
  source 1..90: mol_type = other DNA

(19) United States
(12) Patent Application Publication
(10) Pub. No.: US 2026/0117280 A1
(43) Pub. Date: Apr. 30, 2026

(54) TARGETED GENOMIC SEQUENCING IN SINGLE CELLS

(71) Applicants: THE BOARD INSTITUTE, INC., Cambridge, MA (US); PRESIDENT AND FELLOWS OF HARVARD COLLEGE, Cambridge, MA (US); THE GENERAL HOSPITAL CORPORATION, Boston, MA (US)

(72) Inventors: Fei CHEN, Cambridge, MA (US); Daniel B. GRAHAM, Cambridge, MA (US); Kai LIU, Boston, MA (US); Ramnik XAVIER, Cambridge, MA (US)

(73) Assignees: THE BROAD INSTITUTE, INC., Cambridge, MA (US); PRESIDENT AND FELLOWS OF HARVARD COLLEGE, Cambridge, MA (US); THE GENERAL HOSPITAL CORPORATION, Boston, MA (US)

(21) Appl. No.: 19/314,852

(22) Filed: Aug. 29, 2025

Related U.S. Application Data
(60) Provisional application No. 63/689,259, filed on Aug. 30, 2024.

(51) Int. Cl. C12Q 1/6806; C12Q 1/6874

(52) U.S. Cl. CPC C12Q 1/6806; C12Q 1/6874

(57) ABSTRACT

Methods and compositions capable of obtaining paired transcriptome and genotype information from single cells in high throughput are provided. Disclosed methods and compositions obtain paired transcriptome and genotype information from single cells in a manner that is highly sensitive, capable of detecting even very low-level transcripts in single cells, and associating such low-level transcripts and abundance information with associated genotypes, within a single cell.

### CROSS-REFERENCE TO RELATED APPLICATIONS

This application claims the benefit of U.S. Provisional Application No. 63/689,259, filed Aug. 30, 2024. The entire contents of the above-identified application are hereby fully incorporated by reference.

### REFERENCE TO AN ELECTRONIC SEQUENCE LISTING

The instant application contains a Sequence Listing which has been filed electronically in XML format and is hereby incorporated by reference in its entirety. Said XML file, created on Jan. 6, 2026, is named 114203-2800_SL.xml and is 120,078 Bytes in size.

### FIELD OF THE DISCLOSURE

The present disclosure relates to compositions and methods for obtaining gene expression and genotype information from single cells in a population of cells.

### BACKGROUND

Advancements in genomics have revolutionized human genetics by defining the genetic architecture of disease. However, identifying causal variants and their mechanisms of action remains a challenge in translating genetics into therapeutic interventions. Recent development of methods for single-cell RNA-sequencing (scRNA-seq) has provided the ability to study the RNA expression patterns of individual cells in a heterogeneous population. However, simultaneous measurement of multiple types of macromolecules (such as that of RNA expression and DNA sequence) in the same cell remains challenging. Thus, there is a need for high-throughput simultaneous measurements of RNA expression (particularly for transcripts of low prevalence, where highly sensitive detection is required) and DNA genotype within single cells.

### SUMMARY OF THE DISCLOSURE

The present disclosure is based, at least in part, upon discovery of methods and compositions capable of providing paired transcriptome and genotype information from single cells in high throughput, in a manner that is highly sensitive (capable of detecting even very low-level transcripts in single cells, associating such low-level transcripts and abundance information with associated genotypes, within a single cell). One approach disclosed herein, termed Sensitive Transcriptomics And Genotyping by sequencing (STAG-seq), is a high-throughput platform designed to define mechanistic genotype-phenotype relationships through simultaneous single-cell measurements of genomic DNA and RNA transcripts. Combined with base-editing, STAG-seq enables functionalization of variants in relevant cellular contexts. The applicability of this approach has now been demonstrated in several settings. First, genetic perturbations were successfully screened to identify monoallelic and biallelic variant effects in primary human macrophages treated with innate immune stimuli. Next, clinically relevant missense variants associated with immunodeficiency and autoimmunity were phenotyped. Then, a noncoding variant in a pleiotropic autoimmunity locus that governs TNRC18 expression in primary T cells was defined. The STAG-seq of the instant disclosure thus enables variant phenotyping at scale to advance functional genomics and disease biology.

In one aspect, the disclosure provides a method for generating paired barcoded gene-specific amplicons and barcoded RNA expression tracking constructs in a population of individual discrete volumes each corresponding to a single cell of a population of cells, the method involving: (i) contacting a population of cells with a first selection of oligonucleotides capable of detecting target RNA levels and selectively generating an RNA expression tracking construct in the presence of the target RNA; (ii) encapsulating individual cells of the population of cells in first individual discrete volumes, the first individual discrete volumes including cell lysis buffer and proteases, thereby generating lysed individual cells in the first individual discrete volumes; (iii) contacting the lysed individual cells in the first individual discrete volumes with a barcoding mixture, the barcoding mixture including bead-attached barcoding oligonucleotides, and genotyping probes under conditions sufficient to generate a population of lysed individual cells and individual beads having bead-attached barcoding oligonucleotides and genotyping probes in the second individual discrete volumes; (iv) incubating the population of lysed individual cells and individual beads having bead-attached barcoding oligonucleotides and genotyping probes in the second individual discrete volumes under conditions sufficient to allow for: amplification of the genotyping probes to proceed, thereby generating gene-specific amplicons in the second individual discrete volumes; and polymerase-mediated extension of the bead-attached barcoding oligonucleotides along the gene-specific amplicons and along the RNA expression tracking constructs in the second individual discrete volumes, thereby generating paired barcoded gene-specific amplicons and barcoded RNA expression tracking constructs in the second individual discrete volumes each corresponding to a single cell of the population of cells.

In one embodiment, the RNA expression tracking construct includes one or more sensing oligonucleotides and a targeting probe, where the targeting probe includes a unique molecular identifier (UMI) and a 3′-terminal sequence capable of sequence-specific hybridization to a barcoding oligonucleotide.

In another embodiment, the method further includes the steps of: (v) pooling the second individual discrete volumes, thereby forming a pooled mixture; (vi) obtaining sequence information for the barcoded gene-specific amplicons and barcoded expression tracking constructs from the pooled mixture; and (vii) identifying paired genotype information and target RNA expression levels from the sequence information by associating the UMI and barcode sequences of the single cell of the population of cells with the single cell, thereby obtaining paired genotype information and target RNA expression levels from the single cell of the population of cells.

In another aspect, the disclosure provides a method for obtaining paired target RNA expression levels and genotype information from a single cell of a population of cells, the method involving: (i) contacting a population of cells with a first selection of oligonucleotides capable of detecting target RNA levels and in the presence of the target RNA selectively generating an RNA expression tracking construct that includes one or more sensing oligonucleotides and a targeting probe having a unique molecular identifier (UMI) and a 3′-terminal sequence capable of sequence-specific hybridization to a barcoding oligonucleotide; (ii) encapsulating individual cells of the population of cells in first individual discrete volumes that include cell lysis buffer and proteases, thereby generating lysed individual cells in the first individual discrete volumes; (iii) contacting the lysed individual cells in the first individual discrete volumes with a barcoding mixture that includes bead-attached barcoding oligonucleotides and genotyping probes under conditions sufficient to generate a population of lysed individual cells and individual beads having bead-attached barcoding oligonucleotides and genotyping probes in second individual discrete volumes; (iv) incubating the population of lysed individual cells and individual beads having bead-attached barcoding oligonucleotides and genotyping probes in the second individual discrete volumes under conditions sufficient to allow for: amplification of the genotyping probes to proceed, thereby generating gene-specific amplicons in the second individual discrete volumes; and polymerase-mediated extension of the bead-attached barcoding oligonucleotides along the gene-specific amplicons and along the RNA expression tracking constructs in the second individual discrete volumes, thereby generating barcoded gene-specific amplicons and barcoded RNA expression tracking constructs in the second individual discrete volumes; (v) pooling the second individual discrete volumes, thereby forming a pooled mixture; (vi) obtaining sequence information for the barcoded gene-specific amplicons and barcoded expression tracking constructs from the pooled mixture; and (vii) identifying paired genotype information and target RNA expression levels from the sequence information by associating the UMI and barcode sequences of the single cell of the population of cells with the single cell, thereby obtaining paired genotype information and target RNA expression levels from the single cell of the population of cells.

In one embodiment, in step (i) the first selection of oligonucleotides includes two targeting probes capable of hybridizing the target RNA, whereby the targeting probes bind to a target region in the target RNA and together form an initiator sequence when both targeting probes are bound to the target RNA sequence, where the initiator sequence is not hybridized to the target region, and where the first targeting probe includes the unique molecular identifier (UMI) and the 3′-terminal sequence capable of sequence-specific hybridization to the barcoding oligonucleotide.

In a related embodiment, step (i) further includes: providing a first sensing oligonucleotide, where the first sensing oligonucleotide is a hairpin that binds to the initiator sequence, where the hairpin is opened by hybridization to the initiator to reveal a hybridization region; providing a second sensing oligonucleotide that includes a sequencing adaptor, where the second sensing oligonucleotide binds to the first sensing oligonucleotide via the hybridization region in the first sensing oligonucleotide; and attaching the second sensing oligonucleotide to the first targeting probe via ligation, thereby generating the RNA expression tracking construct that includes the second sensing oligonucleotide and the first targeting probe having the UMI and the 3′-terminal sequence capable of sequence-specific hybridization to the barcoding oligonucleotide.

Another aspect of the instant disclosure provides a method for obtaining paired target RNA expression levels and genotype information from a single cell of a population of cells, the method involving: (i) contacting a population of cells with a first selection of oligonucleotides capable of detecting target RNA levels, the first selection of oligonucleotides including two targeting probes capable of hybridizing the target RNA, whereby the targeting probes bind to a target region in the target RNA and together form an initiator sequence when both targeting probes are bound to the target RNA sequence, where the initiator sequence is not hybridized to the target region, and where the first targeting probe includes a unique molecular identifier (UMI) and a 3′-terminal sequence capable of sequence-specific hybridization to a barcoding oligonucleotide; (ii) providing a first sensing oligonucleotide, where the first sensing oligonucleotide is a hairpin that binds to the initiator sequence, where the hairpin is opened by hybridization to the initiator to reveal a hybridization region; providing a second sensing oligonucleotide having a sequencing adaptor, where the second sensing oligonucleotide binds to the first sensing oligonucleotide via the hybridization region in the first sensing oligonucleotide; and attaching the second sensing oligonucleotide to the first targeting probe via ligation, thereby generating an RNA expression tracking construct that includes the second sensing oligonucleotide and the first targeting probe including the UMI and the 3′-terminal sequence capable of sequence-specific hybridization to a barcoding oligonucleotide; (iii) encapsulating individual cells of the population of cells in first individual discrete volumes including cell lysis buffer and proteases, thereby generating lysed individual cells in the first individual discrete volumes; (iv) contacting the lysed individual cells in the first individual discrete volumes with a barcoding mixture that includes bead-attached barcoding oligonucleotides and genotyping probes under conditions sufficient to generate a population of lysed individual cells and individual beads including bead-attached barcoding oligonucleotides and genotyping probes in second individual discrete volumes; (v) incubating the population of lysed individual cells and individual beads including bead-attached barcoding oligonucleotides and genotyping probes in the second individual discrete volumes under conditions sufficient to allow for: amplification of the genotyping probes to proceed, thereby generating gene-specific amplicons in the second individual discrete volumes; and polymerase-mediated extension of the bead-attached barcoding oligonucleotides along the gene-specific amplicons and the RNA expression tracking constructs in the second individual discrete volumes, thereby generating barcoded gene-specific amplicons and barcoded RNA expression tracking constructs in the second individual discrete volumes; (vi) pooling the second individual discrete volumes, thereby forming a pooled mixture; (vii) obtaining sequence information for the barcoded gene-specific amplicons and barcoded expression tracking constructs from the pooled mixture; and (viii) identifying paired genotype information and target RNA expression levels from the sequence information by associating the UMI and barcode sequences of the single cell of the population of cells, thereby obtaining paired genotype information and target RNA expression levels from the single cell of the population of cells.

In one embodiment, the first individual discrete volumes, the second individual discrete volumes, or both, are droplets.

In another embodiment, the step of obtaining sequence information for the barcoded gene-specific amplicons and barcoded expression tracking constructs from the pooled mixture involves performance of size selection to enrich for the gene-specific amplicons and/or performance of affinity selection to enrich for the RNA expression tracking constructs.

In a related embodiment, the size selection to enrich for the gene-specific amplicons is by SPRI.

In another related embodiment, the affinity selection to enrich for the RNA expression tracking constructs involves tagging the RNA expression tracking constructs with a biotin-labeled probe and binding the tagged RNA expression tracking constructs to a streptavidin-coated solid support.

In certain embodiments, the bead-attached barcoding oligonucleotides are UV-detachable.

In some embodiments, the population of cells is fixed with methanol.

A further aspect of the disclosure provides a population of droplets, where for a majority of the droplets in the population of droplets, each droplet includes: a lysed individual cell; one or more RNA expression tracking construct(s) that include one or more sensing oligonucleotides and a targeting probe including a unique molecular identifier (UMI) and a 3′-terminal sequence capable of sequence-specific hybridization to a barcoding oligonucleotide; a barcoding mixture that includes bead-attached barcoding oligonucleotides; and genotyping probes.

In one embodiment, for a majority of the droplets in the population of droplets, each droplet further includes gene-specific amplicons produced via amplification of the genotyping probes.

In a related embodiment, for a majority of the droplets in the population of droplets, each droplet further includes barcoded gene-specific amplicons and barcoded RNA expression tracking constructs produced via polymerase-mediated extension of the bead-attached barcoding oligonucleotides along the gene-specific amplicons and the RNA expression tracking constructs.

An additional aspect of the disclosure provides a method for obtaining paired target RNA expression levels and genotype information from a single cell of a population of cells, the method involving: pooling a population of droplets of the disclosure, thereby forming a pooled mixture; obtaining sequence information for the barcoded gene-specific amplicons and barcoded expression tracking constructs from the pooled mixture; and identifying paired genotype information and target RNA expression levels from the sequence information by associating the UMI and barcode sequences of the single cell of the population of cells, thereby obtaining paired genotype information and target RNA expression levels from the single cell of the population of cells.

Another aspect of the disclosure provides a method for identifying a genomic variant that exerts a functional impact upon a cell to which the genomic variant is introduced, the method involving: (a) introducing the genomic variant to the genome of the cell; (b) obtaining by a method disclosed herein paired target RNA expression levels and genotype information for the genomic variant from single cells of a population of cells, where the single cells are confirmed by the genotype information to possess the genomic variant; and (c) identifying altered target RNA expression levels in single cells confirmed by the genotype information to possess the genomic variant, thereby identifying a genomic variant that exerts a functional impact upon the cell to which the genomic variant is introduced.

In one embodiment, the genomic variant is introduced via use of a CRISPR/Cas system.

### Definitions

Unless defined otherwise, technical and scientific terms used herein have the same meaning as commonly understood by one of ordinary skill in the art to which this disclosure pertains.

Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), multiple displacement amplification (MDA), ligation extension, or ligation chain reaction. An amplicon can be a nucleic acid molecule having a single copy of a particular nucleotide sequence (e.g. a PCR product) or multiple copies of the nucleotide sequence (e.g. a concatameric product of RCA). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, the term “attached” refers to the state of two things being joined, fastened, adhered, connected or bound to each other. For example, an analyte, such as a nucleic acid, can be attached to a material, such as a gel or solid support, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

The term “optional” or “optionally” means that the subsequent described event, circumstance or substituent may or may not occur, and that the description includes instances where the event or circumstance occurs and instances where it does not.

As used herein, the term “barcode sequence” is intended to mean a series of nucleotides in a nucleic acid that can be used to identify the nucleic acid, a characteristic of the nucleic acid (e.g., the identity and optionally the location of a bead to which the nucleic acid is attached), or a manipulation that has been carried out on the nucleic acid. The barcode sequence can be a naturally occurring sequence or a sequence that does not occur naturally in the organism from which the barcoded nucleic acid was obtained. A barcode sequence can be unique to a single nucleic acid species in a population or a barcode sequence can be shared by several different nucleic acid species in a population (e.g., all nucleic acid species attached to a single bead might possess the same barcode sequence, while different beads present a different shared barcode sequence that serves to identify each such different bead). By way of further example, each nucleic acid probe in a population can include different barcode sequences from all other nucleic acid probes in the population. Alternatively, each nucleic acid probe in a population can include different barcode sequences from some or most other nucleic acid probes in a population. For example, each probe in a population can have a barcode that is present for several different probes in the population even though the probes with the common barcode differ from each other at other sequence regions along their length. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen.

As used herein “endogenous” refers to any material from or produced inside an organism, cell, tissue or system.

As used herein, the term “exogenous” refers to any material introduced from or produced outside an organism, cell, tissue, or system.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decreases production. Molecules (e.g., transcription factors) that are involved in up-regulation or down-regulation are often called “activators” and “repressors,” respectively.

As used herein, the term “extend,” when used in reference to a nucleic acid, is intended to mean the addition of at least one nucleotide or oligonucleotide to the nucleic acid. In particular embodiments, one or more nucleotides can be added to the 3′ end of a nucleic acid, for example, via polymerase catalysis (e.g., DNA polymerase, RNA polymerase or reverse transcriptase).

Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g., ligase catalysis) methods. A nucleic acid can be extended in a template directed manner, whereby the product of extension is complementary to a template nucleic acid that is hybridized to the nucleic acid that is extended.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reactions that occur within a natural environment.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogsteen binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The terms “subject,” “individual,” and “patient” are used interchangeably herein to refer to a vertebrate, preferably a mammal, and more preferably a human. Mammals include, but are not limited to, murines, simians, humans, farm animals, sport animals, and pets. Tissues, cells and their progeny of a biological entity obtained in vivo or cultured in vitro are also encompassed.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (Ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics”, J Genet Genomics, 201, 38(3): 95-109.

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence.

As used herein, the term “poly T or poly A,” when used in reference to a nucleic acid sequence, is intended to mean a series of two or more thiamine (T) or adenine (A) bases, respectively. A poly T or poly A can include at least about 2, 5, 8, 10, 12, 15, 18, 20 or more of the T or A bases, respectively. Alternatively or additionally, a poly T or poly A can include at most about 30, 20, 18, 15, 12, 10, 8, 5 or 2 of the T or A bases, respectively.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “each,” when used in reference to a collection of items, is intended to identify an individual item in the collection but does not necessarily refer to every item in the collection. Exceptions can occur if explicit disclosure or context clearly dictates otherwise.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, non-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed embodiments presented in the disclosure.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the disclosure solely to the specific embodiments described, may best be understood in conjunction with the accompanying drawings.

FIGS. 1A to 1K show that the “Sensitive Transcriptomics And Genotyping by sequencing (STAG-seq)” approach of the disclosure enabled single-cell genotyping and parallel transcriptome profiling in high-throughput with sensitivity. FIG. 1A shows a schematic of the instant STAG-seq workflow. Fixed cells were first hybridized with probes that recognized targeted RNA molecules, then incorporated into droplets with lysis buffer (obtained from the Tapestri kit; lysis buffers often contain salts to create an ionic strength in a solution—common salts used in lysis buffers include NaCl, KCl, and (NH4)2SO4, usually at concentrations between 50 and 150 mNM) containing proteases (e.g., proteinase K) to release genomic DNA from chromatin. Subsequently, the first droplet was incorporated with the Barcoding PCR mix and Barcoding beads into the secondary droplet. After UV cleavage of barcoding oligos, the gDNA amplicons and RNA probes were amplified and sequenced. FIG. 1B shows detection of single nucleotide variants (SNVs) from THP1 and Jurkat cells pooled into a heterogeneous population. Cells were hybridized with 2,614 probes against 1,297 genes and then processed into the STAG-seq pipeline with the detection of 109 genomic regions. The called genomic variants were compared with the somatic mutations of each cell type identified by DepMap Omics (FIG. 6). Y-axis: mean VAF (Variant allele frequency) of THP1 unique alleles (17 homozygous, 2 heterozygous). X-axis: mean VAF (Variant allele frequency) of Jurkat unique alleles (8 homozygous, 12 heterozygous). The contour line shows the cell density across VAF values. Jurkat and THP1 cells were clearly separable by genotype and showed a very low mixing rate. FIG. 1C shows the distribution of cells at selected loci. Cells in FIG. 1B were projected for each SNV. The top four rows are unique homozygous and heterozygous alleles in Jurkat cells and the bottom four are unique homozygous and heterozygous alleles in THP1 cells. For loci that were WT-homozygous differing between THP1 and Jurkat cells, the VAFs of mixed cells were distributed at ˜50%. For WT-heterozygous loci, mixed cell VAFs were distributed at ˜25%. Cells were color-coded based on the identity defined in FIG. 1B. Genotyping accuracy was high at homozygous and heterozygous loci. FIG. 1D shows the fractions of genotype that were determined by the GATK variant calling for heterozygous SNPs in both THP1 and Jurkat cells. FIG. 1E shows a box plot depicting results obtained for MDMs stimulated with LPS or left untreated (PBS) and subjected to STAG-seq with the 50k transcriptome panel and 121-locus gDNA panel. The plot shows the percentage of high-confidence DNA amplicons in each cell (n=8,969 and 9,723) for the PBS and LPS samples, respectively. FIG. 1F shows the percentage of cells (n=8,969 and 9,723) that were confidently genotyped for each locus (≥10 reads) for the PBS and LPS samples. The dashed box indicates amplicons that were confidently detected in <50% cells. FIG. 1G shows fractions of genotypes determined by the GATK variant calling for the germline heterozygous SNPs presented in the DNA panel of the PBS sample. Red: GATK called as reference; light blue: GATK called as heterozygous variants; Dark blue: GATK called as homozygous variants. FIG. 1H shows box plots comparing the distribution of total UMIs per cell between STAG-seq and 10× for indicated treatments. FIG. 1I shows correlation of mean CPM/gene between bulk RNA-seq and STAG-seq of 17,885 genes in PBS-treated samples. FIG. 1J shows correlation of mean CPM/gene between 10× and bulk RNA-seq of 17,885 genes in PBS-treated samples. FIG. 1K shows correlation of expression changes of 17,885 genes (LPS vs PBS) between bulk RNA-seq and STAG-seq.

FIGS. 2A to 2H show that the STAG-seq process of the instant disclosure deciphered the function of single base changes in a matrixed base editor screen. FIG. 2A shows a schematic workflow of the matrix screen with 14 splice-site or nonsense mutations of 8 genes under 7 stimulation conditions. The red box in the matrix signifies expected variant responses with the given stimuli. FIG. 2B shows an evaluation of base editor precision and efficiency using multiple combinations of CAS9-cytosine deaminase enzymes with a control guide RNA targeting a locus with NRY PAMs. Editing efficiency at each position was calculated by NGS. FIG. 2C depicts a UMAP plot showing STAG-seq transcriptome with 1,277 genes in 56,783 cells. Edited MDMs were stimulated with the indicated TLR ligands or cytokines (see Example 1). FIG. 2D depicts heatmaps showing the standardized mean expression of marker genes (X-axis) in the unedited cells of two donors after indicated stimulation (Y-axis). The columns of the heatmap represent individual genes with mean expression (indicated by color intensity) scaled between 0 and 1 by subtracting the minimum mean expression across simulations and donors and dividing by the maximum. FIG. 2E depicts heat maps showing the effect of variant alleles (columns) on gene expression (rows) in cis at steady state (rows are Z-scored). The edited variants targeted: SPA, splice acceptor; SPD, splice donor; PTC, pre-termination codon. Het, heterozygous; Hom, homozygous. Base changes are indicated after the colon. Color intensity indicates z-scores of normalized mean expression of each gene. FIG. 2F depicts a bubble pie plot showing the specificity of variant allele effects after stimulation. Bubble size indicates percentage of stimulation-induced differentially expressed genes (DEGs) in unedited cells observed as DEGs in variant vs AAVS under the same stimulus. For stimulation DEGs, adjusted P<0.0001, log FC >0.5 or <−0.5. For variant DEGs, adjusted P<0.05. Het, heterozygous; Hom, homozygous. Base changes indicated after the colon. Red box indicates expected responses. FIG. 2G depicts quadrant plots showing variant allele effects on expression of all measured genes under indicated stimulations. Y-axis: Log FC of gene expression in unedited cells in response to stimulations. X-axis: Log FC of gene expression between variant cells and AAVS-edited cells under indicated stimulations in Y-axis. Red dots indicate genes with significant changes in variant cells. Dot size, −Log 10 of the adjusted P value. Dashed regression line indicates the trend and extent of how variants affect the simulation responses. “n” indicates cell number of each variant genotype. FIG. 2H depicts a heatmap showing the Pearson correlation coefficient of variant allele effects on the relevant stimulation responsive genes (Log FC>0.5 or −0.5<, adjustP<1-e30) between two donors. IRF1 variants under IFNγ stimulation; TLR4 and MyD88 variants under LPS stimulation; TGFBR1 variants under TGFβ stimulation; IL10RA variants under IL-10 stimulation; IL1RA variants under IL-1β stimulation. Color intensity indicates Pearson correlation coefficient of the mean log FC of stimulation responsive genes between variants and AAVS edits for both donors. Het, heterozygous; Hom, homozygous. Dashed box indicates groups of variants expected to affect the same programs.

FIGS. 3A to 3M show that STAG-seq revealed the function of clinically-relevant coding variants in the IFNγ pathway. FIG. 3A shows a schematic of the IFNγ pathway. The number of variants that were investigated in MDMs for each gene is listed in parentheses. Variants were selected from OMIM and ClinVar based on associations with immune-mediated diseases or cancer. FIG. 3B shows precision and efficiency of editing after variant calling from STAG-seq. Red and purple show cells containing only pure homozygous or heterozygous variants, respectively, without any other bystander mutations within the 20 bp window encompassing the targeted variant. Blue and green shows cells containing homozygous or heterozygous variants, respectively, along with any other mutations (homozygous or heterozygous) within the 20 bp target window. FIG. 3C depicts a UMAP plot showing STAG-seq transcriptome of 13,749 unedited cells with detection of 1,277 genes under IFN-γ stimulation. MDMs were stimulated with PBS or IFN-γ (10 ng/ml) for 6 hours. Cells were fixed and hybridized with 2,740 probes for 1,277 genes and then processed into the STAG-seq pipeline with detection of 85 genomic regions. FIG. 3D depicts quadrant plots showing the effect of two homozygous IFN-γ receptor variants on all measured genes in response to IFN-γ. Y-axis: Log FC of gene expression in unedited cells in response to IFN-γ. X-axis: Log FC of gene expression between variant cells and AAVS-edited cells under IFN-γ stimulation. Red dots indicate genes with significant changes in variant cells. Dot size, log 100 of adjusted P value. “n” indicates the cell number of each variant genotype. FIG. 3E shows a volcano plot of IFN-γ response genes in unedited MDMs. Dashed line indicates the threshold used to select genes (top 100 upregulated and downregulated genes with adjusted P<e30) for calculating gene program scores. FIG. 3F shows the effect of clinical variants W11R and R129X on the IFN-γ program. X-axis: domain structure of targeted protein and the amino acid position of each variant. Y-axis: the IFN-γ program score representing changes in IFN-γ response genes between a given variant and control cells (see Example 1). Positive scores represent an increased response to IFN-γ in edited cells (gain-of-function) while negative scores represent a decreased response to IFN-γ in edited cells (loss-of-function). Purple and blue dots represent pure homozygous or heterozygous respectively. FIGS. 3G-3K show the effect of indicated clinical variants W11R and R129X on the IFN-γ program. X-axis: domain structure of targeted protein and the amino acid position of each variant. Y-axis: the IFN-γ program score representing changes in IFN-γ response genes between a given variant and control cells (see Example 1). Positive scores represent an increased response to IFN-γ in edited cells (gain-of-function) while negative scores represent a decreased response to IFN-γ in edited cells (loss-of-function). Purple and blue dots represent pure homozygous or heterozygous respectively. FIG. 3L shows the Pearson correlation of the variant allele effects on the IFN-γ program scores in two donors. Each dot represents a variant. P(two-tailed)<0.0001. FIG. 3M depicts quadrant plots showing the effect of STAT1 variants on all measured genes in response to IFN-γ. “n” indicates the cell number of each variant genotype.

FIGS. 4A to 4H show that STAG-seq identified cis- and trans-effects of a pleiotropic variant linked to autoimmunity. FIG. 4A depicts a forest plot showing associations of rs748670681 with different autoimmune diseases (Y-axis) in FinnGen R10. Error bar represents standard error of effect size β (log odds ratio). AS, ankylosing spondylitis; CD, Crohn's disease; IBD, inflammatory bowel disease; UC, ulcerative colitis; AIHT, autoimmune hypothyroidism; TlD, type I diabetes; MS, multiple sclerosis; GD, Grave's disease. FIG. 4B shows a Manhattan plot of IBD GWAS data. Fine-mapping identified rs748670681 as an IBD risk factor (posterior inclusion probability [PIP]=1.0). Color corresponds to a r2 value to the lead variant rs748670681. FIG. 4C shows mapping of cell type specific gene regulation by rs748670681. Dot plot shows the expression changes of genes within the locus in different cell types with the rs748670681 homozygous variant relative to AAVS-edited cells. FIG. 4D shows a box plot depicting the cis-regulatory effect of rs748670681 on TNRC18 expression in CD4, CD8, and Th1 cells. FIG. 4E depicts a volcano plot illustrating the trans-regulatory impact of rs748670681 homozygous variant determined by STAG-seq. Red dots: highlighted upregulated genes; green dots: highlighted down regulated genes. FIG. 4F shows a UMAP plot showing 4,354 Th1 cells segregated into two distinct clusters with STAG-seq detection of 2,315 genes. FIG. 4G shows a dot plot displaying differentially expressed genes across the clusters identified in FIG. 4F. FIG. 4H shows a cluster composition plot comparing three indicated genotypes. The dash line indicates the proportion of cell clusters in AAVS control.

FIG. 5 shows a detailed schematic of STAG-seq library generation. The first step of STAG-seq involves hybridizing a probe pool to fixed cells. The 5′ probes are phosphorylated at the 5′ end, contain a hairpin binding sequence (gray), a specific RNA binding sequence (25 bp, red), a unique molecular identifier (UMI, yellow) and a complement sequence (blue) that anneals with the Tapestri barcoding oligo. The 3′ probes contain a hairpin binding sequence (gray) and a specific RNA binding sequence (25 bp, red). Together, the 5′ and 3′ pairs of initiator probes recognize a specific target site (52 bp) on RNA, such that each probe anneals adjacent to the other to create an initiator sequence. Then the H1B1 hairpin is added to stabilize the probe-RNA structure. Finally, the readout oligo (brown) is annealed and ligated to the 5′ probes, increasing detection specificity while providing a handle for subsequent biotin pull-down and library PCR. In the second step, the hybridized cells are distributed into the 1st emulsion containing proteases to release the genomic DNA and probes, then incorporated into the 2^{nd} emulsion with barcoding beads, oligo pools and PCR reagents. After encapsulation, the region of interest in genomic DNA is amplified exponentially and the probes are amplified linearly. In the last step, gDNA and probe libraries are extracted from the emulsion, purified and subjected to a final amplification for sequencing (see Example 1).

FIG. 6 shows a list of somatic variants in THP1 and Jurkat cell lines identified by DepMap Omics.

FIG. 7 shows a list of ClinVar variants in the key components of the IFN-γ pathway.

FIGS. 8A-8C show a list of variants, with guide RNA sequence and base editors. FIG. 8A shows the list of variants, with guide RNA sequence and base editors used in FIG. 2 (SEQ ID NOs: 17-41 are shown). FIG. 8B shows the list of variants, with guide RNA sequence and base editors used in FIG. 3 (SEQ ID NOs: 42-93 are shown). FIG. 8C shows the list of variants, with guide RNA sequence and base editors used in FIG. 4 (SEQ ID NOs: 94 and 95 are shown).

FIGS. 9A and 9B show index primers for gDNA library and probe library PCR. FIG. 9A shows index primers for gDNA library and probe library PCR for HyPR (Index Seq for P7I1-24 is SEQ ID NO: 96; Index Seq for P5I1.1-8 is SEQ ID NO: 97; Readout Seq for P711-24 is SEQ ID NO: 98; Readout Seq for P5I1.1-8 is SEQ ID NO: 99; full length sequences are SEQ ID NOs: 100-131, as well as full sequences SEQ ID NOs: 132 and 133). FIG. 9B shows index primers for gDNA library and probe library PCR for Tapestri.

FIG. 10 shows the mean variant impacts on the IFN-γ program and statistical significance.

### DETAILED DESCRIPTION OF THE DISCLOSURE

The current disclosure is based, at least in part, upon the discovery of improved methods for obtaining paired sequences for both targeted expressed RNAs and targeted genotypes (genomic DNA) in the same single cells, while simultaneously capturing such single cell information across a population of cells. The currently disclosed processes specifically address technical shortcomings in the field of functional genomics (e.g., difficulties when using existing processes in balancing throughput, recovery rates, and accuracy) by enabling simultaneous genotyping and phenotyping at the single cell level, even for transcripts that are only weakly expressed. The methods can detect somatic mutations, inherited germline variants, or engineered variants installed by genome editing to discern their respective impacts on gene expression. First, targeted gene expression is achieved using a variation of a probe-based method for RNA quantification (termed “HyPR-seq”) (Marshall et al. PNAS USA 117: 33404-413; see also U.S. Pat. No. 11,414,701). In the current disclosure, expression tracking probes have been re-engineered to be compatible with targeted genomic DNA sequencing on a specific high-throughput single cell platform (the Tapestri platform developed by Mission Bio). While the existing single cell platform has primarily been used as a means for encapsulating cells with barcoding beads in droplets, it is contemplated herein that the process of cell and barcoded bead encapsulation in droplets could alternatively be achieved with other instrumentation. The currently disclosed process (termed “STAG-seq”, for “Sensitive Transcriptomics And Genotyping by sequencing”) therefore provides a modular platform for paired genotyping and phenotyping at scale. STAG-seq allows for the examination of up to 15K cells in a single reaction, with a cell recovery rate of approximately 10%. A notable advancement is in genotyping accuracy. STAG-seq achieves an average allelic dropout rate of only 2.5%, which is a significant improvement over conventional genotyping methods (e.g., genotyping of targeted loci (GoT) or genotyping of targeted loci with single-cell chromatin accessibility (GoT-ChA)).

STAG-seq, as disclosed herein, has been developed, at least in part, as a high-throughput platform designed to define mechanistic genotype-phenotype relationships through simultaneous single-cell measurements of genomic DNA and RNA transcripts. STAG-seq has been deployed herein with base editor screens to dissect the interferon pathway and delineate a phenotypic spectrum of STAT1 missense variants associated with immunodeficiency and autoimmunity. Moreover, a pleiotropic autoimmunity locus is interrogated herein, and a noncoding variant in a regulatory element governing TNRC18 expression in myeloid cells was identified. The STAG-seq process of the disclosure enables variant phenotyping at scale to advance functional genomics and disease biology.

In some embodiments, the methods herein combine hybridization of DNA probes with a high throughput sequencing readout for digital expression quantification. The probe design can allow for detection of about 200 or more chosen transcripts including mRNAs, non-polyadenylated transcripts, and synthetic RNA barcodes, and provides high sensitivity (capabilities to detect weakly expressed transcripts) and detection efficiency. The methods and systems may be used for quantifying targeted changes in RNA expression, in association with same-cell genotyping, for use with, e.g., single-cell diagnostics, characterization of genetic variants, and potentially screens (e.g., CRISPR screens,).

In some examples, the methods may include: binding a targeting probe to a target nucleic acid; binding a first sensing oligo to the targeting probe; binding a second sensing oligo to the first sensing oligo, which brings the second sensing oligo close to the targeting probe; and ligating the second sensing oligo and the targeting probe to form an RNA expression tracking construct. The RNA expression tracking construct may be subsequently barcoded and ultimately sequenced, and the reads may be used for analyzing the target RNA. In some cases, two targeting probes may be used. Such two targeting probes may bind to adjacent target regions in the target nucleic acid.

Understanding the functional impact of DNA variation is a central mission of genetics. While human genetics has rapidly enabled discovery of genetic variation associated with a broad range of diseases, there remains a significant bottleneck in translating these discoveries into identification of causal variants and their mechanisms of action. This is due, in large part, to the need to characterize variant effects within relevant primary cell type contexts, as variant effects are highly context-dependent, influenced by cell type, cell state, and the cellular environment. This complexity often confounds interindividual comparisons in translational studies due to differences in genetic backgrounds and environmental factors (GTEx Consortium. Science, 369: 1318-1330; Kang et al. Annu. Rev. Genomics Hum. Genet., 24: 277-303; Yazar et al. Science, 376: eabf3041). To address these challenges, experimental tools that enable the generation of genetic variants within an isogenic background are essential, as they minimize confounding variables and allow for precise comparisons between variants and controls in relevant cellular contexts. Functional genomics has been revolutionized by high-throughput methods for functional characterization of genetic variants, as well as by genome editing technologies. Deep mutational scanning (DMS) and massively parallel reporter assays (MPRAs) facilitate the simultaneous analysis of a large number of variants, but these approaches are limited in their ability to replicate true allele dosage and endogenous genomic contexts, thereby hindering precise functional characterization of variants (Tabet et al. Annu. Rev. Genet., 56: 441-465; Beltran et al. Nature, 637: 885-894; Agarwal et al. Nature, 639: 411-420; Gordon et al. Nat. Protoc., 15: 2387-2412). CRISPR-based technologies, including base editing and prime editing, enable genetic alterations within the natural genome (Huang et al. Nat. Protoc., 16: 1089-1128; Chen et al. Nat. Rev. Genet., 24: 161-177; Yan et al. Nature, 628: 639-647). When integrated with single-cell methods like Perturb-seq, these tools facilitate systematic investigations of variant functions with high-content RNA readouts in pooled screens (Martin-Rufino et al. Cell, 186: 2456-2474.e24; Morris et al. Science, 380: eadh7699). However, current variant editing tools are often constrained by genomic sequence context, incomplete editing efficiency, and bystander mutations. These limitations obscure the precise determination of edited variants in individual cells, particularly when relying on gRNA proxies and in primary cell contexts where editing efficiencies are low, thereby complicating the accurate interpretation of variant effects. Direct genotyping of variants from the endogenous genome combined with high-sensitivity profiling of the transcriptome within the same single cell—which STAG-seq provides—is therefore advantageous for overcoming current limitations for variant functionalization. Plate-based methods capable of measuring both DNA and RNA are constrained by low throughput, making them impractical for large-scale studies (Rodriguez-Meira et al. Mol. Cell, 73: 1292-1305; Zachariadis et al. Mol. Cell, 80: 541-553). Microfluidic or split-pool approaches that genotype from open chromatin fragments or transcripts, while scalable, often suffer from high allele dropout rates and may fail to access key coding or noncoding genomic regions, limiting their utility for high-throughput screening (Izzo et al. Nature, 629: 1149-1157; Olsen et al. Nat. Methods, 22: 477-487). Additionally, variants associated with recessive diseases can exert significant phenotypic effects even in a heterozygous state, albeit with subtler signals (Heyne et al. Nature, 613: 519-525). Detecting effects of these variants on cellular functions requires methods such as the instantly disclosed STAG-seq process, that possess allelic sensitivity in genotyping and enhanced power to capture minor transcriptomic changes. The disclosed STAG-seq droplet-based single-cell method was designed to address existing functional genomics challenges. STAG-seq enables the sequencing of hundreds of targeted genomic DNA loci to ascertain genotypes while concurrently profiling the transcriptome with high sensitivity using probe-based detection within the same cell. By integrating STAG-seq with base editing in human primary immune cells, both mono-and biallelic effects of coding variants implicated in immune-mediated diseases in primary human macrophages have been uncovered. Furthermore, mechanistic insights into a novel noncoding variant within a complex autoimmunity locus in primary human T cells have been identified herein, and have revealed both cis and trans regulatory effects. This approach advances the ability to precisely dissect the functional impact of genetic variants in their native genomic and cellular contexts.

### Methods and Systems for Analyzing Nucleic Acids

In an aspect, the present disclosure provides methods of analyzing nucleic acids, comprising providing at least one targeting probe to a target nucleic acid, where the targeting probe binds to a target region in the target nucleic acid and includes a sequence capable of sequence-specific hybridization to a barcoding oligonucleotide (optionally also including a UMI). A first sensing oligo can also be provided, whereby the first sensing oligo binds to the at least one targeting probe. A second sensing oligo comprising a sequencing adaptor can also be provided, whereby the second sensing oligo binds to the first sensing oligo via a hybridization region in the second sensing oligo. The second sensing oligo can then be attached to the at least one targeting probe, thereby generating an RNA expression tracking construct of the disclosure. While the RNA expression tracking construct is exposed to a single-cell transcriptome, genotyping probes and bead-attached barcoding oligonucleotides are also introduced to individual discrete volumes (e.g., droplets), under conditions sufficient to allow for RNA expression tracking construct-mediated transcriptome assessment, genotyping probe-mediated amplification and barcoding of both RNA expression tracking constructs and genotyping amplicons to proceed in the individual discrete volumes (e.g., single cell-containing droplets), with the individual discrete volumes then pooled for high-throughput sequencing (e.g., NGS), optionally partitioned between genotyping and transcriptome assessment via size selection (e.g., SPRI-mediated enrichment for genotyping amplicons) and/or affinity selection (e.g., tagging of RNA expression tracking constructs with biotin probes and pulldown via streptavidin binding), with results providing paired target RNA expression levels and genotype information from single cells of an input population of cells.

### Targeting Probes

The methods and systems of the disclosure may include one or more targeting probes. A targeting probe may be an oligonucleotide, e.g., DNA, RNA, or a hybrid thereof, that is capable of binding to a target nucleic acid. The targeting probe may be single stranded or double stranded. In some cases, the targeting probe may include a hairpin structure. In some examples, the targeting probe may be DNA, e.g., single stranded DNA.

The targeting probe may include a region for binding to a target nucleic acid. The region may be substantially complementary to a target region in the target nucleic acid. In some cases, the region is completely complementary to a target region. In some cases, the region may be at least 50%, at least 60%, at least 70%, at least 80%, at least 90%, at least 95%, or at least 99% complementary to a target region. In some cases, the targeting probe may further include a region for binding to a sensing oligo as described herein, and/or a region capable of hybridizing a barcoding oligonucleotide and/or a UMI sequence.

The target region in a target nucleic acid that can be bound by a targeting probe may be from about 5 nucleotides (nt) to about 500 nt, from about 10 nt to about 300 nt, from about 10 nt to about 200 nt, from about 10 nt to about 100 nt, from about 10 nt to about 50 nt, from about 10 nt to about 40 nt, from about 15 nt to about 35 nt, from about 10 nt to about 20 nt, from about 15 nt to about 25 nt, from about 20 nt to about 30 nt, from about 25 nt to about 35 nt, from about 30 nt to about 40 nt, from about 35 nt to about 45 nt, or from about 40 nt to about 50 nt in length. For example, the targeting region may be about 5, about 10, about 15, about 16, about 17, about 18, about 19, about 20, about 21, about 22, about 23, about 24, about 25, about 26, about 27, about 28, about 29, about 30, about 35, about 40, about 50, about 60, about 70, about 80, about 90, or about 100 nt in length. In some examples, the target region is from about 10 to about 200 nt in length. In some examples, the target region is about 25 nt in length.

In some cases, the methods and systems may include one targeting probe, e.g., only one RNA expression targeting probe. In certain cases, the methods and systems may RNA expression a plurality of targeting probes, e.g., 2, 3, 4, 5, 6 or more. In some examples, the methods and systems include 2 targeting probes. In cases where the methods and systems include a plurality of targeting probes, the targeting probes may bind to the same target nucleic acid. For example, the targeting probes may bind to target regions adjacent to each other on a target nucleic acid.

In some embodiments, a targeting probe may include one or more modifications. For example, the targeting probe may include a phosphate. The phosphate may be on the 5′ end of the targeting probe. Alternatively or additionally, the phosphate may be on the 3′ end of the targeting probe. In some cases, the phosphate may facilitate ligation of the targeting probes to another molecule, e.g., a sensing oligo.

### Sensing Oligos

The methods and systems may include one or more sensing oligos. A sensing oligo may be an oligonucleotide, e.g., DNA, RNA, or a hybrid thereof, that binds to one or more targeting probes described herein. The sensing oligo may be single stranded or double stranded. In some cases, the sensing oligo may include a hairpin structure. In some examples, the sensing oligo may be DNA, e.g., single stranded DNA.

The sensing oligo may include one or more binding regions for binding with one or more targeting probes. In some embodiments, a sensing oligo binds to multiple targeting probes. In such cases, the sensing oligo may include multiple binding regions, each binding region binding to a targeting probe.

The binding region of a sensing oligo may be substantially complementary to sequence in a targeting probe. In some cases, the region is completely complementary to a sequence in a targeting probe. In some cases, the binding region may be at least 50%, at least 60%, at least 70%, at least 80%, at least 90%, at least 95%, or at least 99% complementary to a sequence in a targeting probe.

A sensing oligo's binding region that binds to a targeting probe may be from about 5 nt to about 500 nt, from about 10 nt to about 300 nt, from about 10 nt to about 200 nt, from about 10 nt to about 100 nt, from about 10 nt to about 50 nt, from about 10 nt to about 40 nt, from about 15 nt to about 35 nt, from about 10 nt to about 20 nt, from about 15 nt to about 25 nt, from about 20 nt to about 30 nt, from about 25 nt to about 35 nt, from about 30 nt to about 40 nt, from about 35 nt to about 45 nt, or from about 40 nt to about 50 nt in length. For example, the binding region may be about 5, about 10, about 15, about 16, about 17, about 18, about 19, about 20, about 21, about 22, about 23, about 24, about 25, about 26, about 27, about 28, about 29, about 30, about 35, about 40, about 50, about 60, about 70, about 80, about 90, or about 100 nt in length. In some examples, the binding region is from about 10 to about 100 nt in length.

In some cases, the methods and systems include one sensing oligo, e.g., only one sensing oligo. In certain cases, the methods and systems include a plurality of, e.g., 2, 3, 4, 5, 6 or more, sensing oligos. In some examples, the methods and systems may include 2 sensing oligos. In cases where the methods and systems include a plurality of sensing oligos, the sensing oligos may bind to each other.

A sensing oligo may bind to another sensing oligo via a hybridization region. The hybridization region may be 20 nt or less, 18 nt or less, 16 nt or less, 14 nt or less, 12 nt or less, 10 nt or less, 8 nt or less, 6 nt or less, 4 nt or less, 2 nt or less, or 1 nt or less in length. In some examples, a sensing oligo may have a hybridization region that is 10 nt or less in length. In some cases, the hybridization region may be 1, 2, 3, 4, 5, 6, 7, 8, 9, or 10 nt in length. In one example, the hybridization region may be 2 nt in length. In one example, the hybridization region may be 3 nt in length. In some examples, however, a sensing oligo does not have any hybridization region that binds to another sensing oligo.

### Binding of Targeting Probe and Target Nucleic Acid

The methods may include providing one or more targeting probes so that the targeting probe(s) binds to a target nucleic acid. In certain cases, the target nucleic acid may be RNA, such as mRNA, tRNA, rRNA, microRNA, cell-free RNA, non-polyadenylated transcripts, and synthetic RNA. In certain cases, the target nucleic acid may include a hybrid of DNA and RNA. In some examples, the target nucleic acid is RNA. In some embodiments, the target nucleic acid includes mRNA or DNA derived therefrom. In some cases, the target nucleic acid may be DNA, such as genomic DNA, DNA in organelles (e.g., mitochondrial DNA or chloroplast DNA), cell-free DNA, cDNA, synthetic DNA, or any combination thereof.

When binding to the targeting probe(s), the target nucleic acid may be in a nucleus, in the cytoplasm, or elsewhere within a cell, or outside a cell. In some cases, the target nucleic acid may be outside a nucleus when binding to the targeting probe(s). When the target nucleic acid is in a cell or nucleus, the cell or nucleus may be fixed, e.g., using methods described herein and/or as known in the art.

The method may include providing multiple targeting probes so the multiple targeting probes bind to multiple target regions in the target nucleic acid. For example, the methods may include providing a first and a second targeting probe, which bind to a first and a second target region in the target nucleic acid, respectively.

In some cases, the first and the second target regions may be adjacent to each other. For example, the first and the second target regions may have a distance therebetween that is 50 nt or less, 40 nt or less, 30 nt or less, 20 nt or less, 10 nt or less, 9 nt or less, 8 nt or less, 7 nt or less, 6 nt or less, 5 nt or less, 4 nt or less, 3 nt or less, 2 nt or less, or 1 nt or less. In some cases, the first and the second target regions have a distance therebetween that is 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, or 20 nt. In some examples, the first and the second target regions have a distance therebetween of 2 nt.

In certain embodiments, the at least one targeting probe is a first targeting probe, and the method may further include providing a second targeting probe, whereby the first and the second targeting probes bind to first and a second target regions in the target nucleic acid, respectively.

The target region in a target nucleic acid that can be bound by a targeting probe may be from about 5 nucleotides (nt) to about 500 nt, from about 10 nt to about 300 nt, from about 10 nt to about 200 nt, from about 10 nt to about 100 nt, from about 10 nt to about 50 nt, from about 10 nt to about 40 nt, from about 15 nt to about 35 nt, from about 10 nt to about 20 nt, from about 15 nt to about 25 nt, from about 20 nt to about 30 nt, from about 25 nt to about 35 nt, from about 30 nt to about 40 nt, from about 35 nt to about 45 nt, or from about 40 nt to about 50 nt in length. For example, the targeting region may be about 5, about 10, about 15, about 16, about 17, about 18, about 19, about 20, about 21, about 22, about 23, about 24, about 25, about 26, about 27, about 28, about 29, about 30, about 35, about 40, about 50, about 60, about 70, about 80, about 90, or about 100 nt in length. In some examples, the target region is from about 10 to about 200 nt in length. In some examples, the target region is about 25 nt in length.

In some embodiments, the target nucleic acid may be in a cell. In some embodiments, the method may further include fixing or crosslinking the cell, as described elsewhere herein. In some embodiments, the oligos detect nucleic acids inside of cells, and the cells are first fixed and permeabilized prior to applying the oligos.

In some embodiments, the targeting probe may include a 5′ phosphate.

Binding of Sensing Oligo with Targeting Probe and Another Sensing Oligo

The method may include providing one or more sensing oligos so that the sensing oligo(s) binds to one or more targeting probes. In some cases, the method includes providing a sensing oligo so that that it binds to at least one, e.g., 1, 2, 3, 4, 5, 6, or more targeting probes. In some examples, the sensing oligo binds to one targeting probe. In some examples, the sensing oligo binds to two targeting probes.

In some examples, the sensing oligo binds to a first and a second targeting probes. In such cases, the first and the second targeting probes bind to a first and a second binding regions in the first sensing oligo, respectively.

In some cases, the first and the second binding target regions may be adjacent to each other. For example, the first and the second binding target regions may have a distance therebetween that is 50 nt or less, 40 nt or less, 30 nt or less, 20 nt or less, 10 nt or less, 9 nt or less, 8 nt or less, 7 nt or less, 6 nt or less, 5 nt or less, 4 nt or less, 3 nt or less, 2 nt or less, or 1 nt or less. In some cases, first and the second target regions have a distance therebetween that is 1 nt, 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, or 20 nt. In some examples, the first and the second binding regions has a distance therebetween of 2 nt. The second sensing oligo may bind to the first sensing oligo via a hybridization region on the second sensing oligo, which may be less than 10 nt, less than 5 nt or less than 3 nt in length.

The binding of the sensing oligo with the targeting probe(s) may be regulated. In some cases, the binding may be thermodynamically regulated. For example, the binding may be regulated by incubating the reaction at a certain temperature.

In some embodiments, the methods include providing multiple sensing oligos, including a first and a second sensing oligo. The first sensing oligo may bind to the targeting probe(s) as described above. The second sensing oligo may bind to the first sensing oligo via a hybridization region on the second sensing oligo.

In certain embodiments, the first and second targeting probes may bind to first and second binding regions in the first sensing oligo, respectively. In specific embodiments, the first or the second binding region may be from 10 nt to 100 nt in length, as described elsewhere herein.

### Hairpin Structure and Initiator

A sensing oligo may include a hairpin structure, e.g., when not binding to other molecules.

The hairpin structure may open when the sensing oligo binds to another molecule, such as a targeting probe or another sensing oligo.

In some cases, the hairpin structure in the sensing oligo may include one or more secondary structure units, e.g., one or more loops. the secondary structures may prevent the sensing oligo from binding to another molecule. The secondary structures may be metastable under the reaction conditions in the absence of an initiator nucleic acid. In the presence of an initiator, the secondary structures may change such that the sensing oligo may hybridize to another molecule such as a targeting probe and/or another sensing oligo.

The secondary structure in a sensing oligo may open when it binds to an initiator. The initiator may be a nucleic acid molecule that includes a region substantially complementary to a portion of the sensing oligo. In some cases, the initiator may be a targeting probe. In certain cases, the initiator may be a sensing oligo.

In other embodiments, the initiator includes at least a portion of a nucleic acid that is part of a “initiation trigger” such that the initiator is made available when a predetermined physical event occurs. The predetermined event may be the presence of an analyte of interest. In certain embodiments, the predetermined event may be any physical process that exposes the initiator. For example, and without limitation, the initiator may be exposed as a result of a change in temperature, pH, the magnetic field, or conductivity. In each of these embodiments the initiator may be associated with a molecule that is responsive to the physical process. Thus, the initiator and the associated molecule together form the initiation trigger. For example, the initiator may be associated with a molecule that undergoes a conformational change in response to the physical process. In other embodiments, however, the initiation trigger includes a single nucleic acid. The initiator region of the nucleic acid is made available in response to a physical change. For example, the conformation of the initiation trigger may change in response to pH to expose the initiator region. The conformational change may expose the binding site for a sensing oligo in the initiator sequence.

The structure of the trigger may be such that when the analyte of interest is not present (or the other physical event has not occurred), the initiator is not available to hybridize with the sticky end of a monomer. Analyte frees the initiator such that it can interact with a metastable monomer.

In some embodiments, analyte causes a conformational change in the trigger that allows the initiator to interact with the sensing oligo.

Sensing oligos with hairpin structures and initiators may include those described in U.S. Pat. No. 11,414,701, as well as in US20180010166A1 and U.S. Pat. No. 7,632,641.

In some embodiments, when attached, the second sensing oligo and the at least one targeting probe may form a loop structure. In some embodiments, the first or the second sensing oligo may include a hairpin structure when not binding to other molecules. The hairpin structure may open when the first or the second sensing oligo binds to the at least one targeting probe, or when the first and the second sensing oligo bind to each other.

In some embodiments, the first and the second sensing oligos may be included in the same molecule.

In some embodiments, the targeting probe or sensing oligo may include a UMI and/or sequence capable of annealing to a barcoding oligonucleotide, as described elsewhere herein.

### Ligation

In such cases, the methods may further include attaching the second sensing oligo to a targeting probe. The construct resulting from the attachment may be an RNA expression tracking construct as described herein. The attachment may be performed by ligation. In some cases, the method may further include modifying (e.g., adding a phosphate to the 5′ end) the second sensing oligo and/or the targeting probe to facilitate ligation. In some embodiments, the second sensing oligo may include a primer binding site. In some embodiments, the second sensing oligo may be attached to the at least one targeting probe by ligation using a ligase as described herein.

As used herein, the term “ligation” refers to joining two or more nucleic acid molecules. The ligation may be performed using a ligase. A ligase may refer to an enzyme that is capable of ligating nucleic acid. For example, a ligase may be capable of ligating the 3′-end of an acceptor polynucleotide to a the 5′-end of a donor polynucleotide. Examples of ligases include bacteriophage T4 DNA ligase, Escherichia coli (E. coli) DNA ligase, Aquifex aeolicus DNA ligase, Thermus aquaticus(Taq) DNA ligase, 9° N™ DNA ligase, Methanobacterium thermoautotrophicum RNA ligase, Ferroplasma acidiphilum DNA ligase, Human DNA ligase I, Human DNA ligase II, Human DNA ligase III, Human DNA ligase IV, Vaccinia virus DNA ligase, Chlorella virus DNA ligase, Pyrococcus furiosis DNA ligase, Haloferax volcanii DNA ligase, Acidianus ambivalens DNA ligase, Archaeoglobus fulgidus DNA ligase, Aeropyrum pernix DNA ligase, Cenarcheon symbiosum DNA ligase, Haloarcula marismortui DNA ligase, Ferroplasma acidarmanus DNA ligase, Natronomonas pharaonis DNA ligase, Haloquadratum walsbyi DNA ligase, Halobacterium salinarum DNA ligase, Methanosarcina acetivorans DNA ligase, Methanosarcina barkeri DNA ligase, Methanococcoides burtonii DNA ligase, Methanospirillum hungatei DNA ligase, Methanocaldococcus jannaschii DNA ligase, Methanopyrus kandleri DNA ligase, Methanosarcina mazei DNA ligase, Methanococcus maripaludis DNA ligase, Methanosaeta thermophile DNA ligase, Methanosphaera stadtmanae DNA ligase, Methanothermobacter thermautotrophicus DNA ligase, Nanoarchaeum equitans DNA ligase, Pyrococcus abyssi DNA ligase, Pyrobaculum aerophilum DNA ligase, Pyrococcus horikoshii DNA ligase, Picrophilus torridus DNA ligase, Sulfolobus acidocaldarius DNA ligase, Sulfolobus shibatae DNA ligase, Sulfolobus solfataricus DNA ligase, Sulfolobus tokodaii DNA ligase, Thermoplasma acidophilum DNA ligase, Thermococcus fumicolans DNA ligase, Thermococcus kodakarensis DNA ligase, Thermococcus sp. NA1 DNA ligase, Thermoplasma volcanium DNA ligase, Staphylococcus aureus DNA ligase, Thermus scotoductus NAD+-DNA ligase, T4 RNA ligase, Staphylococcus aureus DNA ligase, Methanobacterium thermoautotrophicum DNA ligase, Thermus species AK16D DNA ligase, Haemophilus influenzae DNA ligase, Thermus thermophilus DNA ligase, bacteriophage T7 DNA ligase, Haemophilus influenzae DNA ligase, Mycobacterium tuberculosis DNA ligase, Deinococcus radiodurans RNA ligase, Methanobacterium thermoautotrophicum RNA ligase, Rhodothermus marinus RNA ligase, Trypanosoma brucei RNA ligase, bacteriophage T4 RNA ligase 1, Ampligase, and bacteriophage T4 RNA ligase 2. In some examples, the ligase may be T4 ligase. In some examples, the ligase may be T7 ligase.

### Amplification

In some embodiments, the RNA expression tracking constructs generated herein and/or genomic DNA may be amplified. The amplification may be performed using amplification primers. The primers may bind to primer binding site(s), adaptor(s), and/or barcode(s) on the RNA expression tracking constructs and/or genomic DNA (e.g., using genomic target site-recognizing genotyping probes).

The amplification may be performed using methods described herein. Examples of amplification techniques that can be used include, but are not limited to, PCR, quantitative PCR, quantitative fluorescent PCR (QF-PCR), multiplex fluorescent PCR (MF-PCR), real time PCR, reverse transcription PCR (RT-PCR), single cell PCR, restriction fragment length polymorphism PCR (PCR-RFLP), hot start PCR, nested PCR, in situ polony PCR, in situ rolling circle amplification (RCA), bridge PCR, picotiter PCR, and emulsion PCR. Other suitable amplification methods include the ligase chain reaction (LCR), transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR), and nucleic acid sequence-based amplification (NASBA).

In some embodiments, the systems and methods described herein may include one or more primers configured to bind to at least a portion of the first targeting probe. In some embodiments, the systems and methods described herein may include one or more primers configured to bind to at least a portion of the second sensing oligo.

### Sequencing

The methods herein may further include sequencing one or more of the RNA expression tracking construct(s) (and/or amplicons thereof), the one or more genotyping amplicons (gene-specific amplicons), or a portion thereof. In some embodiments, the RNA expression tracking construct(s) may include at least a portion of the second sensing oligo and at least a portion of the at least one targeting probe (optionally including a UMI and/or a region capable of annealing a barcoding oligonucleotide), as described herein. In certain embodiments, the version of the RNA expression tracking construct that is ultimately sequenced has been barcoded while in an individual discrete volume in the presence of bead-attached barcoding oligonucleotides (thereby allowing for identification of the origin individual discrete volume for the RNA expression tracking construct).

In some cases, the sequencing may be next generation sequencing. The terms “next-generation sequencing” or “high-throughput sequencing” refer to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies or single-molecule fluorescence-based method commercialized by Pacific Biosciences. Any method of sequencing known in the art can be used before and after isolation. In certain embodiments, a sequencing library is generated and sequenced.

At least a part of the RNA expression tracking construct(s) (and/or amplicons thereof), the one or more genotyping amplicons (gene-specific amplicons), or portion(s) thereof generated by the methods herein may be sequenced to produce a plurality of sequence reads. The fragments may be sequenced using any convenient method. For example, the fragments may be sequenced using Illumina's reversible terminator method, Roche's pyrosequencing method (454), Life Technologies' sequencing by ligation (the SOLiD platform) or Life Technologies' Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513:19-39) and Morozova et al (Genomics. 2008 92:255-64), which are incorporated by reference for the general descriptions of the methods and the particular steps of the methods, including all starting products, methods for library preparation, reagents, and final products for each of the steps. As would be apparent, forward and reverse sequencing primer sites that are compatible with a selected next generation sequencing platform can be added to the ends of the fragments during the amplification step. In certain embodiments, the fragments may be amplified using PCR primers that hybridize to the tags that have been added to the fragments, where the primer used for PCR have 5′ tails that are compatible with a particular sequencing platform.

In some cases, the sequencing may be performed at certain “depth.” The terms “depth” or “coverage” as used herein refers to the number of times a nucleotide is read during the sequencing process. In regards to single cell RNA sequencing, “depth” or “coverage” as used herein refers to the number of mapped reads per cell. Depth in regards to genome sequencing may be calculated from the length of the original genome (G), the number of reads (N), and the average read length (L) as N×L/G. For example, a hypothetical genome with 2,000 base pairs reconstructed from 8 reads with an average length of 500 nucleotides will have 2× redundancy.

In some cases, the sequencing herein may be low-pass sequencing. The terms “low-pass sequencing” or “shallow sequencing” as used herein refers to a wide range of depths greater than or equal to 0.1× up to 1×. Shallow sequencing may also refer to about 5000 reads per cell (e.g., 1,000 to 10,000 reads per cell).

In some cases, the sequencing herein may be deep sequencing or ultra-deep sequencing.

The term “deep sequencing” as used herein indicates that the total number of reads is many times larger than the length of the sequence under study. The term “deep” as used herein refers to a wide range of depths greater than 1x up to 100x. Deep sequencing may also refer to 100x coverage as compared to shallow sequencing (e.g., 100,000 to 1,000,000 reads per cell). The term “ultra-deep” as used herein refers to higher coverage (>100-fold), which allows for detection of sequence variants in mixed populations.

In some embodiments, the method may further include analyzing the target nucleic acid based, at least in part, on the sequence read of the construct that is sequenced (e.g., the RNA expression tracking construct and/or the genotyping amplicon), as described herein. In some embodiments, analyzing the target nucleic acid may include quantifying the target nucleic acid. In some embodiments, the method involves amplifying the construct that is sequenced, thereby generating a sequencing library comprising the amplified construct.

### Sequencing Construct

The methods and systems of the disclosure may further include one or more sequencing constructs. A sequencing construct is a polynucleotide that can be sequenced. In some embodiments, a sequencing construct may be an RNA expression tracking construct (e.g., an RNA expression tracking construct that also includes a barcode sequence added using bead-attached barcoding oligonucleotides) that includes at least a portion of a sensing oligo and at least a portion of a targeting probe. The sequencing construct may include one or more sequencing adaptors, one or more barcodes, and one or more target region of interest. In additional embodiments, a sequencing construct can include a gene-specific amplicon (e.g., a genotyping amplicon that further includes a barcode sequence added using bead-attached barcoding oligonucleotides).

In some cases, the methods and systems may include a sequencing library that includes a plurality of sequencing constructs or amplicons thereof.

### Unique Molecular Identifiers (UMIs)

Certain aspects of the disclosure feature unique molecular identifier (UMI) sequences. Exemplary synthesis of a UMI involves: Following the completion of “split-and-pool” synthesis cycles for generation of barcodes, all microparticles are together subjected to eight rounds of degenerate synthesis with all four DNA bases available during each cycle, such that each individual primer receives one of 48 (65,536) possible sequences (UMIs). A UMI is thereby provided that allows distinguishing between, e.g., individual bead-attached oligonucleotides upon the same bead which otherwise share a common barcode (being that such oligonucleotides are attached to the same bead and therefore receive the same spatial barcode). It is specifically contemplated that any probe or oligonucleotide of the disclosure (e.g., targeting probe, sensing oligo, and/or readout oligo) may comprise a UMI, a barcode sequence, and/or other functional sequence as described herein or as otherwise known in the art.

### Adaptors

The targeting probe herein may include one or more adaptors. Alternatively or additionally, the sensing oligo may include one or more adaptors. In some examples, a targeting probe may include an adaptor at its 5′ end and a sensing oligo may include an adaptor at its 5′ end.

An adaptor may be an oligonucleotide that can be attached to one or more nucleic acids. The adaptor may include a plurality of oligonucleotides. The adaptor may include DNA, RNA, or a hybrid thereof. The adaptor may be single stranded, double-stranded, or a mixture thereof. The adaptor may include a unique molecular identifier (UMI), sample index, primer sequence, linker sequence, or a combination thereof. The UMI may be adjacent to the sample index. The UMI may be adjacent to the primer sequence. The sample index may be adjacent to the primer sequence. A linker sequence may connect a UMI to a sample index. A linker sequence may connect the UMI to the primer sequence. A linker sequence may connect the sample index to the primer sequence.

An adaptor may be a molecule configured to accept or receive a barcode. In some examples, the adaptor may include an overhang, and the barcoding oligonucleotide may include a sequence capable of hybridizing to the overhang. For example, an adaptor may include a single-stranded nucleic acid sequence (for example, an overhang) capable of hybridizing to a barcoding oligonucleotide and/or a given barcode, for example, via a sequence complementary to a portion or the entirety of the barcoding oligonucleotide. In certain embodiments, this portion of the barcoding oligonucleotide is a standard sequence held constant between individual barcoding oligonucleotides. The hybridization couples the adaptor to the barcoding oligonucleotide. In some embodiments, the adaptor may be associated with (for example, attached to) a target molecule. As such, the adaptor may serve as the means through which a barcode is attached to a target molecule.

An adaptor may be attached to a target molecule according to methods known in the art. For example, a barcode receiving adaptor may be attached to a polypeptide target molecule at a cysteine residue (for example, a C-terminal cysteine residue). An adaptor may be used to identify a particular condition related to one or more target molecules, such as a cell of origin or a discrete volume of origin. For example, a target molecule can be an mRNA expressed by a cell, which receives a cell-specific adapter. The barcode receiving adaptor can be conjugated to one or more barcodes as the cell is exposed to one or more conditions, such that the original cell of origin for the target molecule, optionally as well as each condition to which the cell was exposed, can be subsequently determined by identifying the sequence of the barcode receiving adaptor/barcode concatemer.

### Sequencing Adaptor

In some embodiments, the adaptor may be a sequencing adaptor. The term “sequencing adaptor,” as used herein, generally refers to a molecule (e.g., polynucleotide) that is adapted to permit a sequencing instrument to sequence a target polynucleotide, such as by interacting with the target polynucleotide to enable sequencing. The sequencing adaptor may permit the target polynucleotide (be it a barcoded RNA expression tracking construct, a barcoded gene-specific amplicon, etc.) to be sequenced by the sequencing instrument. In an example, the sequencing adaptor may include a nucleotide sequence that hybridizes or binds to a capture polynucleotide attached to a solid support of a sequencing system, such as a flow cell. In another example, the sequencing adaptor includes a nucleotide sequence that hybridizes or binds to a polynucleotide to generate a hairpin loop, which permits the target polynucleotide to be sequenced by a sequencing system. The sequencing adaptor can include a sequencer motif, which can be a nucleotide sequence that is complementary to a flow cell sequence of another molecule (e.g., polynucleotide) and usable by the sequencing system to sequence the target polynucleotide. The sequencer motif can also include a primer sequence for use in sequencing, such as sequencing by synthesis. The sequencer motif can include the sequence(s) needed to couple a library adaptor to a sequencing system and sequence the target polynucleotide. In some cases, the targeting probe and/or genotyping probe may include a sequencing adaptor, e.g., at its 5′ end. In certain cases, the sensing oligo may include a sequencing adaptor, e.g., at its 5′ end.

In some cases, the sequencing adaptor may be from about 5 nt to about 50 nt, from about 5 nt to about 40 nt, from about 5 nt to about 30 nt, from about 5 nt to about 15 nt, from about 10 nt to about 20 nt, from about 15 nt to about 25 nt, or from about 20 nt to about 30 nt in length. In some examples, the sequencing adaptor may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nt in length.

In certain embodiments, the at least one targeting probe, one or more sensing probes and/or one or more gene-specific probe (genotyping probe) includes or is linked to sequencing adapters (for example, universal primer recognition sequences) such that the probe and sequencing adapter elements are both coupled to the target molecule. In particular examples, the sequence of the probe is amplified, for example using PCR. In some embodiments, the targeting probe, sensing probe and/or gene-specific probe may include a primer binding site. As such, the targeting probe, sensing probe and/or gene-specific probe may include a molecular identifier sequence.

In some embodiments, the adaptor in the second sensing probe is at a 3′ side of the hybridization region.

### Primer Binding Sites

The targeting probe, sensing probe and/or gene-specific probe may include one or more primer binding sites. The primer binding site may be a sequence capable of hybridizing with one or more primers, e.g., sequencing primers, amplification primers, etc. In some examples, a primer binding site may be in an adaptor described herein.

### Primers

The system may further include one or more primers. The primers may be used for amplification, sequencing, nucleic acid detecting, nucleic acid capturing, or a combination thereof. The primers may bind to the primer binding sites described herein. In some cases, a primer may be configured to bind to at least a portion of a targeting probe, sensing probe and/or gene-specific probe. In some cases, a primer may be configured to bind to at least a portion of the second sensing oligo.

### Barcode

The targeting probe, sensing probe and/or gene-specific probe may include one or more barcode(s) as described herein. In some cases, the barcode may include a unique molecular identifier (UMI) as defined herein. In one example, the targeting probe includes a UMI.

The term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Barcoding may be performed based on any of the compositions or methods disclosed in patent publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell. A nucleic acid barcode or can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single-or double-stranded form. Target molecule and/or target nucleic acids can be labeled with multiple nucleic acid barcodes in combinatorial fashion, such as a nucleic acid barcode concatemer.

Typically, a nucleic acid barcode is used to identify a target molecule and/or target nucleic acid as being from a particular discrete volume, having a particular physical property (for example, affinity, length, sequence, etc.), or having been subject to certain treatment conditions. A target molecule and/or target nucleic acid can be associated with multiple nucleic acid barcodes to provide information about all of these features (and more).

In some embodiments, a sequence of the disclosure may include a unique molecular identifier (UMI). The term “unique molecular identifiers” as used herein refers to a sequencing linker or a subtype of nucleic acid barcode used in a method that uses molecular tags to detect and quantify unique amplified products. A UMI is used to distinguish effects through a single clone from multiple clones. The term “clone” as used herein may refer to a single mRNA or target nucleic acid to be sequenced. The UMI may also be used to determine the number of transcripts that gave rise to an amplified product, or in the case of target barcodes as described herein, the number of binding events. In preferred embodiments, the amplification is by PCR, multiple displacement amplification (MDA), or isothermal amplification.

A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form.

### Methods for Generating Single-Cell Molecular Analysis

In certain example embodiments, the methods for monitoring expression and/or genotyping single cells rely, at least in part, on ligation dependent probes. A ligation dependent probe is a probe that includes a target binding region configured to bind a target polynucleotide and a primer binding site region. Ligation dependent probes may be used in a set of two or more. Ligation dependent probes may include a set of individual ligation dependent probes, with each individual ligation dependent probe configured to hybridize to a specific target nucleic acid sequence on a target polynucleotide. Target sequences on the target polynucleotide are selected to be close enough in distance on the target polynucleotide such that ligation dependent probes hybridized to said target nucleic acid sequences may be subsequently ligated together. Accordingly, in certain embodiments, ligation dependent probe pairs may bind within 1 nucleotide of one another. In some embodiments, the ligation dependent probe pairs may bind within 2 to 500 nucleotides of one another, the gap between which is filled through polymerase extension, or another polynucleotide filler, prior to ligation. Alternatively, a ligation dependent probe may be a single molecule comprising two or more target binding regions connected by linker sequences. The target binding regions include a nucleic acid sequence selected to hybridize to a target region on a target polynucleotide. Linker sequences are selected such that the molecule may adapt a conformation that allows the individual target binding regions to hybridize to adjacent regions on the target polynucleotide. Target sequences on the target polynucleotide are selected to be close enough in distance on the target polynucleotide such that ligation dependent probes hybridized to said target nucleic acid sequences may be subsequently ligated together. Accordingly, in certain embodiments, ligation dependent probe pairs may bind within 1, 2, 3, 4, or 5 nucleotides of one another. In certain example embodiments, the ligation dependent probes comprising two or more target binding regions may be based on molecule inversion probes (MIP), or “padlock probes.” See e.g. Niedzicka et al. Sci Rep. 2016; 6:24501.

In the case of padlock probes and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probes, probe concatemers, and the like, with an exonuclease, such as exonuclease I.

Ligation dependent probes may be RNA, DNA, or a combination thereof. Ligation dependent probes may vary in length from 10 to 200 nucleotides. To allow for amplification, the ligation dependent probes may further include a primer binding site. The same or different primer binding site may be found on each ligation dependent probe. In certain embodiments, a set of ligation dependent probes is provided, each ligation dependent probe including a target binding region to a different target nucleic acid sequence on the same or different target polynucleotide, but the same primer binding set on each ligation dependent probe.

In one embodiment, the ligation dependent probes are designed to bind one or more target RNA molecule in a cell. The ligation dependent probes may be configured to bind to select RNA fragments or RNA exons for the purpose of quantifying the amount of the selected RNA fragment or exon in a sample, or configured to hybridize to a specific RNA sequence variant to detect and identify the presence of said variant in a sample.

Ligation dependent probes are delivered to a sample containing the target molecules of interest. The method of delivery will depend on the sample type. Samples sources may include biological samples of a subject, or environmental samples. These samples may be solids or liquids. The biological samples may include, but are not limited to, animal tissues such as those obtained by biopsy or post mortem, including saliva, blood, semen, plasma, sera, stool, urine, sputum, mucous, lymph, synovial fluid, spinal fluid, cerebrospinal fluid, a swab from skin or a mucosal membrane, or combination thereof. Other biological samples may include plant tissues such as leaves, roots, stems, fruit, and seeds, or sap or other liquids obtained when plant tissues are cut or plant cells are lysed or crushed. Environmental samples may include surfaces or fluids. In an example embodiment, the environmental sample is taken from a solid surface, such as a surface used in the preparation of food or other sensitive compositions and materials.

In some embodiments, the ligation dependent probes may be split-ligation probes, each probe further comprising a unique molecular identifier (UMI).

In certain embodiments, a UMI with a random sequence of between 4 and 20 base pairs is added to a template, which is amplified and sequenced. In some embodiments, the UMI is added to the 5′ end of the template. Sequencing allows for high resolution reads, enabling accurate detection of true variants. As used herein, a “true variant” will be present in every amplified product originating from the original clone as identified by aligning all products with a UMI. Each clone amplified will have a different random UMI that will indicate that the amplified product originated from that clone. Background caused by the fidelity of the amplification process can be eliminated because true variants will be present in all amplified products and background representing random error will only be present in single amplification products (See e.g., Islam S. et al., 2014. Nature Methods No:11, 163-166). Not being bound by a theory, the UMI's are designed such that assignment to the original can take place despite up to 4-7 errors during amplification or sequencing. Not being bound by a theory, an UMI may be used to discriminate between true barcode sequences.

UMIs can be used, for example, to normalize samples for variable amplification efficiency. For example, in various embodiments, featuring a solid or semisolid support (for example a hydrogel bead), to which nucleic acid barcodes (for example a plurality of barcodes sharing the same sequence) are attached, each of the barcodes may be further coupled to a unique molecular identifier, such that every barcode on the particular solid or semisolid support receives a distinct unique molecule identifier. A unique molecular identifier can then be, for example, transferred to a target molecule with the associated barcode, such that the target molecule receives not only a nucleic acid barcode, but also an identifier unique among the identifiers originating from that solid or semisolid support.

Each member of a given population of UMIs, on the other hand, is typically associated with (for example, covalently bound to or a component of the same molecule as) individual members of a particular set of identical, specific (for example, discrete volume-, physical property-, or treatment condition-specific) nucleic acid barcodes. Thus, for example, each member of a set of origin-specific nucleic acid barcodes, or other nucleic acid identifier or connector oligonucleotide, having identical or matched barcode sequences, may be associated with (for example, covalently bound to or a component of the same molecule as) a distinct or different UMI.

### Lysis Buffers and Proteases

The lysis buffer employed herein, which includes proteases, was obtained from the Mission Bio Tapestri Single-Cell DNA Sequencing kit.

### Droplet-Based Methods

In some embodiments, the disclosure provides methods for generating single-cell molecular analysis comprising: a) delivering one or more RNA expression tracking probes, sensing probes, and/or gene-specific probes to a cell population, wherein each probe includes a target binding region configured to bind one or more target RNAs and/or DNAs and optionally a primer binding site region; b) linking bound probes; c) isolating single cells from the cell population in separate individual discrete volumes, the individual discrete volumes further including a primer pair and amplification reagents, where the primer pair binds to the primer binding sites of the proximity dependent probes, and where at least one primer includes a barcode sequence that uniquely identifies the individual discrete volume; d) amplifying the ligated probes using the primer pair, where the barcode is incorporated into each resulting amplicon; and e) quantifying target RNAs in each individual cell based at least in part on sequencing the resulting amplicons.

An “individual discrete volume” is a discrete volume or discrete space, such as a container, receptacle, or other defined volume or space that can be defined by properties that prevent and/or inhibit migration of nucleic acids and reagents necessary to carry out the methods disclosed herein, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the used of non-walled, or semipermeable is that some reagents, such as buffers, chemical activators, or other agents may be passed out through the discrete volume, while other material, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain example embodiments, the individual discrete volumes are the wells of a microplate. In certain example embodiments, the microplate is a 96 well, a 384 well, or a 1536 well microplate.

Each individual discrete volume further includes a primer pair and amplification reagents, optionally further including RNA expression tracking probes, sensing probes, gene-specific probes, bead-attached barcoding oligonucleotides, enzymes (polymerases, ligases, etc.), lysis buffer and reagents, proteases, etc., in the individual discrete volume implementations/steps described for the methods set forth herein. Primer pairs can include a nucleic acid sequence designed to hybridize to the primer binding sites of certain probes, e.g., RNA expression tracking probes, sensing probes, other ligation dependent probes, and/or genotyping probes. Where the same primer binding site is found on each probe to be amplified, then each individual discrete volume may be loaded with the same primer pair. In specific embodiments, each individual discrete volume may further include primer pairs for genotyping one or more genomic loci on target nucleic acids. In some embodiments, genotyping multiple genomic loci can be achieved by use of multiple primer pairs. Thus, each primer pair may include a barcode sequence that uniquely identifies each individual discrete volume (in certain embodiments, with barcoding of expression tracking constructs and/or genotyping amplicons achieved via use of individual beads having bead-attached barcoding oligonucleotides and performance of an annealing and polymerase extension event within each individual discrete volume that produces sequence information). In alternative embodiments, multiple genomic loci can be genotyped by use of bridged amplification strategies, by creating amplified product from target nucleic acids with primers containing one variable portion of sequence specific to the target nucleic acid and another constant portion, followed by subsequent amplification by a second set of primers that recognize the constant portions of the first set of primers.

As described elsewhere herein, the term “barcode” as used herein refers to a short sequence of nucleotides (for example, DNA or RNA) that is used as an identifier for an associated molecule, such as a target molecule and/or target nucleic acid, or as an identifier of the source of an associated molecule, such as a cell-of-origin.

In specific embodiments, at least one primer includes a barcode sequence that uniquely identifies the individual discrete volume. Optionally, the barcode sequence is initially harbored within a bead-attached barcoding oligonucleotide, e.g., where individual beads having bead-attached barcoding oligonucleotides are encapsulated into individual discrete volumes at an appropriate barcoding step.

Various amplification strategies may be used, such as PCR, multiple displacement amplification (MDA), rolling circle amplification (RCA), ligase chain reaction (LCR), loop-mediated isothermal amplification, helicase-dependent amplification, recombinase polymerase amplification, nucleic acid sequence-based amplification, or ramification amplification method (RAM).

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present disclosure.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present disclosure and as described herein.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification.

In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reaction conditions for the particular application or materials.

One of skill in the art will understand and be able to optimize reaction conditions to obtain sufficient amplification.

In specific embodiments, the probes and/or probe-derived constructs of the disclosure (e.g., RNA expression tracking constructs, genotyping amplicons) may be amplified using one or more primer pairs, optionally where a barcoding oligonucleotide is employed as a primer. In specific embodiments, the barcode may be incorporated into each resulting amplicon.

PCR may be performed in the individual discrete volumes, generating cell-barcoded amplicons as previously described, and the resulting amplicons may be sequenced.

To identify and/or quantify the target polynucleotides present in the sample, single cell sequencing of the resulting amplification products (“amplicons”) may be used. In some examples, an amplicon is a nucleic acid from a cell, or acellular system, such as mRNA or DNA that has been amplified. The amplicons will incorporate the primer barcode, and for certain constructs (e.g., a RNA expression tracking construct as disclosed herein) a UMI, each of which can allow for the identification of individual molecular species, the individual discrete volume of origin i.e. cell, and a relative assessment of quantity of each molecule. In certain embodiments, the disclosure involves high-throughput single-cell RNA-seq and/or targeted nucleic acid profiling (for example, sequencing, quantitative reverse transcription polymerase chain reaction, and the like) where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncommsl4049; International patent publication number WO 2014210353 A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; and Rosenberg et al., 2017, “Scaling single cell transcriptomics through split pool barcoding” bioRxiv preprint first posted online Feb. 2, 2017, doi: dx.doi.org/10.1101/105163, all the contents and disclosure of each of which are herein incorporated by reference in their entirety.

The method may further include quantifying target RNA and/or genotyping target DNA loci based in part on sequencing the barcode of each amplicon. In one exemplary embodiment, RNA is detected and quantified by sequencing RNA expression tracking constructs (e.g., ligation dependent probes), and DNA is simultaneously detected by sequencing of genomic DNA PCR amplicons. Other exemplary sequencing techniques that may be used are described above.

In some embodiments, the cells or population of cells may be obtained from a biological sample. The biological sample may be obtained from a subject suffering from a disease. The biological sample may be a tumor sample. The tumor may be any tumor. This may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The tumor may also include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, astrocytoma, medulloblastoma, craniopharyngioma, ependymoma, pinealoma, hemangioblastoma, acoustic neuroma, oligodendroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

The method may further include barcoding target nucleic acids using unique nucleic acid identifiers, for example origin-specific barcodes and the like. The nucleic acid identifiers, nucleic acid barcodes, can include a short sequence of nucleotides that can be used as an identifier for an associated molecule, location, or condition. In certain embodiments, the nucleic acid identifier further includes one or more unique molecular identifiers and/or barcode receiving adapters. A nucleic acid identifier can have a length of about, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 base pairs (bp) or nucleotides (nt). In certain embodiments, a nucleic acid identifier can be constructed in combinatorial fashion by combining randomly selected indices (for example, about 1, 2, 3, 4, 5, 6, 7, 8, 9, or 10 indexes). Each such index is a short sequence of nucleotides (for example, DNA, RNA, or a combination thereof) having a distinct sequence. An index can have a length of about, for example, 4, 5, 6, 7, 8, 9, 10, 11, 12, 13, 14, 15, 16, 17, 18, 19, 20, 21, 22, 23, 24, or 25 bp or nt. Nucleic acid identifiers can be generated, for example, by split-pool synthesis methods, such as those described, for example, in International Patent Publication Nos. WO 2014/047556 and WO 2014/143158, each of which is incorporated by reference herein in its entirety, or by split-pool ligation methods as described in Quinodoz et al. (Biorxiv “Higher-order inter-chromosomal hubs shape 3-dimensional genome organization in the nucleus” (2017)).

In certain example embodiments, the method further includes introducing amplification reagents to the individual discrete volume (e.g., the droplet). Labeled target molecules and/or target nucleic acids associated origin-specific nucleic acid barcodes (optionally in combination with other nucleic acid barcodes and/or UMIs as described herein) can be amplified by methods known in the art, such as polymerase chain reaction (PCR). For example, the nucleic acid barcode can contain universal primer recognition sequences (or universal primer binding sequences (UBS)) that can be bound by a PCR primer for PCR amplification and subsequent high-throughput sequencing. In certain embodiments, the nucleic acid barcode includes or is linked to sequencing adapters (for example, universal primer recognition sequences) such that the barcode and sequencing adapter elements are both coupled to the target molecule. In particular examples, the sequence of the origin specific barcode is amplified, for example using PCR. In some embodiments, an origin-specific barcode further includes a sequencing adaptor. In some embodiments, an origin-specific barcode further includes universal priming sites (UPS). A nucleic acid barcode (or a concatemer thereof), a target nucleic acid molecule (for example, a DNA or RNA molecule), a nucleic acid encoding a target peptide or polypeptide, and/or a nucleic acid encoding a specific binding agent may be optionally sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode.

Exemplary next generation sequencing technologies include, for example, Illumina sequencing, Ion Torrent sequencing, 454 sequencing, SOLiD sequencing, and nanopore sequencing amongst others. In some embodiments, the sequence of labeled target molecules is determined by non-sequencing based methods. For example, variable length probes or primers can be used to distinguish barcodes (for example, origin-specific barcodes) labeling distinct target molecules by, for example, the length of the barcodes, the length of target nucleic acids, or the length of nucleic acids encoding target polypeptides. In other instances, barcodes can include sequences identifying, for example, the type of molecule for a particular target molecule (for example, polypeptide, nucleic acid, small molecule, or lipid). For example, in a pool of labeled target molecules containing multiple types of target molecules, polypeptide target molecules can receive one identifying sequence, while target nucleic acid molecules can receive a different identifying sequence. Such identifying sequences can be used to selectively amplify barcodes labeling particular types of target molecules, for example, by using PCR primers specific to identifying sequences specific to particular types of target molecules. For example, barcodes labeling polypeptide target molecules can be selectively amplified from a pool, thereby retrieving only the barcodes from the polypeptide subset of the target molecule pool.

In some embodiments, the oligonucleotides are introduced into the droplets by initially attaching the oligonucleotides to a particle (e.g., a bead, a polymeric particle, etc.), then optionally subsequently releasing the oligonucleotides from the particle after the particle has been incorporated into a droplet. See, e.g., U.S. Pat. Appl. Ser. No. 62/072,944, filed Oct. 30, 2014 or PCT Appl. Ser. No. PCT/US2015/026443, filed on Apr. 17, 2015, entitled “Systems and Methods for Barcoding Nucleic Acids,” each incorporated herein by reference. The oligonucleotides are conjugated to the solid support (bead) in a releasable fashion, for instance by a photocleavable linker, an enzymatically cleavable linker, or a chemically releasable linker. For example, in certain embodiments, the oligonucleotides may also contain a cleavable sequence or linker, or otherwise be releasable from the particles. In certain embodiments, the oligonucleotide may contain one or more cleavable linkers, e.g., that can be cleaved upon application of a suitable stimulus. For example, the cleavable sequence may be a photocleavable linker that can be cleaved by applying light or a cleavable linker that can be cleaved by applying a suitable chemical or enzyme.

In certain other example embodiments, a recombinase polymerase amplification (RPA) reaction may be used to amplify the target nucleic acids. RPA reactions employ recombinases which are capable of pairing sequence-specific primers with a homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter.

After, or during, the RPA reaction, a RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

In specific embodiments, the methods described herein may be used for whole transcriptome RNA sequencing.

In specific embodiments, the RNA expression tracking probes and/or sensing probes may be delivered to individual fixed cells prior to encapsulating the individual fixed cells in the droplet.

In specific embodiments, the one or more RNA expression tracking probes and/or sensing probes may target one or more polymorphic sites in target RNAs to provide an allele-specific RNA readout.

### Targeted Genotyping by PCR

In some embodiments, each individual discrete volume (e.g., droplet) further includes genotyping primer pairs for amplifying one or more genomic loci, at least one primer pair (e.g., after a barcoding process is performed using, e.g., bead-attached barcoding oligonucleotides and polymerase(s) as described herein) including a barcode sequence uniquely identifying each individual discrete volume. In some embodiments, the method may further include amplifying the one or more genomic loci and genotyping each individual cell by sequencing the resulting amplicons.

In specific embodiments, the one or more primer pairs amplify one or more genomic loci of interest to generate DNA amplicons. The amplifying step may include determining a genotype of each individual cell by sequencing the resulting DNA amplicons.

In specific embodiments, a second PCR amplification is performed to add sequencing adapters to the DNA amplicons.

In some embodiments, sequencing methods include Whole Genome Sequencing (WGS).

This process includes determining the sequence of the entire genome of an organism, for example, humans, dogs, mice, viruses or bacteria. It is not necessary that the entire genome actually be sequenced. The WGS methods of the disclosure are those sequencing methods that when applied to a sample of genomic DNA are capable of obtaining the sequence of the entire genome. Whole genome sequencing can be performed using any Next Generation Sequencing technology as described herein.

In certain embodiments, the disclosure encompasses use of single nucleus RNA sequencing. For example, single nuclei can be segregated into discrete volumes. In certain embodiments, single nuclei can be labeled with one or more ligation dependent probes. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the disclosure involves plate based single cell RNA sequencing (see, e.g., Picelli, S. et al., 2014, “Full-length RNA-seq from single cells using Smart-seq2” Nature protocols 9, 171-181, doi: 10.1038/nprot.2014.006).

### Targeted RNA Quantification Via Proximity Probes

In some embodiments, DNA amplicons (e.g., RNA expression tracking constructs) may be derived from proximity dependent probes that are hybridized to RNA and then linked through ligation.

In some embodiments, the proximity dependent probes may be HyPR probes, padlock probes, or split-ligation probes, as described elsewhere herein and/or previously in the art.

In some embodiments, each probe that contributes to RNA expression tracking DNA amplicons that are ultimately sequenced optionally further includes a unique molecular identifier (UMI), as described elsewhere herein.

In some embodiments, one or more proximity dependent probes target at least 1, at least 5, at least 10, at least 20, at least 30, at least 40, at least 50, at least 60, at least 70, at least 80, at least 90, at least 100, at least 200, at least 300, at least 500, at least 1000, or at least 10,000 target RNAs, as described elsewhere herein.

In some embodiments, multiple proximity dependent probes bind to the same target RNA.

In some embodiments, 2 to 100 proximity dependent probes are used per target RNA.

In some embodiments, different numbers of proximity dependent probes are used per target RNA, in order to balance the signal coming from RNAs with different detection efficiencies or abundancies.

In some embodiments, the one or more proximity dependent probes target one or more polymorphic sites in target RNAs to provide an allele-specific RNA readout. In some embodiments, the proximity dependent probes may include probes with a different 5′ nucleotide directly overlapping the polymorphic site such that only a matching probe will be successfully linked.

In some embodiments, the one or more target RNAs may include one or more lncRNAs.

### Perturbation Screening, Drug Screening

In certain embodiments, the population of cells may have been previously exposed to a perturbation. Such perturbation may be a chemical, physical or genetic perturbation, or a combination thereof.

In certain embodiments, the gene signatures described herein are screened by perturbation of target genes within said signatures. Methods and tools for genome-scale screening of perturbations in single cells using CRISPR-Cas9 have been described previously (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; and International publication serial number WO/2017/075294). In certain embodiments, signature genes may be perturbed and the perturbation may be identified and assigned to the gene expression readouts of single cells. In some embodiments, signature genes may be perturbed in single cells and gene expression analyzed. Not being bound by a theory, networks of genes that are disrupted due to perturbation of a signature gene may be determined. Understanding the network of genes effected by a perturbation may allow for a gene to be linked to a specific pathway that may be targeted to modulate the signature and treat a cancer. Thus, in certain embodiments, STAG-seq can be used to discover novel drug targets to allow treatment of specific cancer patients having paired genotype and single cell expression profiles indicative of such drug targeting.

In some featured embodiments, perturbations include insertion of targeted genetic variants and/or disruptions, into a population of cells, with paired genotyping and expression phenotyping occurring at the single cell level to identify genetic variants and/or disruptions that have functional impact upon single cells harboring such genetic variants and/or disruptions (as compared to an appropriate control—e.g., cells of the same population that did not integrate such a genetic perturbation, e.g., as identified by targeted genotyping of specific loci in single cells). In certain embodiments of STAG-seq, genomic perturbations may include variant insertions and/or disruptions made using a CRISPR/Cas system for genome editing (e.g., a CRISPR/Cas9 system, together with specific guide RNA(s)).

In certain embodiments, a cell barcode is added to the RNA in single cells, such that the RNA may be assigned to a single cell. In certain embodiments, a Unique Molecular Identifier (UMI) is added to each individual transcript-targeting probe set. Not being bound by a theory, the UMI allows for determining the capture rate of measured signals, or optionally the binding events or the number of transcripts captured.

In certain embodiments, perturbations of cell populations disclosed herein can involve use of a CRISPR-Cas9 system and droplet single-cell sequencing analysis. In certain embodiments, a CRISPR system is used to create an INDEL at a target gene. In other embodiments, epigenetic screening is performed by applying CRISPRa/i/x technology (see, e.g., Konermann et al. “Genome-scale transcriptional activation by an engineered CRISPR-Cas9 complex” Nature. 2014 Dec. 10. doi: 10.1038/naturel4136; Qi, L. S., et al. (2013). “Repurposing CRISPR as an RNA-guided platform for sequence-specific control of gene expression”. Cell. 152 (5): 1173-83; Gilbert, L. A., et al., (2013). “CRISPR-mediated modular RNA-guided regulation of transcription in eukaryotes”. Cell. 154 (2): 442-51; Komor et al., 2016, Programmable editing of a target base in genomic DNA without double-stranded DNA cleavage, Nature 533, 420-424; Nishida et al., 2016, Targeted nucleotide editing using hybrid prokaryotic and vertebrate adaptive immune systems, Science 353(6305); Yang et al., 2016, Engineering and optimizing deaminase fusions for genome editing, Nat Commun. 7:13330; Hess et al., 2016, Directed evolution using dCas9-targeted somatic hypermutation in mammalian cells, Nature Methods 13, 1036-1042; and Ma et al., 2016, Targeted AID-mediated mutagenesis (TAM) enables efficient genomic diversification in mammalian cells, Nature Methods 13, 1029-1035). Numerous genetic variants associated with disease phenotypes are found to be in a non-coding region of the genome, and frequently coincide with transcription factor (TF) binding sites and non-coding RNA genes. Not being bound by a theory, CRISPRa/i/x approaches may be used to achieve a more thorough and precise understanding of the implication of epigenetic regulation. In one embodiment, a CRISPR system may be used to activate gene transcription. A nuclease-dead RNA-guided DNA binding domain, dCas9, tethered to transcriptional repressor domains that promote epigenetic silencing (e.g., KRAB) may be used for “CRISPRi” that represses transcription. To use dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, other CRISPR-based perturbations are readily compatible with STAG-seq, including alternative editors such as CRISPR/Cpf1. In certain embodiments, CRISPR-based perturbations target RNA molecules using Casl3. In certain embodiments, Cpf1 can be used as the CRISPR enzyme for introducing perturbations. Not being bound by a theory, Cpf1 does not require Tracr RNA and is a smaller enzyme, thus allowing higher combinatorial perturbations to be tested.

In one embodiment, CRISPR/Cas9 may be used to perturb protein-coding genes or non-protein-coding DNA. CRISPR/Cas9 may be used to knockout protein-coding genes by frameshifts, point mutations, inserts, or deletions. An extensive toolbox may be used for efficient and specific CRISPR/Cas9 mediated knockout as described herein, including a double-nicking CRISPR to efficiently modify both alleles of a target gene or multiple target loci and a smaller Cas9 protein for delivery on smaller vectors (Ran, F. A., et al., In vivo genome editing using Staphylococcus aureus Cas9. Nature. 520, 186-191 (2015)). A genome-wide sgRNA mouse library (^{˜}10 sgRNAs/gene) may also be used in a mouse that expresses a Cas9 protein (see, e.g., WO2014204727A1).

In one embodiment, perturbation is by deletion of regulatory elements. Non-coding elements may be targeted by using pairs of guide RNAs to delete regions of a defined size, and by tiling deletions covering sets of regions in pools.

In some embodiments, perturbation of genes may be by CRISPR, RNAi, zinc finger nuclease, transcription activator-like effector nuclease, or meganuclease genetic perturbation screen.

In some embodiments, the genetic perturbation may include gene knock-outs, gene knock-ins, transpositions, inversions, and/or one or more nucleotide insertions, deletions, or substitutions. In some embodiments, genetic perturbation may be confirmed by genotyping the target DNA loci according to any of the methods described herein.

In specific embodiments, certain probes of the disclosure may be configured to detect one or more gene expression products in one or more cell pathways. Cell pathways may include, but are not necessarily limited to, cell development pathways, cancer signaling pathways, and immune response signaling pathways.

In some embodiments, perturbed cells can be exposed to one or more physical perturbations, genetic perturbations, chemical perturbations, or a combination thereof. The one or more physical perturbations may include exposure to different temperatures, pressures, flow rates, pHs, growth media compositions, or gas concentrations. The one or more genetic perturbations may include gene knock-outs, gene knock-ins, transpositions, inversions, and/or one or more nucleotide insertions, deletions, or substitutions. The one or more chemical perturbations may include exposure to one or more therapeutic agents or a concentration range of therapeutic agents.

In some embodiments, two or more perturbations may be done sequentially and one or more rounds of combinatorial indexing may be done between each round of perturbation.

In some embodiments, one or more expression tracking probes are configured to detect one or more gene expression products in one or more cell pathways.

### Droplet Formation Processes and Kits

In some embodiments, single cells are partitioned into individual discrete volumes for manipulation and analysis. In certain embodiments, such individual discrete volumes are droplets. In some embodiments, a first droplet encapsulates cells with a lysis buffer and reverse primers for genotyping, and a second droplet contains the first droplet contents along with the barcoding buffer, barcoding beads, and forward primers for genotyping. A kit of the disclosure may further include reagents for droplet formation.

In some embodiments, the system may further include a means for sorting and/or encapsulating individual cells into droplets. The means for sorting and/or encapsulating individual cells may include a microfluidic device.

Microfluidic devices (for example, fabricated in polydimethylsiloxane), generate sub-nanoliter reverse emulsion droplets. These droplets are used to co-encapsulate cells, cell lysates, proteins and/or nucleic acids with enzymes (e.g., polymerases, ligases, proteases), certain buffers (e.g., lysis buffers), and/or a bead harboring one or more bead-attached barcoding oligonucleotides, as described herein. Each bead, for example, can be uniquely barcoded (with bead-attached oligonucleotides degenerately barcoded between beads, e.g., where formed by successive rounds of split-and-pool oligonucleotide synthesis to generate distinct barcoded oligos upon each individual bead within a population of beads) so that each drop and its contents are distinguishable. The cells, cell lysates, transcriptomes and/or nucleic acids may come from any source known in the art. The cell can be lysed as it is encapsulated in the droplet. To load single cells and barcoded beads into individual droplets with Poisson statistics, 100,000 to 10 million such beads are needed to barcode about 10,000-100,000 cells.

In some embodiments, cells, cell lysates, and/or associated enzymes, beads, and/or other agents may be encapsulated in droplets, such as microfluidic droplets. Those of ordinary skill in the art will be aware of techniques for encapsulating particles within microfluidic droplets; see, for example, U.S. Pat. Nos. 7,708,949, 8,337,778, 8,765,485, or Int. Pat. Appl. Pub. Nos. WO 2004/091763 and WO 2006/096571, each incorporated herein by reference. In some cases, the particles may be encapsulated at a density of less than 1 particle/droplet (and in some cases, much less than 1 particle/droplet) to ensure that most or all of the droplets have only zero or one particle present in them. In other cases, the particles may be encapsulated at a density of 1, 2, 3, 4, 5, or more particles per droplet, and the location of multiple particles per droplet computationally determined after sequencing based on the presence of identical unique molecular identifiers associated with multiple particle barcodes.

In some embodiments, the system may further include reagents for PCR amplification. In some embodiments, each bead contains a unique barcode upon a bead-attached barcoding oligonucleotide (optionally, the bead-attached barcoding oligonucleotide is cleavable, allowing release from the bead, once encapsulated), and the droplet is incubated under conditions suitable for transferring the bead-attached barcoding oligonucleotide's bead-specific barcode to target genotyping amplicons and/or target RNA expression tracking constructs (or to amplicons generated therefrom).

### Other Methods

Also envisioned within the scope of the disclosure are methods for determining the presence of molecules in single cells using combinations of methods and assays as described herein.

In specific embodiments, single cells may be fixed before encapsulation and fixations and/or crosslinks may be reversed after encapsulation.

Any standard fixation methods known in the art may be used. Fixation of cells or tissue may involve but is not necessarily limited to, the use of methanol, the use of cross-linking agents, such as formaldehyde, and may involve embedding cells or tissue in a paraffin wax or polyacrylamide support matrix (Chung K, et al. Nature. 2013 May 16; 497(7449): 322-7). Standard methods for delivery of nucleic acid based probes to fixed cells may be used. Example methods for delivering to fixed cells may be found in U.S. Patent Application Publication No. 2017/0067096 A1, International Patent Application No. PCT/US2015/016788, and U.S. Patent Application Publication No. 2016/0305856 A1, each of which is incorporated herein by reference.

The targeting, sensing and/or genotyping probes are maintained under conditions sufficient to allow hybridization. Certain ligation dependent probes can then be ligated together. This may be done using any ligation technique commonly known in the art. Example standard ligation techniques may include, but are not necessarily limited to, ligation using SplintR, T4 DNA Ligase, T4 RNA ligase, or methods used in concatemer ligation assays, proximity ligation assays, and proximity extension assays such as those found in US 2016/0024572 A1 and PCT/US2014/028921.

In specific embodiments, 10 or more, preferably 50 or more probes are used. Specific embodiments include primer pairs for genotyping 10 or more, preferably 50 or more genomic loci, as well as probe/primer sets for monitoring expression of multiple different transcripts (e.g., 1 or more, 2 or more, 5 or more, 10 or more, 20 or more, or 50 or more).

In specific embodiments, the individual discrete volumes may be emulsion droplets or separate wells.

In specific embodiments, single cells may be lysed in oil emulsion droplets. Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be appropriate for the disclosure may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the disclosure, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the disclosure may be any specific or general polymerase known in the art and useful for the disclosure, including Taq polymerase, Q5 polymerase, or the like.

In certain embodiments, tagmentation is used to introduce adaptor sequences to genomic DNA in regions of accessible chromatin (e.g., between individual nucleosomes) (see, e.g., US20160208323A1; US20160060691A1; WO2017156336A1; and Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aabl601. Epub 2015 May 7). In certain embodiments, tagmentation is applied to bulk samples or to single cells in discrete volumes.

Methods for targeted RNA quantification in single cells may involve preparing droplets as described herein and delivering one or more targeting probes, sensing probes, etc. to the droplets and allowing hybridization, where each targeting probe includes, e.g., a UMI and a target binding region configured to hybridize to adjacent sites on a target RNA of interest. The cells or cell lysates may be resuspended in a ligase mix comprising an RNA-templated DNA ligase, wherein a circular DNA is generated when the target RNA of interest is bound by a probe. The cells or cell lysates may be resuspended in a PCR mix comprising primers specific to a reverse priming site, and the cells or cell lysates may be segregated into individual discrete volumes as described herein. Barcoded primers may be released from, e.g., beads via a cleavable linker, and PCR may be performed in the individual discrete volumes, generating cell-barcoded amplicons. The resulting amplicons may then be sequenced.

In specific embodiments, genotyping and/or RNA quantification may be done in single cells using a combination of any of the steps and methods described herein.

In certain embodiments, droplets are formed sequentially (e.g., via the Tapestri protocol). The first droplet encapsulates cells with the lysis buffer and reverse primers for genotyping. The second droplet contains the first round of droplets along with the barcoding buffer, barcoding beads, and forward primers for genotyping. The genotyping primers are designed, for example, via Tapestri DNA panel designer, all the other reagents are provided, for example, by the Mission Bio Tapestri Single-Cell DNA Sequencing Kit.

### Kits

The instant disclosure also provides kits containing agents of this disclosure for use in the methods of the present disclosure. Kits of the instant disclosure may include one or more containers comprising an agent and/or composition of this disclosure (e.g., a RNA expression tracking construct, a gene-specific probe, bead-attached barcoding oligonucleotides, etc.). In some embodiments, the kits further include instructions for use in accordance with the methods of this disclosure. In some embodiments, these instructions include a description of administration of the agent(s) and/or composition(s) to obtain paired expression level and genotype information.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for obtaining paired expression level and genotype information from single cells. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like.

Kits may optionally provide additional components such as enzymes (e.g., ligases, polymerases, etc.), buffers and interpretive information. Normally, the kit includes a container and a label or package insert(s) on or associated with the container.

Human genetics has rapidly advanced the discovery of mechanisms underlying a broad range of diseases. This is the case for both rare, highly penetrant genetic variants associated with Mendelian inheritance and more common variants linked with complex multigenic diseases that are affected by gene-environment interactions. The proliferation and expansion of disease-focused genetics consortia and biobanks has accelerated the process of discovery); however, there remains a significant bottleneck in translating these discoveries into identification of causal variants and their mechanisms of action. Continued efforts to interpret genome-wide association studies (GWAS) and derive novel hypotheses have benefited from integration of population genetics with disease atlases that incorporate gene expression and epigenetics. Thus, functional genomics has achieved notable success in uncovering thousands of disease-associated alleles and variants associated with expression quantitative trait loci (eQTL) through molecular profiling in cells and tissues derived from genetically diverse donors. Additional disease-associated eQTLs are emerging from single-cell transcriptomics and perturbational profiling, highlighting dynamic cell type-specific mechanisms of gene regulation that modulate discrete cellular phenotypes. Despite remarkable progress in uncovering genetic mechanisms of gene expression, a significant proportion of disease heritability remains unexplained. In this context, coding variants can impact protein function in a myriad of ways that are difficult to detect in eQTL studies. Additionally, noncoding variants are found in complex haplotype blocks that make establishing singular causality difficult. Therefore, there is an unmet need to devise systematic approaches for mechanistic characterization of variants, one at a time and on isogenic backgrounds in the relevant cell types. The advent of genome engineering has ushered in new opportunities for phenotyping variants. In particular, precise CRISPR-Cas genome editing tools, like base editors and prime editors, have advanced at a rapid pace and now enable installation of transition and transversion mutations as well as insertions and deletions. These base editors have been effectively deployed in pooled screening campaigns with tiled mutagenesis and individually to evaluate variants identified by GWAS. While editing technologies hold great promise, they often edit with varying efficiencies (0-95%) and precision (1-43 bp window) across different genomic loci and cell types, necessitating more accurate approaches for paired genotyping of installed edits with high content phenotypic readouts in the same cell. Current approaches to address this challenge are constrained by throughput and sensitivity. The STAG-seq process of the disclosure was developed to address these issues. The current methods combine amplicon-based targeted genotyping with probe-based hybridization of thousands of targeted transcripts. When combined in a simultaneous readout, these measurements enable highly sensitive detection of transcriptional states in cells where genotyping has confirmed desired editing outcomes without bystander effects, allowing for causal inference of small effects from base editing. STAG-seq was deployed herein with base editor screening to assign function to variants implicated in immune-mediated disease. Novel coding variants in inflammatory pathways were mechanistically characterized as were noncoding variants within a complex autoimmunity locus in primary human immune cells.

To enhance the utility of genetic association studies, it is essential to move beyond putative causal variants through statistical association and instead define the molecular mechanisms of disease-linked variants. The STAG-seq process of the instant disclosure, a droplet-based method for single-cell gDNA amplicon sequencing combined with targeted transcriptomics, was developed. STAG-seq allows for high sensitivity genotyping of hundreds of loci in parallel with transcriptomic measurements of thousands of genes across 10⁴-10⁵ cells in a single experiment.

Critically, combined with CRISPR base-editing, this allows for a new paradigm of experiments wherein variants can be introduced in a population of cells for simultaneous measurement of genotype at the edited site and gene expression to determine the effects of the variant on cellular function. Importantly, STAG-seq can quantify phenotypes of variants even when editing efficiency is very low and/or imprecise. Although base editing efficiencies in primary human cells can be high, they frequently occur at rates of less than 20%, and off-target bystander editing is a common occurrence with deaminase base editors. As genome engineering strategies continue to evolve rapidly, STAG-seq is well positioned to accommodate improved editors and advanced delivery systems into primary cells and tissues for phenotyping variants in high-fidelity disease models.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Primary Human Cells

Primary human CD34+ hematopoietic stem and progenitor cells (HSPCs) from mobilized peripheral blood of healthy donors were purchased from the Fred Hutchinson Cancer Research Center. Monocytes were isolated from buffy coats obtained from de-identified donors obtained from Research Blood components (Watertown, MA).

### Human Primary Monocyte Preparations

Monocytes were isolated from buffy coats using human Monocyte Enrichment Cocktail (StemCell Technologies, 15068) with Sepmate tubes (StemCell Technologies,85460) and ACK lysis buffer (ThermoFisher, A1049201) following the manufacturer's protocols. Monocytes were either edited directly or aliquoted and frozen at −80° C. in FBS with 10% DMSO for further use.

### Human Primary T Cell Preparations

PBMCs were first isolated by density gradient centrifugation with Ficoll-Paque™ (Cytiva, 17144003) and ACK lysis buffer (ThermoFisher, A1049201) following the manufacturer's protocols. Then CD4 or CD8 positive T cells were isolated from PBMCs using EasySep™ Human CD4+ or CD8+ T Cell Isolation Kit (StemCell Technologies, 15068) following the manufacturer's protocols. T cells were either activated directly or aliquoted and frozen at −80° C. in FBS with 10% DMSO for later use.

### Cell Cultures and Differentiation

### Primary Monocyte-Derived Macrophage Cultures

Monocyte-derived macrophages (MDMs) were cultured at the density of 5×10⁵/ml in macrophage medium (DMEM high glucose supplemented with 10% FBS [Sigma, F2442], 1% GlutaMAX [Life Technologies, 35050079], 1% penicillin/streptomycin [Life Technologies, 10378016], and M-CSF 100 ng/ml [Peprotech, 315-02]). Medium was changed every 2 days by replacing half of the volume with fresh medium. After 7 days of differentiation, MDMs were collected by TrypLE (ThermoFisher,12604039) and replated with fresh medium. After 24 hours of recovery, the cells were stimulated with TLR ligands or cytokines as follows: Pam3CSK4 (100 ng/ml, invivogene, tlrl-pms) for 4 hours; LPS (10 ng/ml, Invivogen, tlrl-pb5Ips) for 4 hours; IFN-γ (10 ng/ml, Peprotech, 300-02) for 6 hours, TGF-β1 (100 ng/ml, Peprotech, 100-21) for 6 hours; IL-1β (100 ng/ml, Peprotech, 200-01B) for 6 hours; IL10 (100 ng/ml, Peprotech, 200-10) for 6 hours. The cells were harvested with TrypLE for STAG-seq.

### HSPC Myeloid Differentiation

Primary human CD34+ hematopoietic stem and progenitor cells (HSPCs) from mobilized peripheral blood of healthy donors were purchased from the Fred Hutchinson Cancer Research Center. The cells were seeded in 6-well plates at a density of 1×10⁵/ml in StemSpan SFEM II medium (StemCell Technologies, 02690) with 1% L-glutamine (Thermo Fisher Scientific, 25-030-081) and 1% penicillin/streptomycin (Life Technologies, 10378016) and also cytokine supplements at different stages. The differentiation protocol was modified from previous reports. The cells were first cultured in medium supplemented with Flt3-L (100 ng/ml, Peprotech, 300-19), SCF (50 ng/ml, Peprotech, 300-07), IL-3 (5 ng/ml, Peprotech, 200-03), GM-CSF (5 ng/ml, Peprotech, 300-03), and G-CSF (3 ng/ml, Peprotech, 300-23) for 4 days to achieve optimal expansion of the HSCs and their differentiation towards the myeloid lineage. In the second step, the cells were cultured in medium supplemented with Flt3-L (100 ng/ml), SCF (50 ng/ml), IL-3 (5 ng/ml) and G-CSF (10 ng/ml) for 6 days to promote proliferation of the granulocytic lineage. In the last step, cells were cultured in medium with only G-CSF (50 ng/ml) for 7 days for further maturation of the neutrophils. Fresh media was added every two days to maintain the cell density below 5×10⁵/ml. The media was completely replaced on day 5 and 11, and cells were harvested at day 9 and day 17 for STAG-seq.

### Primary T Cell Cultures

After isolation, the CD4+ or CD8+ T Cells were cultured at a density of 0.5-1×106/ml in complete XVivol5 medium (Lonza, 02-053Q) supplemented with 5% fetal bovine serum, 50 μM 2-mercaptoethanol (Sigma, M3148), 10 mM N-acetyl 1-cysteine (Sigma, A7250), 1% penicillin/streptomycin (Life Technologies, 10378016). For CD4+ or CD8+ effector/memory differentiation, the medium was also supplemented with 10 ng/ml hIL-2 (R&D Systems, BT-002-050), 5 ng/ml of hIL-7 (R&D Systems, BT-007-AFL-025), 5 ng/ml hIL-15 (R&D Systems, BT-015-AFL-025) (Shy et al. Nat. Biotechnol., 41: 521-531). For CD4+ regulatory Th1 cell differentiation, the medium was supplemented with 10 ng/ml hIL-2, 5 ng/ml hIL-12 (R&D Systems, 10018-IL-010) and 1 μg/mL anti-IL4 (BioXcell, BE0240) (Hawkins et al. Immunity, 38: 1271-1284). T cells were activated with anti-CD3/CD28 Dynabeads at 1:1 ratio of the cells (ThermoFisher, 11131D) for 2 days in the complete XVivol5 medium with appropriate cytokines. After the activation, the Dynabeads were removed, the cells were edited by nucleofection and further cultured with the same complete medium with appropriate cytokines. Medium was changed every 2 days to keep the cell density below 1×10⁶/ml. The cells were cultured for 5 days (CD4+ and CD8+) or 7 days (Th1) before harvesting for STAG-seq.

### Cell Lines

THP-1 and Jurkat cells were cultured in T75 flasks between 2-8×10⁵ cells/mL in RPMI1640 medium supplemented with 10% FBS (Sigma, F2442), 1% GlutaMAX (Life Technologies, 35050079), 1% penicillin/streptomycin (Life Technologies, 10378016).

### Flow-Cytometry of HSPC-Derived Cells

At the indicated time points, cells were harvested by centrifugation at 400×g for 5 minutes and washed twice with FACS buffer (PBS with 1% FBS and 2 mM EDTA). Cells were then stained with conjugated antibodies diluted in 100 μL FACS buffer for 30 minutes on ice while protected from light. After incubation, cells were washed twice and resuspended with the FACS buffer for flow cytometry analysis.

| Vendor | Catalog | Antibodies |
|---|---|---|
| BD biosciences | 555724 | FITC, Mouse Anti-Human CD66b |
| BD biosciences | 563691 | BV650, Mouse Anti-Human CD16 |
| BD biosciences | 753537 | RY586 Mouse Anti-Human CD117 |
| Biolegend | 301815 | Pacific Blue, anti-human CD14 |
| Biolegend | 982506 | APC, anti-human CD14 |
| Biolegend | 371508 | PE/Cyanine7, anti-human CD11c |
| Biolegend | 303530 | Brilliant Violet 785, anti-human CD38 |
| Biolegend | 301830 | Brilliant Violet 421, anti-human CD14 |

Base Editor mRNA Plasmids

BE4max-NG, evoAPOBEC1-NG, and AB8e-SpRY plasmids were gifts from David Liu's lab. The near-PAMless SpCas9 SpRY domain was PCR amplified and Gibson assembly was used to replace the NG Cas9 domain with SpRY in BE4max and evoAPOBEC1 μlasmids using NEBuilder® HiFi DNA Assembly Master Mix (New England Biolabs, E2621L) according to the manufacturer's instructions. YE1 mutations (W90Y+R126E, PMID 28191901) were installed in BE4max and evoAPOBEC1 deaminase domains by PCR amplifying fragments of the plasmid using primers (IDT) encoding for each mutation. The same Gibson assembly method described above was used to generate IVT plasmids for each. The F148A mutation was installed in the ABE8e deaminase domain by PCR amplifying the plasmid using primers incorporating this mutation (IDT). To generate the TadCBEd(V106W)-SpRY IVT plasmid, Genscript synthesized the TadCBEd(V106W) deaminase domain and inserted it in place of evoAPOBEC1 in the evoAPOBEC1-SpRY IVT plasmid. The same protocol was used to generate eTdCBE-SpRY.

| Base editor | Source(s) |
|---|---|
| BE4max-NG | BE4max architecture |
| | rAPOBEC1 deaminase |
| | NG Cas9 domain |
| BE4max-SpRY | SpRY Cas9 domain |
| BE4max(YE1)-NG | NG Cas9 domain |
| | YE1 |
| BE4max(YE1)-SpRY | YE1 |
| AB8e-SpRY | AB8e deaminase |
| | SpRY Cas9 domain |
| AB8e(F148A)-SpRY | F148A |
| evoAPOBEC1-NG | |
| evoAPOBEC1-SpRY | evoAPOBEC1 |
| evoAPOBEC1(YE1)-NG | YE1 |
| evoAPOBEC1(YE1)-SpRY | YE1 |
| eTdCBE-SpRY | |
| TadCBEd(V106W)-SpRY | |

In Vitro Transcription of Base Editor and Prime Editor mRNA

Base editor-encoded mRNA was transcribed in vitro with the protocol described previously. Briefly, the base editor cassettes (ORFs) were cloned into a plasmid containing an inactive T7 (dT7) promoter, 5′ untranslated region (UTR), Kozak sequence, and 3′ UTR (a gift from David Liu's lab). All the components were PCR amplified with Q5® High-Fidelity 2× Master Mix (New England Biolabs, M0492L) using a forward primer that corrects T7 promoter sequence and a reverse primer that installed the poly(A) tail (11 9 bp). The PCR products were gel purified and served as templates. mRNA was generated using the HiScribe T7 High-Yield RNA Kit (New England Biolabs, E2040S) according to the manufacturer's instructions, with co-transcriptional capping by CleanCap AG (TriLink Biotechnologies) and full replacement of UTP with N1 Methylpseudouridine-50-triphosphate (TriLink Biotechnologies). The Transcribed mRNAs were precipitated with 2.5 M lithium chloride (Thermo Fisher Scientific) and washed twice in 70% ethanol, then reconstituted in IDTE buffer (10 mM Tris, 0.1 mM EDTA, pH 8.0 at 25° C.). mRNAs were QC tested with Tapestation system (Agilent) and quantified with NanoDrop One UV-Vis spectrophotometer (Thermo Fisher Scientific), then diluted to 2 μg/μl and stored at −80° C. for further use.

Variant Generation with Guide RNA and Base Editor mRNA Electroporation

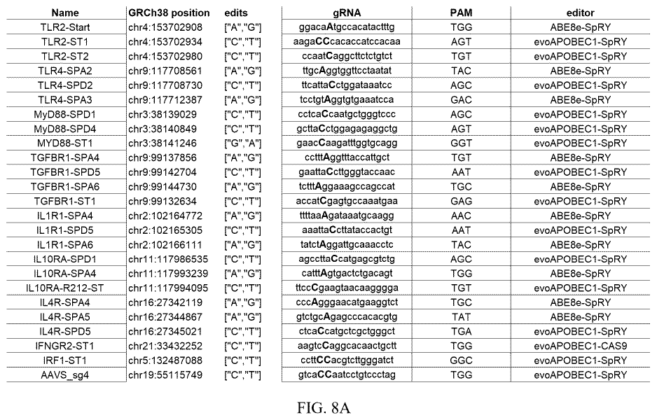

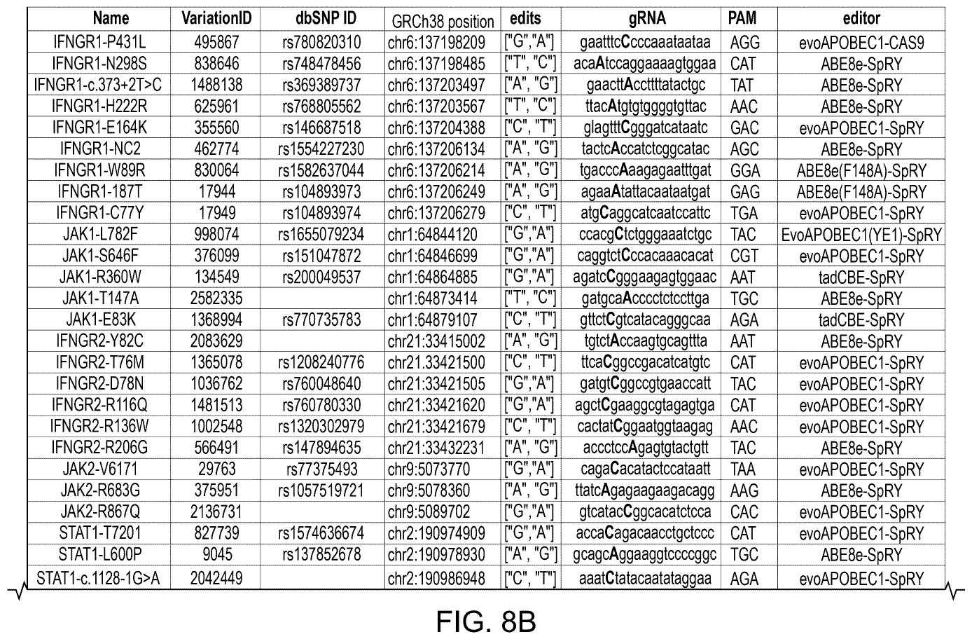

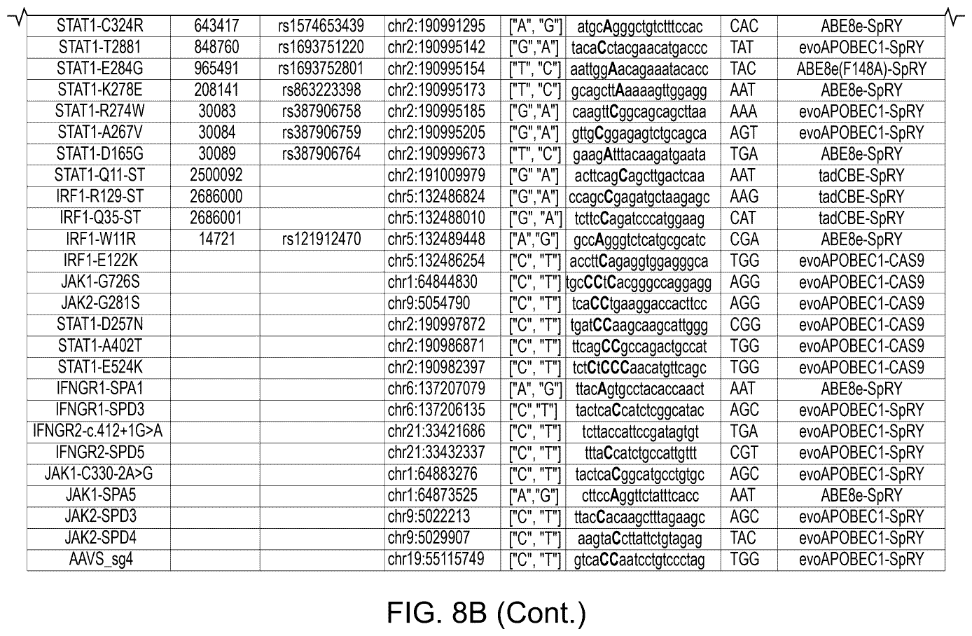

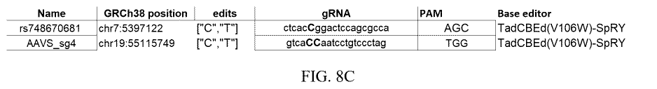

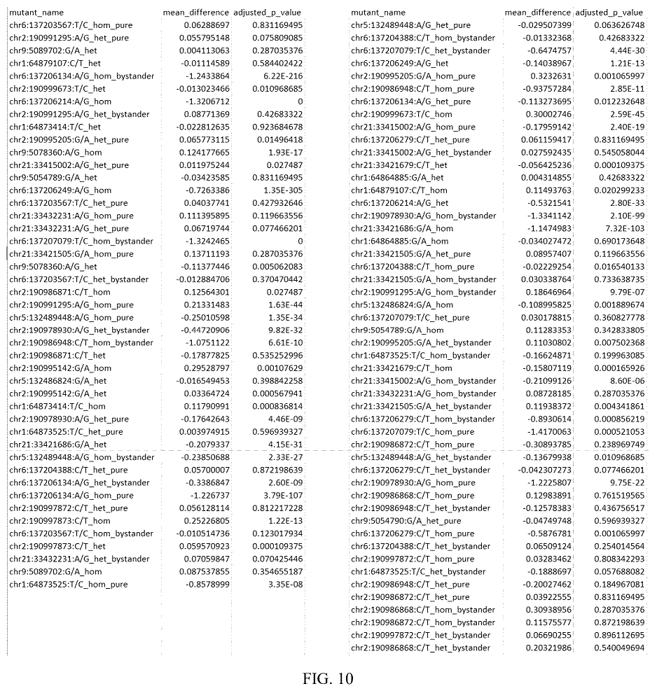

Cells were washed three times in DPBS with 0.1% BSA, and then resuspended at 2×10⁵/μl (Monocytes) and 2×10⁴/μl (HSPCs and iPSC) in 10 μl P3 Lonza buffer with supplement (Lonza, V4XP-3032). The editing master mix was prepared by combining 1.5 μl base editor mRNA (2 μg/μl) and 1.5 μL of sgRNA (100 μM in IDTE pH 7.5, IDT) (FIGS. 8A-8C), 10 μl P3 Lonza buffer with supplement. The cells were gently mixed with the master mix and transferred to the 20 μl electroporation cuvette (Lonza, V4XP-3032). Cells were electroporated using the program CM-137 (monocytes) and DS-130 (HSPC) program in the 4D-Nucleofector X Unit. Immediately after electroporation, 120 μl prewarmed appropriate media was added to the cuvette. The cuvette was placed in a 37° C. incubator for 20 min to allow the recovery of the cells. Cells were then seeded at a density of 5×10⁵ M/ml (monocytes), 2×10⁵ M/ml (HSPCs) in the appropriate complete medium.

Gene Knockout with RNP Electroporation in T Cells

T cells were prepared as above in 10 μl P3 Lonza buffer. The editing master mix was prepared by combining 1.2 μL of sgRNA (100 μM in IDTE pH 7.5, IDT) with 1 μl S.p. HiFi Cas9 Nuclease (IDT, 1081061). The master mix was incubated at room temperature for at least 30 min, then diluted in 10 μl P3 Lonza buffer. The cells were then electroporated and recovered as above.

### Genotyping by Sanger Sequencing and NGS

Edited cells were genotyped on day 7 after editing. gDNA was extracted from 1 million cells using QuickExtract™ DNA Extraction Solution (LGC Biosearch Technologies, QE09050) according to manufacturer's instructions. Briefly, 1 million cells were washed once in PBS, then resuspended in 200 μL DNA Extraction Solution. The mixture was incubated at 65° C. for 20 minutes, then lysed at 98° C. for 5 minutes.

Edited regions of the genome were amplified from 3 μL lysate using GoTaq® 2X Master Mix (Promega M7132) supplemented with 1M betaine (Sigma 61962-50G) according to manufacturer's instructions. Products were purified using AMPure XP reagent (Beckman-Coulter A63881) according to manufacturer's instructions and eluted into nuclease-free water.

Sanger sequencing was performed through Genewiz from Azenta Life Sciences. The editing efficiencies were predicted using the Moriarty lab's EditR software.

NGS sequencing was performed through the Massachusetts General Hospital Center for Computational and Integrative Biology DNA Core using their CRISPR Sequencing service. Primers were designed to position the base editing site within the first 100 bp of the short amplicon, and the region of interest was PCR amplified using the above GoTaq protocol. The PCR products were purified as above using AMPure XP Reagent and eluted into IDTE buffer (10 mM Tris, 0.1 mM EDTA). PCR products were diluted to between 10-40 ng/μL in 35 μL total volume, then sent for sequencing, where NGS adaptors and unique barcodes were added ahead of sequencing.

### Design of STAG-Seq Probes

The STAG-seq probes were designed as described previously with some modifications. The Primer Binding Site of the 5′ probe was replaced with a sequence including the reverse complements of the Tapestri barcoding oligo: 5′-CGAGACTACTGCGAGTAC-3′ (SEQ ID NO: 1). To select the target sequences (52 bp) of RNA, all the previously published guidelines were followed. In order to deal with variable expression of splice variants, the exon of each gene was ranked by normalized exon detection frequency in the GTEX human whole blood data set, then the probes that map to the top expressed exons with 1 probe per exon were selected. The structure of the probe:

5′ Probes:
(SEQ ID NO: 2)
/5Phos/GGAGGGCAGCAAACGGAA-(25 bp transcript
specific sequences)-NNNNNNNNNNCGAGACTACTGC
GAGTAC
3′ Probes:
(SEQ ID NO: 3)
(25 bp transcript specific sequences)-TAG
AAGAGTCTTCCTTTACG.

Sequences designed for STAG-Seq

a. The HyPR probe sequences for specific RNA detection:
5′ probes: /5Phos/GGAGGGCAGCAAACGGAA(25 bp of DNA sequences that is reverse complement to targeted RNA)NNNNNNNNNNCGAGACTACTGCGAGTAC (SEQ ID NO: 4)
3′ probes: (25 bp of DNA sequences that is reverse complement to targeted RNA) TAGAAGAGTCTTCCTTTACG (SEQ ID NO: 5)

The 25 bp sequences complementary to the targeted RNA in the 5′ and 3′ probes are located 2 bp apart in the RNA.

b. Hairpin oligo to stabilize probe/RNA structure:

(SEQ ID NO: 6)
CGTAAAGGAAGACTCTTCCCGTTTGCTGCCCTCCTCG
CATTCTTTCTTGAGGAGGGCAGCAAACGGGAAGAG

c. Readout oligo to enhance the detection specificity:

(SEQ ID NO: 7)
CTTACGGATGTTGCACCAGCAAGAAAGAATGCGA

d. Tapestri barcoding oligo, which is provided on the beads of Tapestri kits:

(SEQ ID NO: 8)
tcgtcggcagcgtcagatgtgtataagagacagnnnnnnnnn
VVVagtaTgtacgagtcnnnnnnnnngtactcgcagtagtc

e. Biotin-readout oligo, employed to selectively pull down the HyPR library, enables the separation of DNA and RNA libraries.

f. The indexing oligo for RNA library:

P7:
(SEQ ID NO: 9)
CAAGCAGAAGACGGCATACGAGATNNNNNNNNCAAGCAGAAGA
CGGCATACGAGATTAAGGCGAGTTGGCACCAGGCTTACGGATG
TTGCACCAGC
P5:
(SEQ ID NO: 10)
AATGATACGGCGACCACCGAGATCTACACNNNNNNNNAATGAT
ACGGCGACCACCGAGATCTACACGCTACGCTTCGTCGGCAGCG
TCAG

g. Oligo for sequencing:

Custom Read 1:
(SEQ ID NO: 11)
TCGTCGGCAGCGTCAGATGTGTATAAGAGACAG
Custom Index 1:
(SEQ ID NO: 12)
GCTGGTGCAACATCCGTAAGCCTGGTGCCAAC
Custom Index 2:
(SEQ ID NO: 13)
CTGTCTCTTATACACATCTGACGCTGCCGACGA
Custom Read 2:
(SEQ ID NO: 14)
AGCAAGAAAGAATGCGAGGAGGGCAGCAAACG

### Probe Pool Phosphorylation

The STAG-seq probes were ordered via IDT DNA oligo pools services. The 5′ probe pools were phosphorylated in a 100 μl reaction containing: 2000 pmol oligos, 10 μl of T4 ligase buffer, 3 μl of T4 PNK enzyme, 0.3 μl ATP (100 mM) and remaining volume of water. The reactions were incubated at 37° C. (phosphorylation) for 1 hour, followed by incubating at 65° C. for 20 min to heat-inactivate T4 PNK. The oligos were precipitated in −20° C. overnight with 1/10th the volume of 3 M sodium acetate (pH 5.2) and an equal volume of isopropanol. After washing with ice-cold 80% ethanol, the oligos were resuspended at final concentration of luM for further use.

### DNA Amplicon Panel Design

The DNA panels were designed through Mission Bio's Tapestri Designer platform with GRCh38 genome. Each DNA panel contains the oligo pairs that generate 49 to 312 non-overlapping amplicons (175-275 bp) detecting the variant and control loci.

### STAG-Seq Experimental Protocol

### Cell Preparation

The protocol described here is for 1 M cells. For MDMs, supernatant was removed and cells were trypsinized at 37° C. for 5 min, then harvested and washed in 5 ml cold PBS/0.1% BSA, and finally spun at 350 g for 5 min at 4° C. After resuspending the cells in lml cold PBS, 4 ml of −20° C. prechilled MeOH with 1.25 mg/ml DSP (dithiobis(succinimidyl propionate)(Thermo Scientific™, 22585) was added while gently vortexing the cells. Cells were fixed on ice for 1 h. At this point, cells can either be stored at −80° C. for a couple of weeks, or be used immediately. Cells were spun down at 800×g and rehydrated in 5 ml of 3×SSC (Thermo Scientific™, AM9765) containing 0.2% Tween and 40 mM Tris-HCl PH7.5 (3×SSCTT) and incubated at room temperature for 15 min to quench the DSP. Cells were then spun at 600×g at room temperature. Cells were washed with 3×SSCTT another two times, and resuspended in 5×SSC containing 0.2% Tween (5×SSCT). Cells were collected in 2-ml low-DNA binding round-bottom tubes, resuspended in 500 μl hybridization buffer (5×SSC, 30% formamide, 0.1% Tween 20) and incubated at 37° C. for 5 min. The prehybridized cells were centrifuged, resuspended in 100 μL Probe Mix (prepared by pooling probe sets in probe hybridization buffer to a final concentration of 20 nM per probe), and incubated for at least 24 h at 37° C. in a hybridization oven (VWR Cat #10055-006).

After hybridization, cells were harvested and washed in 500 μL probe hybridization buffer by incubating for 15 min at 37° C. The wash step was repeated three additional times, for a total of four washes. During the washes, the hairpin was prepared by snapcooling 2 μL (per reaction) of 3 μM H1B1 (CGTAAAGGAAGACTCTTCCCGTTTGCTGCCCTCCTCGCATTCTTTCTTGAGGAGGGC AGCAAACGGGAAGAG (SEQ ID NO: 15), Molecular Instruments) stock in hairpin storage buffer; reactions were heated at 37° C. for 2 min and slowly cooled in the PCR machine over 30 min to promote hairpin formation. After the final wash in probe hybridization buffer, cells were resuspended in 500 μL 5×SSCT and incubated at room temperature for 5 min. Cells were centrifuged for 5 min and resuspended in 100 μL 75 nM cooled B1H1 hairpin in 5×SSCT, then incubated at room temperature for 2 h. After the B1H1 hairpin hybridization, the cells were washed twice with 500 μL 5×SSCT, then resuspended in 200 μL 5×SSCT with 75 nM readout oligo. The cells were incubated at room temperature for 1 hour. After that, cells were washed three time with 5×SSCT, one time with 200 μl 1× T4 ligase reaction buffer without DTT (Dilute from 10× T4 ligase reaction buffer: 500 mM Tris-HCl, 100 mM MgCl2, 10 mM ATP, pH 7.5), then resuspended in 200 μL 1× ligase (1×T4 ligase buffer without DTT, 1:50 dilution of T4 DNA ligase, New England Biolabs #M0202S) and incubated at room temperature for 2 h. Following ligation, cells were washed three times in 500 μL 1× PBST and passed through a 20-μm filter (20 m pluriStrainer cat. 43-0020-01). The cells were then counted using a hemocytometer Counting Chamber (VWR, 76299-416) and checked for single-cell suspension.

### In House Probe Synthesis and Preparation

For the 50K probe set, 5′ and 3′ probes were synthesized as separate oligonucleotide pools using Twist Bioscience's oligo pool synthesis service. Oligo pools were diluted and pre-amplified by PCR, then the pool was transcribed into RNA, followed by reverse transcription of the RNA back to single-strand probes.

### DNA Amplicon Panel Design

The DNA panels were designed through Mission Bio's Tapestri Designer platform with GRCh38 genome. Each DNA panel contains the oligo pairs that generate 49 to 312 non-overlapping amplicons (175-275 bp) detecting the variant and control loci.

### Generation of Emulsions Through Tapestri

Hybridized Cells were centrifuged at 400×g for 5 min and resuspended in Cell buffer (Tapestri kit, Mission Bio) at 3000 cells/ul. Then the cells were encapsulated with a lysis buffer following the Tapestri Single-Cell DNA sequencing v3 User Guide. The lysis buffer, which includes proteases, is provided commercially in the Mission Bio Tapestri Single-Cell DNA Sequencing kit. After encapsulation, the cells were digested in the lysis emulsion with the PCR program: 50° C. 90 min, 80° C. 10 min, 4° C. hold. The lysis emulsion was handled following the Tapestri protocol to barcode the cells. The following cycling conditions were used: Denaturation: 98° C. for 6 min; Cycling 1: 11 cycles of 95° C. for 30 s, 72° C. for 10 s, 61° C. for 6 min, and 72° C. for 20 s; Cycling 2: 12 cycles of 95° C. for 30 s, 72° C. for 10 s, 48° C. for 6 min, and 72° C. for 20 s; final extension: 72° C. for 5 min. All PCR cycling steps were performed with the ramp rate at 1° C./s. After the targeted PCR amplification in the droplets, the barcoding emulsion was broken with the extraction agent and the top aqueous layer was diluted with total 320 μl water and carefully retrieved into a low-binding 1.5EP tube. The sequential droplet formation process described herein followed the Tapestri protocol. The first droplet encapsulated cells with the lysis buffer and reverse primers for genotyping. The second droplet contained the first round of droplets along with the barcoding buffer, barcoding beads, and forward primers for genotyping. The genotyping primers were designed via Tapestri DNA panel designer, all the other reagents were provided in the Mission Bio Tapestri Single-Cell DNA Sequencing Kit.

### Isolation of Probe Library and Genomic DNA Library

288 μl SPRI beads (Beckman, B23318) (0.72×) was added to the sample, which was then incubated at room temperature. Beads were magnetically separated with gDNA amplicons and supernatant was collected for further probe library enrichment.

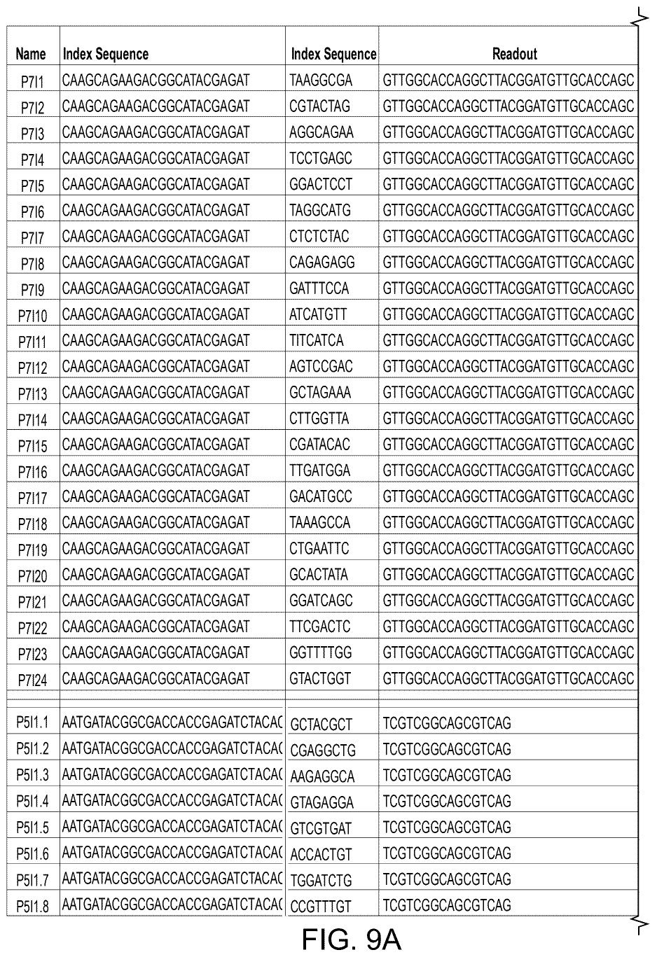

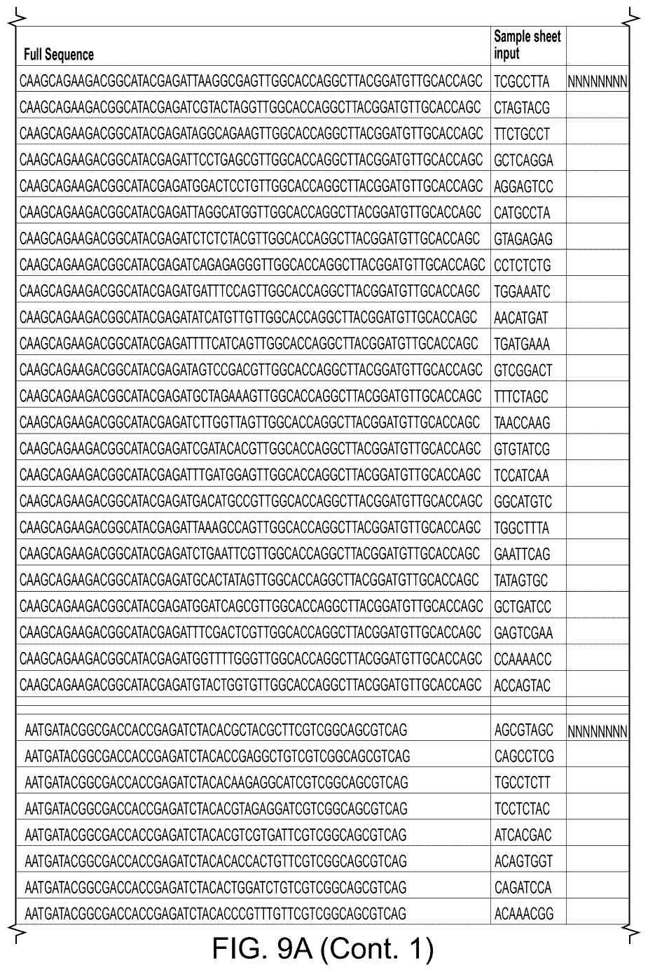

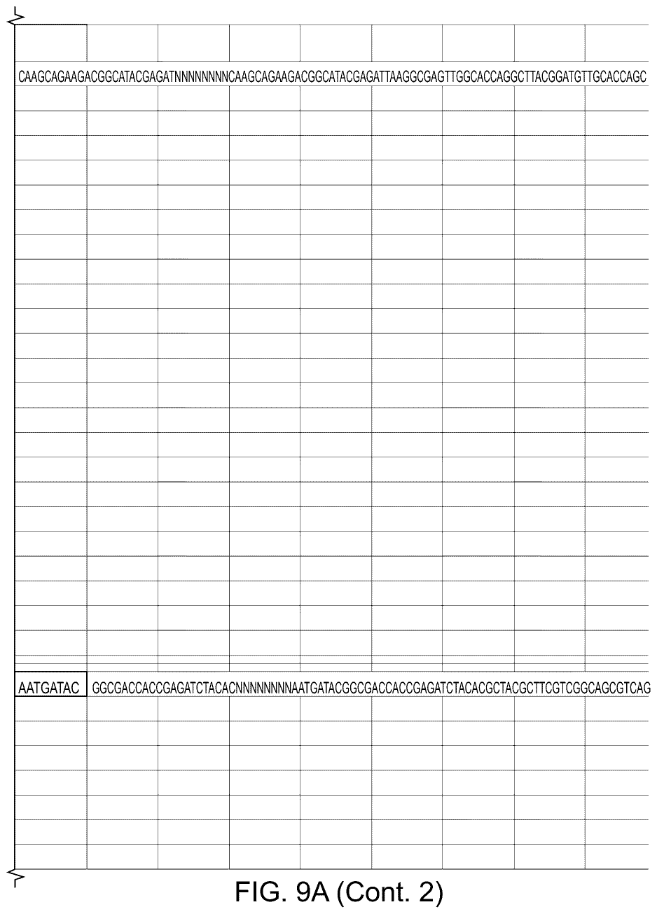

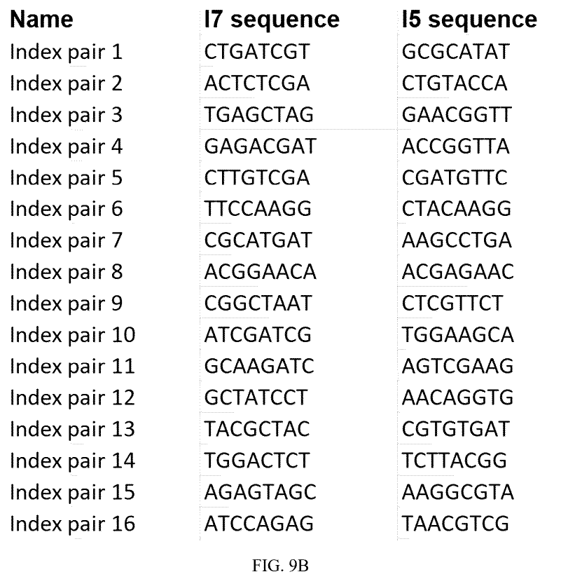

For the gDNA amplicons, beads were washed with 80% EtOH twice, eluted in 100 μl of water, then further cleaned up with 80 μl SPRI beads (0.8×), and eluted in 60 μl of water. Library PCR was performed following Tapestri Single-Cell DNA sequencing v3 User Guide (FIGS. 9A-9B).

For the probes, 4 μl of 5 μM Biotin-readout oligo (cttacggatgttgcaccagcaagaaAAAAAAAAAA/3Bio/(SEQ ID NO: 16)) was added and the sample was incubated at 96° C. for 10 minutes, then transferred on ice for 10 min. Further pull down of probe library was performed using 80 μl Dynabeads™ Streptavidin (Thermo Fisher Scientific, 65605D) following manufacturer's instruction, and beads were resuspended in 50 μl of water. For the final probe library, 15 μl of beads were transferred into a 50 μl PCR reaction following the cycling conditions: 95° C. for 3 min; Cycling: 14 cycles of 98° C. for 20 s, 63° C. for 40 s, and 72° C. for 30 s; final extension: 72° C. for 5 min (FIGS. 9A-9B). The PCR products were loaded on a gel to extract the band around 260 bp (Zymo, #D4008) and eluted in 15 μl water.

### Sequencing

The gDNA Libraries were loaded at a concentration of 750 nM with 7% Phix on Nextseq 2000, sequenced using a 150 base pair (Read 1), 8 base pair (Index 1), 8 base pair (Index 2), 150 base pair (Read 2) configuration. The depth of sequencing scaled with the number of gDNA amplicons. The probe libraries were loaded at a concentration of 750 nM without Phix on Nextseq 2000 with either of the following two configurations: 1) 140 base pair (Read 1), 8 base pair (Index 1), 8 base pair (Index 2), 0 base pair (Read 2) or 2) custom primers, using a 53 base pair (Custom Read 1: TCGTCGGCAGCGTCAGATGTGTATAAGAGACAG (SEQ ID NO: 11)), 8 base pair (Custom Index 1: GCTGGTGCAACATCCGTAAGCCTGGTGCCAAC (SEQ ID NO: 12)), 8 base pair (Custom Index 2: CTGTCTCTTATACACATCTGACGCTGCCGACGA (SEQ ID NO: 13)), 40 base pair (Custom Read 2: AGCAAGAAAGAATGCGAGGAGGGCAGCAAACG (SEQ ID NO: 14)) configurations. All custom primers were added according to the manufacturer's instructions. The depth of sequencing scaled with the number of genes, typically aiming to sequence 10 reads per probe per cell.

### STAG-Seq Computational Pipeline

### Quantification of RNA-Transcript Abundances

After demultiplexing the HyPR-Seq probe library, the raw.fastq files were processed into unique molecular identifier (UMI) count matrices using a modified HyPR-seq pipeline, optimized for the STAG-seq read structure. The pipeline extracts the cell barcode from read 1 and the UMI+ probe from read 2. The unique 3-tuples are then counted into an N_barcode×N_probe count matrix. In each cell, to compute RNA-transcript abundance for a specific gene, we sum the UMI counts for all probes targeting that gene. This produces a N_barcode×N_genes matrix where each entry represents the number of UMIs detected for a specific gene in a given cell.

### Processing Genotype Information

After demultiplexing gDNA libraries, raw .fastq files were processed into .loom files using the Tapestri pipeline version 3 provided by MissionBio through their web portal. This pipeline uses both a reference genome and an amplicon panel file, which are available at the Gene Expression Omnibus (GEO) series.

The Tapestri pipeline performs cell calling using a correlation UMAP algorithm to cluster valid and invalid cell barcodes. This approach is described in detail in MissionBio's documentation: support.missionbio.com/hc/en-us/articles/360042381634-Step-5-Cell-calling.

After cell calling, the pipeline genotypes individual cells with the Genome Analysis Toolkit (GATK). More information on this variant calling is available in MissionBio's documentation: support.missionbio.com/hc/en-us/articles/360042391554-How-does-Pipeline-callgenotypes.

### Data Integration, Donor Separation, and Preprocessing

To integrate and process the data from HyPR-seq and Tapestri platforms, custom Python scripts developed for STAG-Seq analysis were employed. These scripts were built upon the Scanpy, AnnData, and h5pylibraries libraries. Initially, count matrices from HyPRseq and loom files from Tapestri were loaded into AnnData objects. Cells from the HyPR-seq dataset were then subjected to quality control by applying a minimum UMI threshold, determined by manually identifying the inflection point (“knee”) of the cell barcode rank plot. Subsequently, barcodes from HyPR-seq that passed this filter were intersected with those from Tapestri for each sample, ensuring that each transcriptomic profile was accurately paired with its corresponding genotype within the same cell. Since both the HyPR-seq and Tapestri pipeline independently filter invalid barcodes, the intersection of valid cells is generally >80%.

In certain cases, cells from multiple donors were pooled together in a sample and sequenced. To cluster the donors, a normalized genotype profile for each cell was computed by dividing the number of reads with evidence for a given variant divided by the sum of all reads with and without evidence of that variant+1 (to avoid 0/0). Clustering was performed via the k-means algorithm implemented in the sklearn library with the parameter n_clusters set to 2, reflecting the maximum number of donors (two) in any given experiment.

For normalization of transcriptional profiles, each cell's total UMI count was scaled to 10,000 UMIs using the scanpy.pp.normalize_total function. This was followed by logarithmic transformation with the scanpy.pp.log 1p function to stabilize the variance across cells.

In the case where cell types are heterogeneous within an individual sample, cell types were annotated by clustering transcriptional profiles. Principal component analysis was first performed with all genes using scanpy.pp.pca and then the cell-cell adjacency matrix was computed with scanpy.pp.neighbors using default parameters. Then the Louvain clustering algorithm implemented with scanpy.tl.louvain function with a resolution of 0.55 was run. Each cluster's marker genes were identified with the scanpy.tl.rank_gene_groups function and those marker genes were used to annotate each cluster alongside literature knowledge of known cell-type markers.

### Identifying Mutants and Differential Expression Analysis

To classify genotyped variants within our dataset, cells exhibiting exactly one variant from our targeted set of variants were classified based on the genotype call provided by GATK, denoting them as either homozygous or heterozygous for that variant. Additionally, cells were annotated as having a bystander mutation if another nongermline heterozygous or homozygous single nucleotide polymorphism (SNP) was detected within 10 base pairs of the targeted genomic edit.

For differential expression analysis between a variant and a reference genotype, cells harboring the specific variant of interest were compared against cells that underwent adeno-associated virus (AAV) safe harbor edits. This analysis was conducted using Scanpy's scanpy.tl.rank_gene_groups function, which implements the Wilcoxon ranksum test. With n_genes hypotheses tested, the Benjamini-Hochberg procedure was employed to calculate adjusted p-values given a target false discovery rate (FDR) of 0.05. In the cases where DE pertaining to stimulation-response was computed, the population of stimulated WT (unedited) cells was compared to unstimulated WT cells with the same approach implementing Wilcoxon rank-sum test.

### IFN-γ Response Score

A set of genes was identified that reflected the transcriptional response to IFN-7.

Differential expression analysis was performed between IFN-γ treated and PBS treated cells with scanpy.tl.rank_gene_groups, using only cells where no target variants were identified. Genes were ranked based on their log-fold change (log FC) between IFN-γ stimulation and PBS, extracting the 100 highest log FC genes (positive response genes) and 100 lowest log FC genes (negative response genes).

For each IFN-γ stimulated cell, we compute an “IFN-γ response score” as the difference between the mean expression of positive response genes and the mean expression of negative response genes. We then use the Wilcoxon rank-sum test to compare the IFN-γ response score of cells with a specific genetic variant to wild-type cells without targeted variants (unedited cells). With n_variants hypotheses tested, the Benjamini-Hochberg procedure was employed to calculate adjusted p-values given a target false discovery rate (FDR) of 0.05.

To report an estimate of the mean impact of each variant on IFN-γ response across all cells with that variant, the mean IFN-γ response score of the unedited cells was subtracted from the mean IFN-γ response score of the cells with a given variant. Then, the resulting scores across all variants considered were z-scored. As such, if the previously introduced rank-sum test resulted in statistical significance for a given variant, a positive normalized score supports a gain-of-function variant classification, whereas a negative normalized score supports a loss-of-function variant classification (FIG. 10).

### TNRC18 Pseudotime Analysis

The CytoTRACE algorithm implemented via the CellRank package was run on expression profiles of cells from both day 9 and day 17 timepoints. Cells from the neutrophil lineage were extracted and ordered by their calculated CytoTRACE time. A 1-D smoothing spline was fit to the log normalized TNRC18 expression across pseudotime using the scipy.interpolate.UnivariateSpline function.

### Variant Effect Power Analysis

Power analyses of how STAG-seq would perform at downsampled UMIs and in scenarios where fewer cells with a specific variant are measured were performed. In our simulation, our UMI/cell was downsampled to a target level and subsampled to a targeted number of variant cells. Differential gene expression was then performed as described in the above section Identifying Variants and Differential Expression Analysis

Recovered differentially expressed genes (DEGs) were computed as the intersection of the simulated DEG and the DEG recovered in the ideal scenario of maximum UMI/cell (5,500) and number of variant cells (44 and 127 for STAT1-L600P and IFNGR2-Y63C respectively). For each targeted UMI/cell and mutant count, the simulation is repeated 3 times and take the average DEG recovered. The mean DEG recovered for each UMI/cell target and variant number was plotted as a heatmap.

A simulation of how STAG-seq's ability to identify mutants by single cell genotyping compares with inferring the presence of a mutant by expression of individual guide RNAs was performed. Given a fixed number of homozygous mutants (44 and 127 for STAT1-L600P and IFNGR2-Y63C), the theoretical number of unedited and heterozygous cells that would mix with that number of homozygous cells for various editing efficiencies was calculated, as well as the total number of cells that would be required per variant. If the editing efficiency is P_{edit} and targeted homozygous cell count is N_{homozygous}, the theoretical number of unedited and heterozygous cells is calculated as follows:

$${N}_{t o t a l} = \frac{{N}_{h o m}}{{P}_{{edit}^{2}}} , {N}_{h e t} = {N}_{t o t a l} \times 2 ( {p}_{e d i t} ) ( 1 - {p}_{e d i t} ) ,$$

$${N}_{u n e d i t e d} = {N}_{t o t a l} \times {( 1 - {p}_{e d i t} )}^{2}$$

For each editing efficiency, N_het cells from observed heterozygous mutants and N_unedited from observed wild-type cells were sampled. Then differential gene expression analysis was performed and the number of DEG recovered by intersecting the DEGs from the simulated scenario with those recovered in the ideal scenario of STAG-seq where the mixture contains only homozygous mutant cells was reported. To create the plot, the DEG recovered and total cells required over 5 simulations for each editing efficiency was averaged. The DEG recovered was plotted, as well as the total cell requirement for each editing efficiency for STAT1-L600P and IFNGR2-Y63C, respectively.

### Additional Statistical Analysis

Other statistical analyses were performed using Prism (GraphPad Software) or Excel (Microsoft office) to generate curves or bar graphs. All error bars represent standard error of the mean (SEM). Two-tailed unpaired t-test was used for statistical analysis of two groups of samples. One-way ANOVA analysis of variance with a Newman-Keuls post-test was used to evaluate statistical significance of multiple groups of samples. *p<0.05; **p<0.01; ***p<0.001. p>⁰0.0⁵ was considered not significant (NS).

### Example 2: STAG-Seq Enabled Sensitive Profiling of Genomic DNA Variation and RNA Expression in Single Cells

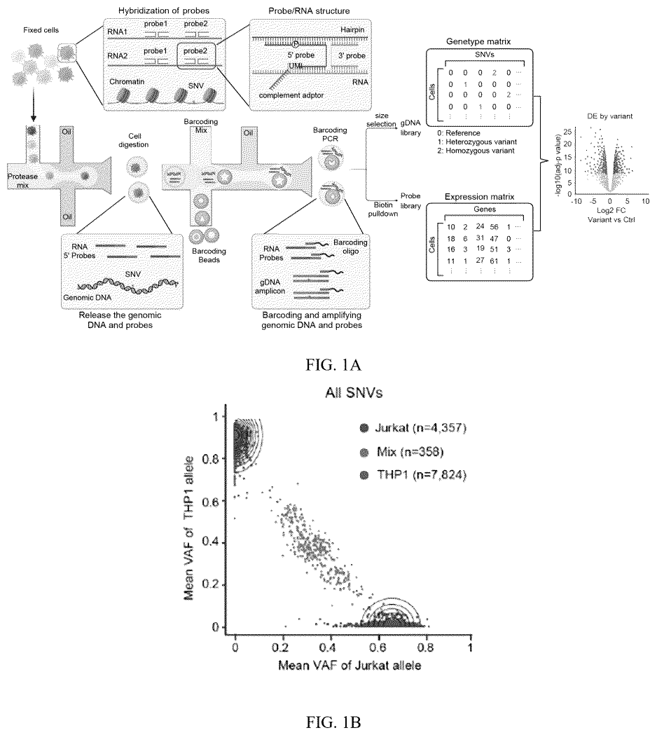

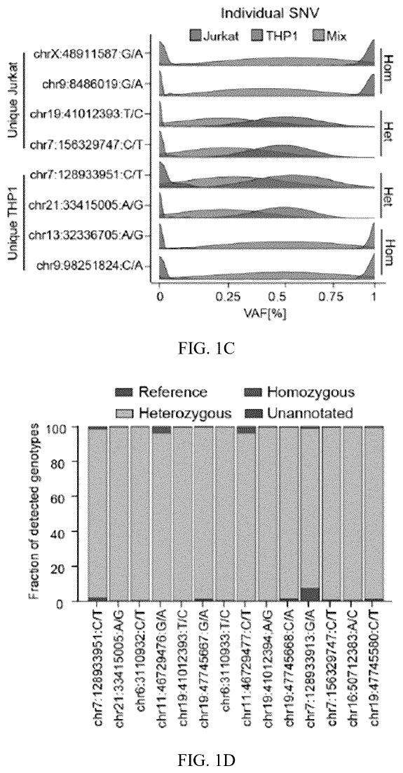

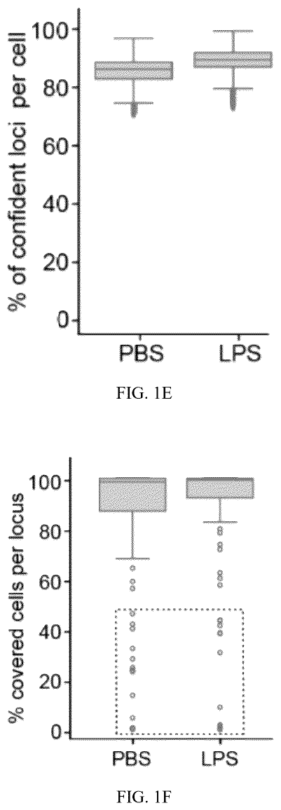

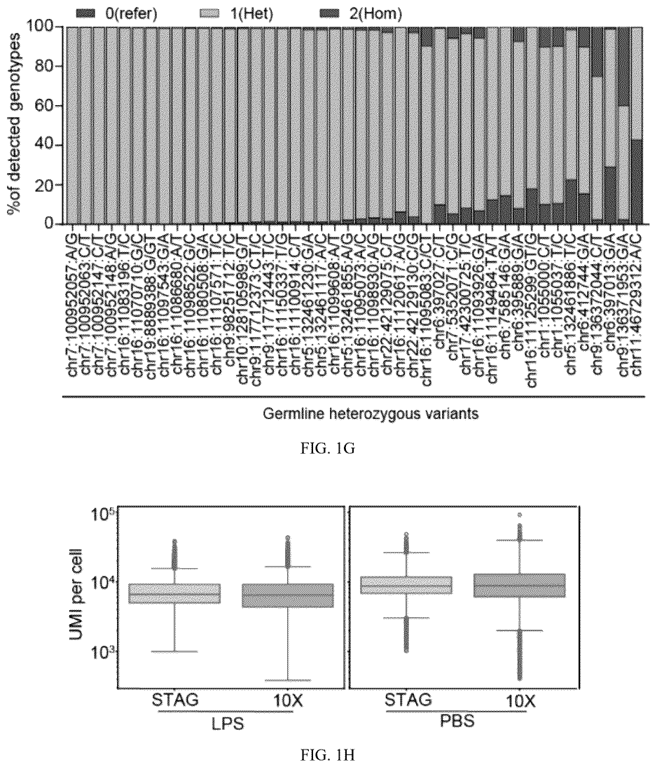

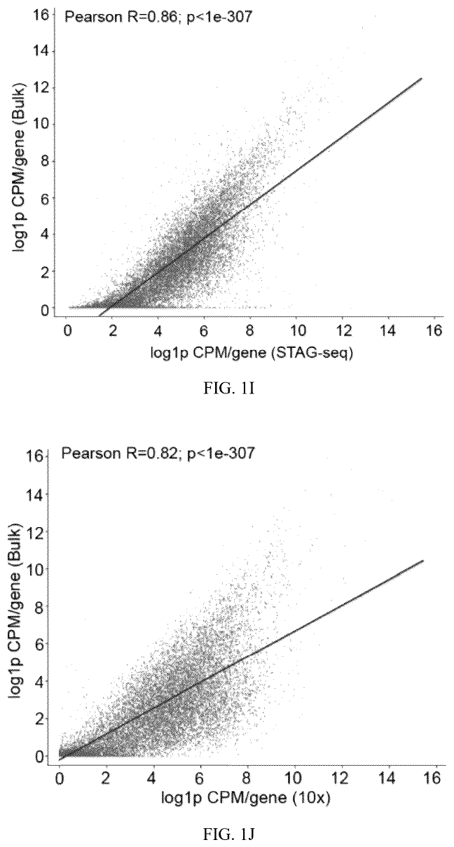

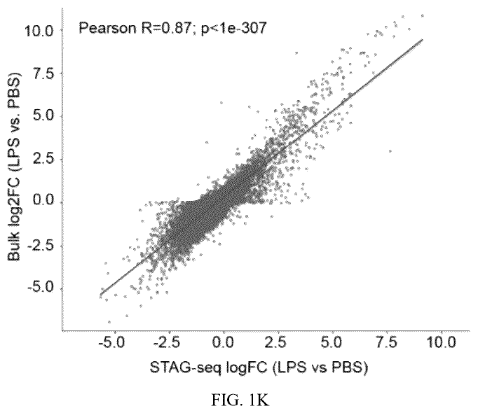

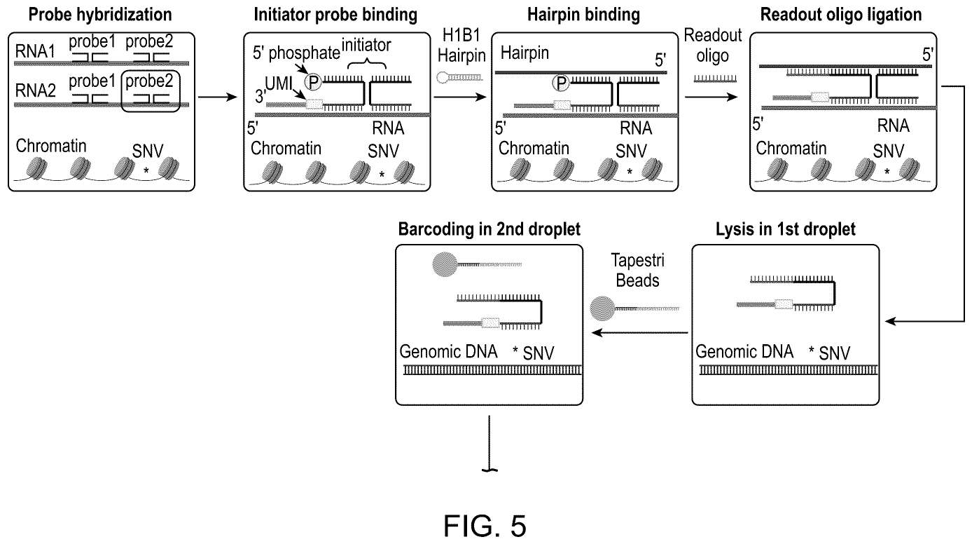

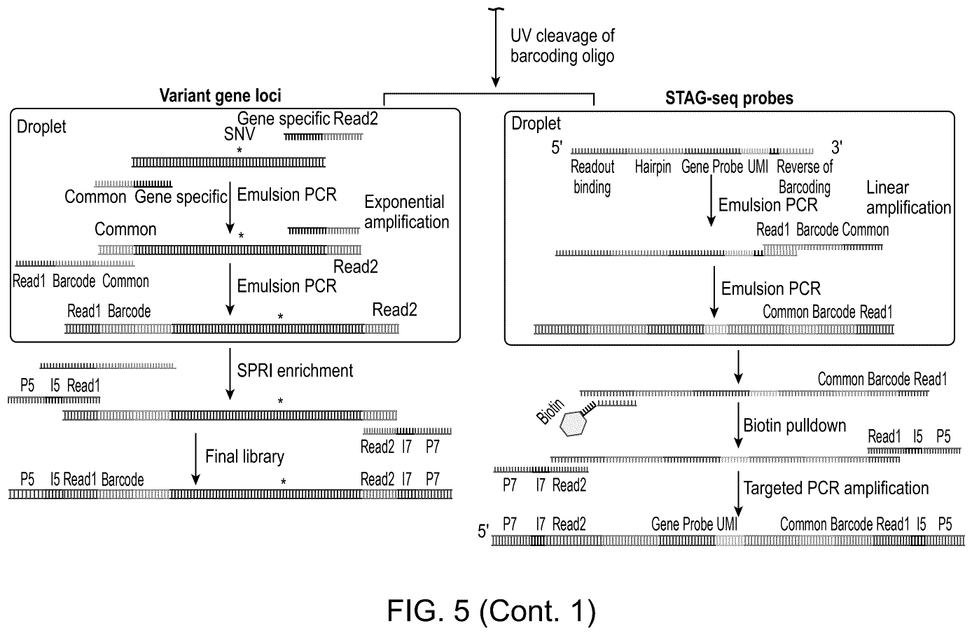

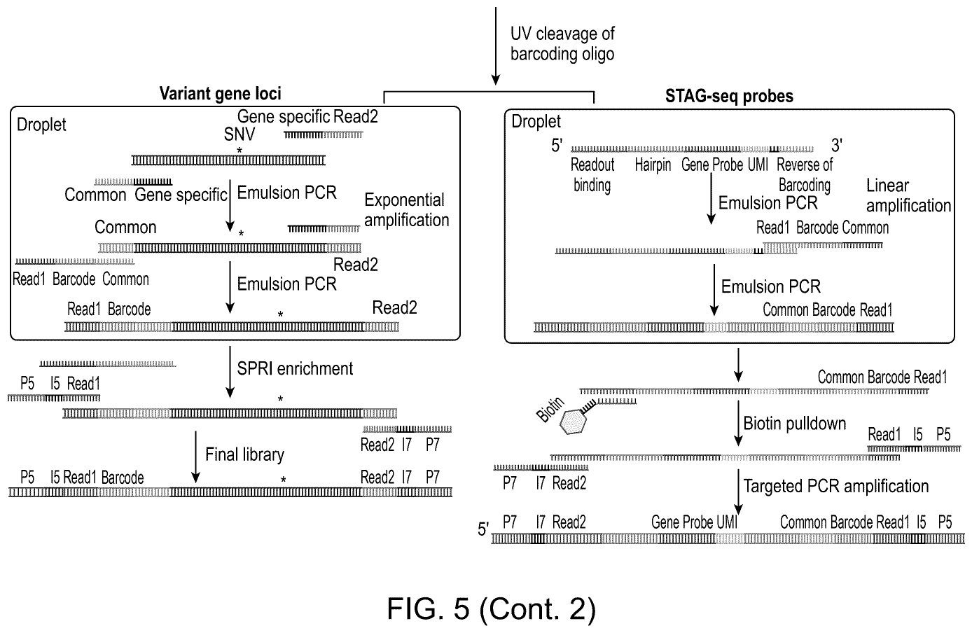

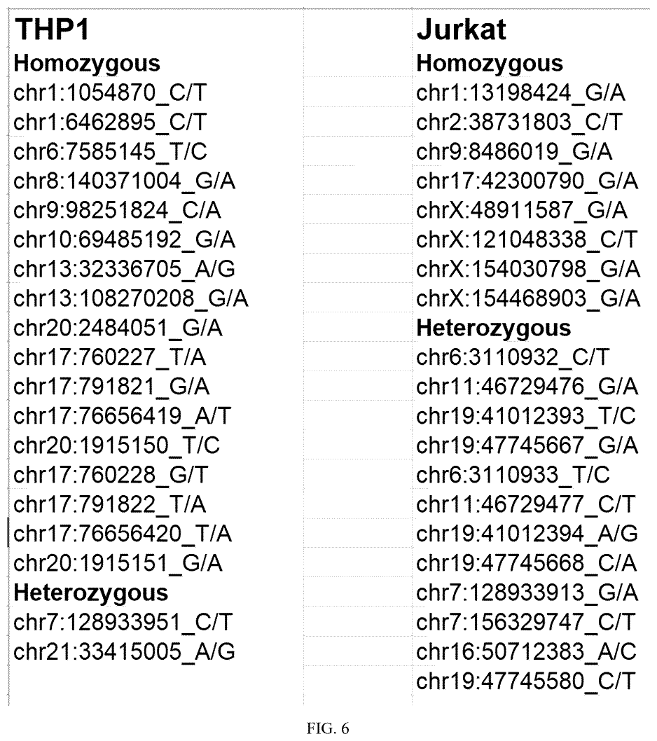

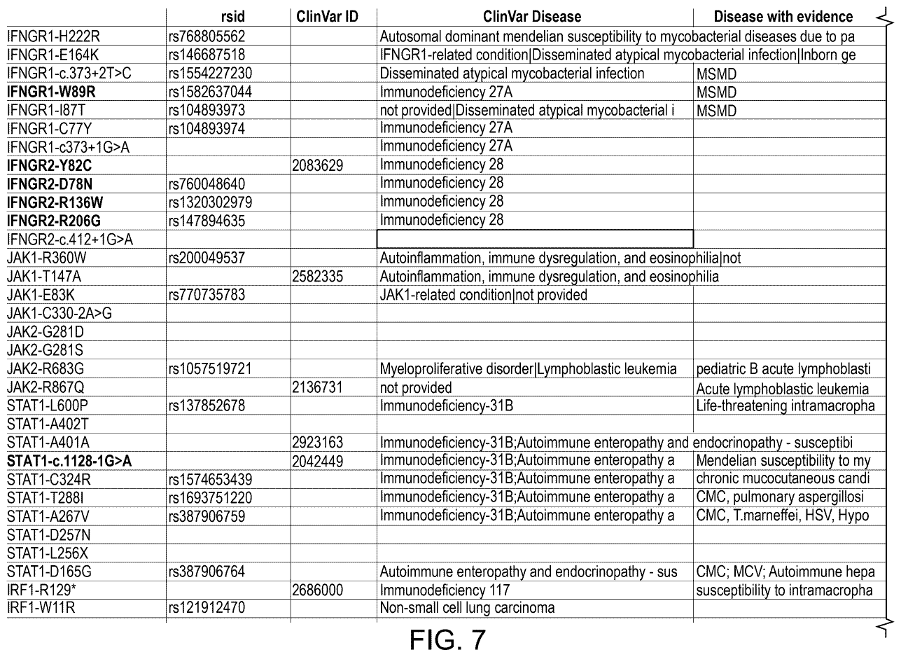

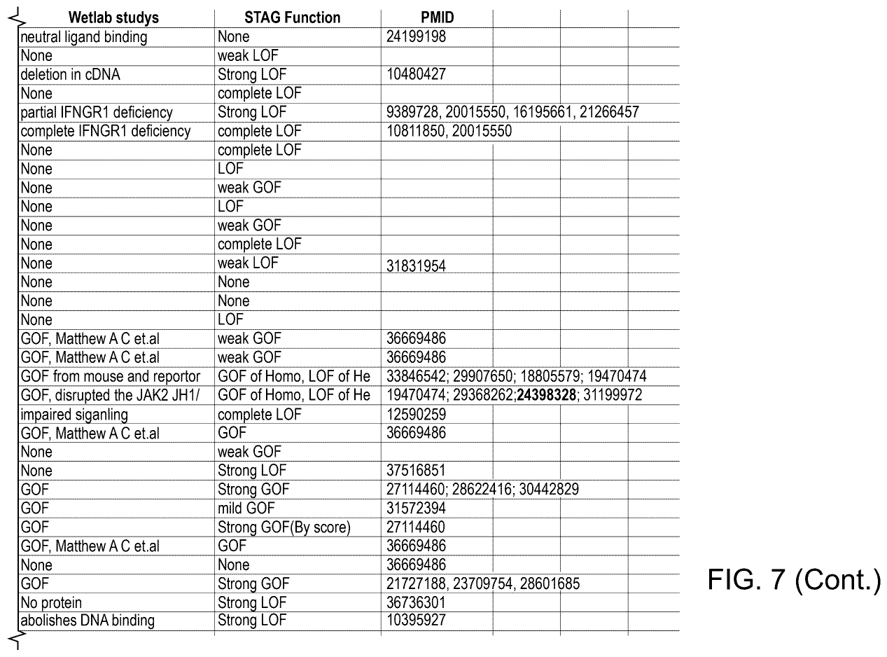

Linking genotype to phenotype in single cells is confounded by several technical challenges that require consideration. Detecting variants at the level of genomic DNA (gDNA)—whether somatic, germline inherited, or installed by genome editing—is particularly challenging in individual diploid cells, and the allele dropout rate can be high, which makes distinguishing zygosity especially difficult. Furthermore, there are no methods to allow for joint assessment of genotypes with transcriptome measurements at a scale sufficient for high-throughput genetic screens. The STAG-seq process of the instant disclosure was developed, at least in part, by adapting and integrating a droplet-based single-cell genotyping platform (Tapestri, Mission Bio) with Hybridization of Probes to RNA for sequencing (HyPR-seq) to profile gene expression (FIG. 1A and FIG. 5). HyPR-seq offers significant advantages as compared to RNAseq, as HyPR-seq enables the design of multiple probes per transcript, thus amplifying the target signal and reducing sequencing costs. Additionally, its DNA probes are compatible with the Tapestri platform's amplification process.

Cellular fixation conditions were optimized for HyPR-seq that would be compatible with single-cell gDNA recovery. Compared to live cells, it was discovered that fixation with a methanol-based approach allowed for the current process to obtain comparable detection sensitivity and coverage across multiple genomic loci. A strategy for direct capture of HyPR probes on Tapestri barcoding beads (FIG. 5) was developed. This approach was first tested using 82 probes targeting 21 transcripts in THP1 cells and efficient detection of HyPR probes (median of 37.6 UMI/probe/gene) without affecting DNA detection sensitivity was observed. Next, the optimization of the amplification of HyPR-seq probes in the STAG-seq protocol was sought. 327 HyPR probes covering 110 transcripts (plus a GFP negative control) and primers for 312 amplicons covering different genomic loci were designed and then STAG-seq was performed in human monocyte-derived macrophages (MDMs) stimulated with LPS. To balance efficiency and recovery of the HyPR and gDNA libraries, both linear and exponential amplification of the HyPR-seq probes in the emulsion were directly compared. Quantitative detection of macrophage genes (from 0.1 to 156 UMIs/probe/cell for linear amplification) and minimal background signal from GFP probes (0.02 UMIs/probe/cell for linear amplification) was observed. While both amplification strategies were sensitive and specific, linear amplification more sensitively detected inducible gene expression changes in response to LPS. Taken together, linear amplification of HyPR probes and standard exponential amplification of gDNA amplicons provided optimal quantification of dynamic gene expression programs in primary human immune cells. Having optimized the instant STAG-seq methodology, performance benchmarking was initiated. First, the single-cell fidelity of STAG-seq was assessed through a population mixing experiment in which separate samples of MDMs were hybridized with one of two probe sets targeting the same 481 genes, but with different binding sites. After hybridization, samples were mixed and subjected to STAG-seq. Out of 5,502 cells, only 89 cells were identified in which both probe sets accounted for >5% of total UMIs, representing a 1.62% collision rate, which indicates a high level of single-cell purity.

Next, establishing the scalability of transcriptome measurements was sought using STAG-seq by creating three probe pools targeting an escalating number of probes and genes: P500 (481 probes targeting 478 genes), P1000 (957 probes targeting 479 genes), and P2000 (2,010 probes targeting 1,240 genes). All the probes in P500 were also present in P1000 and P2000. Saturated sequencing revealed that sensitivity was maintained across shared probes in different sized probe pools without compromising gDNA amplicon recovery, demonstrating that STAG-seq is scalable and capable of accurately quantifying well over 1,000 genes in a single cell. In a further phase of benchmarking, STAG-seq was assessed for its capability to genotype and phenotype simultaneously in a mixed population of THP1 and Jurkat cells. A custom 2,614-probe panel targeting 1,297 genes and gDNA amplicon panel detecting 109 distinct chromosomal loci was designed. STAG-seq identified unique single nucleotide variants (SNVs) in both cell lines, separating them by genotype into different populations, and only a small proportion (2.9%) of mixed cell doublets were observed (FIG. 1). The mean variant allele frequency (VAF) was 0.92 for the homozygous SNVs and 0.53 for the heterozygous SNVs. At each locus, the mixed cells are centered around VAF 0.5 for the reference/homozygous and 0.25 for the reference/heterozygous, indicating equal capture of all alleles (FIG. 1C). To demonstrate the fidelity of concurrent genotyping and phenotyping, genotype and gene expression profiles were integrated within individual THP1 and Jurkat cells. The transcriptional states closely matched genotype identities of the different cell lines, showing a 99.6% overlap (FIG. 1D). To extend the application of STAG-seq to more complex biological samples, such as tissue analyses, scale-up was performed by designing an extensive set of 53,540 probe pairs targeting 18,081 genes (referred to as the 50K-set). To assess the genotyping quality and transcriptome fidelity in capturing cell state changes, the 50K-set was applied in conjunction with a DNA panel covering 121 genomic regions to MDMs stimulated with either lipopolysaccharide (LPS) or treated with phosphate buffered saline (PBS) as a control. Concurrently, bulk DNA sequencing was conducted to ascertain the true genotypes, alongside bulk RNA-seq and 10× 3′ scRNA-seq to profile gene expression. With STAG-seq, a median of 86.0% and 89.2% of genomic loci were confidently genotyped per cell in the PBS and LPS samples, respectively (FIG. 1E). While a small fraction of loci (11 out of 121) were genotyped in 5-50% of cells, the remaining amplicons achieved higher detection rates, resulting in a median per-locus detection rate of 99.1% and 98.4% for LPS- and PBS-treated cells, respectively (FIG. 1F). 44 heterozygous germline variants identified by bulk DNA sequencing were further evaluated. Although some loci have a higher allele dropout rate than others, an average of 91.4% genotyping accuracy was achieved, demonstrating the quality of genotyping maintained with scaled transcriptomic measurements (FIG. 1G). To determine the performance of transcript detection, the STAG-seq and 10× sample were subsampled to have an equal number of total reads (250M) and the abundance of unique molecular identifiers (UMI) in each cell was evaluated for the 18,081 genes. STAG-seq obtained a median of 9,879 and 7,603 UMI per cell for the PBS and LPS sample respectively, showing comparable detection with 10× scRNA-seq (10,435 and 7,255) (FIG. 1H). Gene expression correlation between STAG-seq and bulk RNA-seq was notably strong (Correlation Coefficients=0.86 (FIG. 1I), comparable to that observed between 10× and bulk RNA-seq (Correlation Coefficients=0.82 (FIG. 1J). A pseudobulk differential expression analysis further demonstrated that STAG-seq accurately reflected macrophage state changes in response to LPS stimulation with a correlation coefficient of 0.87 (FIG. 1K). Overall, these findings underscore the robustness and sensitivity of STAG-seq in concurrently detecting single-cell DNA and RNA, positioning this technology as a powerful tool for phenotyping genetic variants at scale.

### Example 3: STAG-Seq Enabled Phenotyping Variants at Scale with Genotype by Stimulation Matrix Screens in Innate Immunity

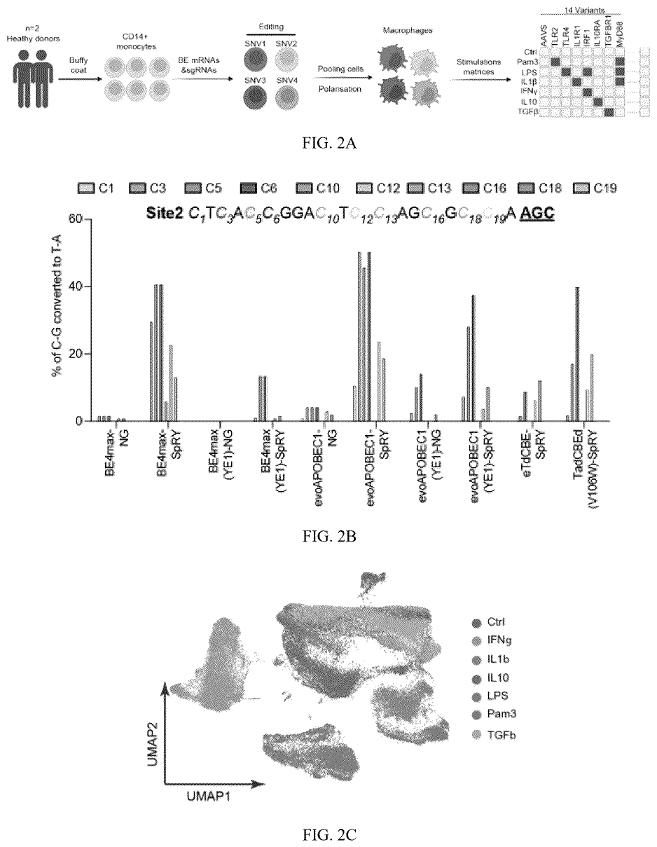

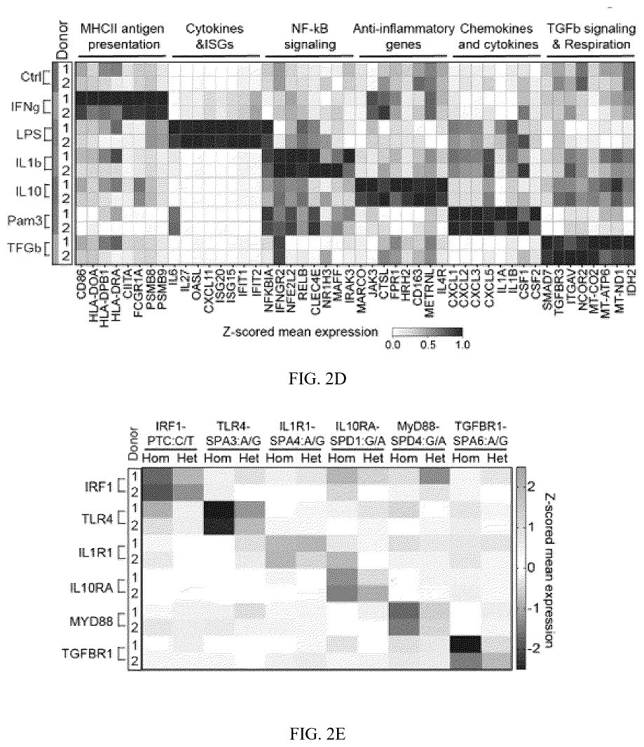

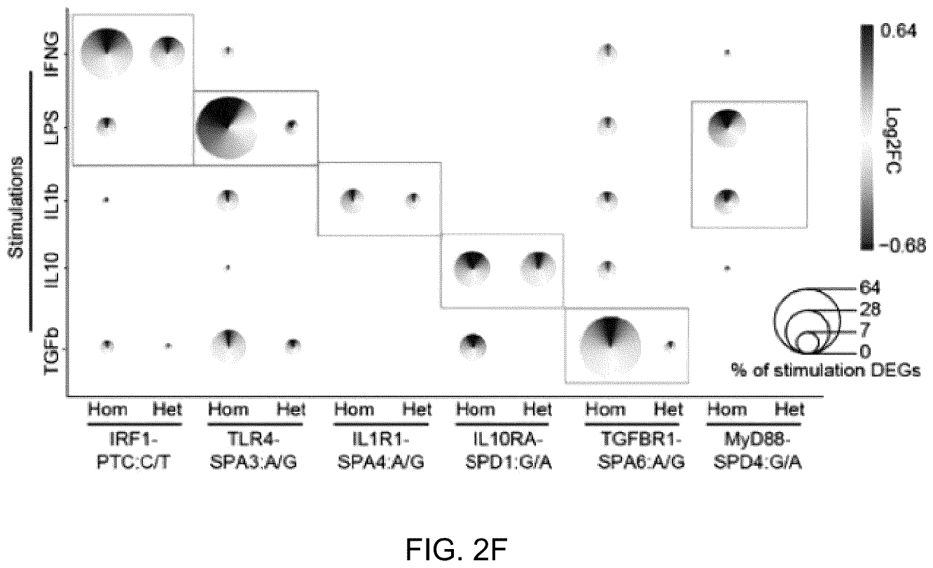

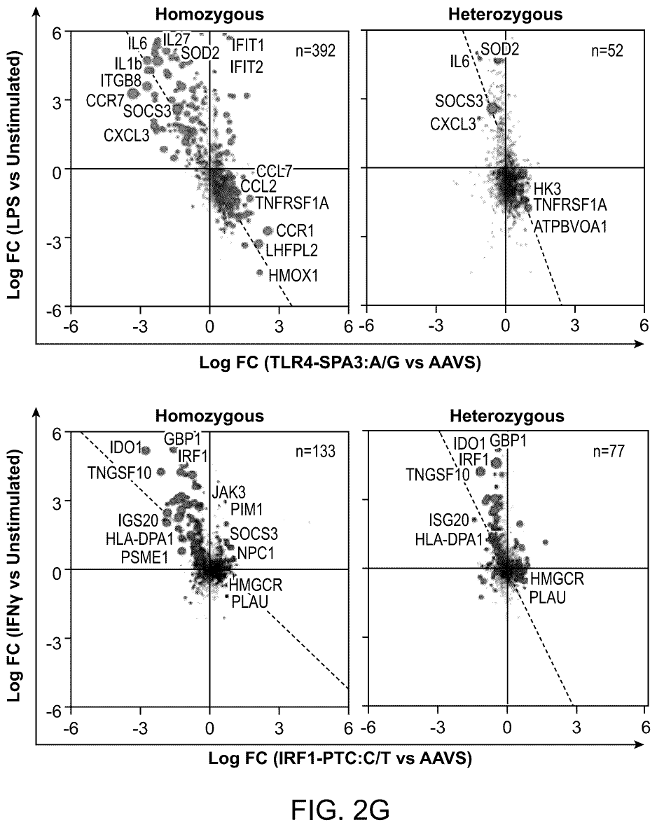

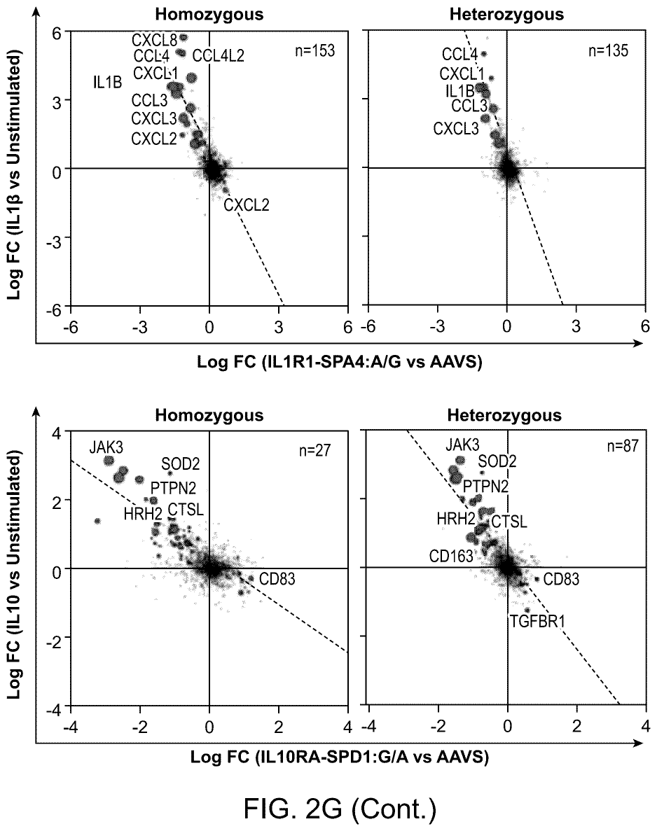

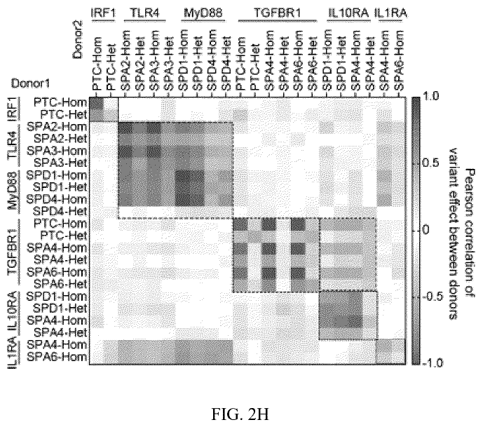

Innate immune cells, such as macrophages and dendritic cells, express a diverse array of pattern recognition receptors and cytokine receptors, enabling them to respond to specific pathogen threats. Variants associated with innate immunodeficiencies may manifest phenotypes only in particular cell types and under specific biological contexts. STAG-seq was developed with the throughput to support the systematic study of variants in a matrix format wherein multiple variants are individually installed in primary immune cells by base editors and phenotyped across a panel of extrinsic stimuli. Focusing on a panel of innate immune stimuli, key genes in pathways downstream of these signals were targeted in MDMs and the ability of STAG-seq to reveal both monoallelic and biallelic variant effects was evaluated. First, base editing was optimized in MDMs through electroporation of synthetic sgRNA targeting loci of interest and mRNA encoding various base editors. To expand the range of targetable variants and enhance editing efficiency and precision, a toolbox was assembled to produce base editor mRNAs with combinations of different deaminase variants (evoAPOBEC1, BE4Max, ABE8e and TadCBE) with PAM-flexible SpCas9 variants (NG and SpRY), as well as to introduce the narrow-window mutations into the deaminases (Kim et al. Nat. Biotechnol., 35: 371-376; Zhou et al. Nature, 571: 275-278). By targeting sites with NGG or NRY PAMs, evoAPOBEC1(YE1)-SpRY, TadCBEd(V106W)-SpRY and ABE8e(F148A)-SpRY were found to achieve high efficiencies (FIG. 2B). Next, a “matrix screen” was conducted in MDMs isolated from two donors, the cells were treated with various stimuli (PBS, LPS, IFN-γ, Pam3CSK4, IL1β, IL10 and TGFβ) and 2,740 probes targeting 1,275 genes involved in key immune pathways were applied as transcriptomic readouts. Using base editors, 14 nonsense or splice site mutations in receptors and key signaling proteins downstream of these stimuli (TLR2, TLR4, MyD88, IRF1, IL1R1, IL10RA, and TGFBR1) were introduced, along with on-target control edits in the safe harbor AAVS1 locus. Pooling all variant cells for each stimulation allowed for examination of the specific responses of variant-stimulation pairs (FIG. 2A). The stimulations induced distinct gene expression programs that were shared by both donors (FIG. 2C). Furthermore, STAG-seq discerned the unique response signatures for each stimulus: IFN-γ upregulated antigen presentation-related genes; LPS and Pam3CSK4 predominantly induced proinflammatory cytokine production; IL1β triggered NF-kB signaling; IL-10 specifically upregulated anti-inflammatory genes; and TGFβ primarily impacted TGFβ signaling and cell metabolism characteristic of alternative macrophage activation (FIG. 2D). To assess the functional impact of edited variants, effects on expression of their respective target genes were first examined. Homozygous mutations generally reduced target gene expression, while heterozygous mutations showed subtler but significant decreases, demonstrating the ability of STAG-seq to detect and distinguish cis effects of homozygous and heterozygous variants (FIG. 2E). This is also consistent with the fact that nonsense or splice-site mutations usually trigger the nonsense-mediated mRNA decay (NMD) pathway to destabilize the mutated mRNA. Trans-regulatory effects were then investigated by measuring the proportion of stimulation-responsive genes affected by each variant. Homozygous receptor variants (TLR4, IL1R1, IL10RA, and TGFBR1) dramatically altered responses to their respective ligands (LPS, IL-1β, IL-10, and TGFβ) with minimal effects from other stimuli, while heterozygous variants exhibited similar patterns but with reduced magnitude (FIG. 2F). Variants with pleiotropic effects were also identified. For example, the IRF1 homozygous variant strongly impacted the IFN-γ signaling response and weakly but robustly impacted the LPS response (40.6% of IFN-γ signaling, 5.6% of LPS). Similarly, MyD88 variants altered both LPS and IL-1β responses (22.8% of LPS, 9.7% of IL1β) (FIG. 2F). These results aligned with previous reports that IRF1 acts as a pioneer transcription factor mediating signaling from LPS and IFN-γ, while MyD88 serves as an essential adapter for both TLRs and IL-1β receptors (Muzio et al. Science, 278: 1612-1615; Medzhitov et al. Mol. Cell, 2: 253-258; Song et al. Cell Rep., 34: 108891; De Benedetti et al. Nat. Rev. Rheumatol., 17: 678-691). To further characterize how edited variants influence stimulation-induced gene expression, specific gene sets were analyzed. Homozygous TLR4 variants corresponding to nonsense mutations or splice site disruption dramatically reduced LPS-induced cytokines and chemokines (IL6, IL27, IL1β, and CXCL3) while increasing LPS-suppressed genes (CCRI, HMOX1, LHFPL2, TNFRSF1, and CLEC7A), indicating a strong suppression of LPS responses in variant cells (FIG. 2G). The corresponding heterozygous variants showed partial blockade with weaker reduction of IL6 and CXCL3 expression (FIG. 2G). Similarly, homozygous variants in IRF1, ILIR1, and IL10RA strongly inhibited gene expression responses to IFNγ, IL-10, IL-10, and TGFβ treatment, respectively, with heterozygous variants exhibiting the same trends but with smaller magnitude changes and fewer differentially expressed genes (DEGs) (FIG. 2G). Importantly, the effects of homozygous and heterozygous variants were consistent across different variants of the same gene, different genes within the same pathway (e.g., TLR4 and MyD88), and the same gene in two independent donors (FIG. 2H). Taken together, STAG-seq enabled the systematic identification of mono- and biallelic variant functions in primary human immune cells under diverse biological stimuli, providing a powerful platform for high-throughput matrix screens in functional genomics.

### Example 4: STAG-Seq Discerned Function for Clinically Relevant Coding Variants Impacting Core Immune Pathways

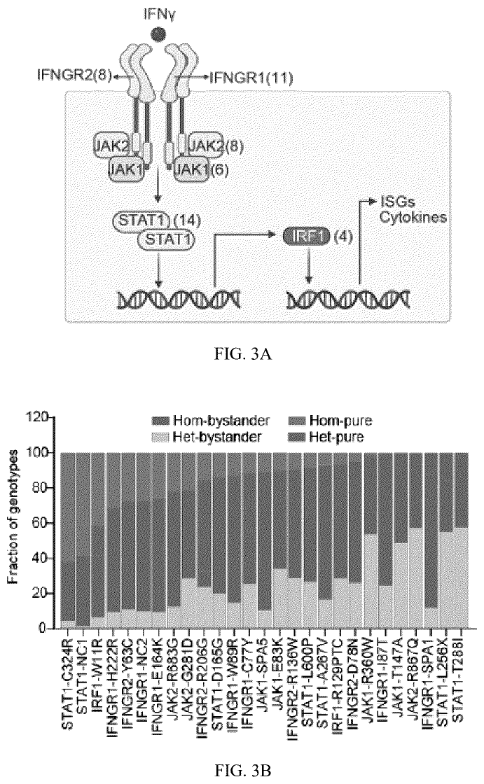

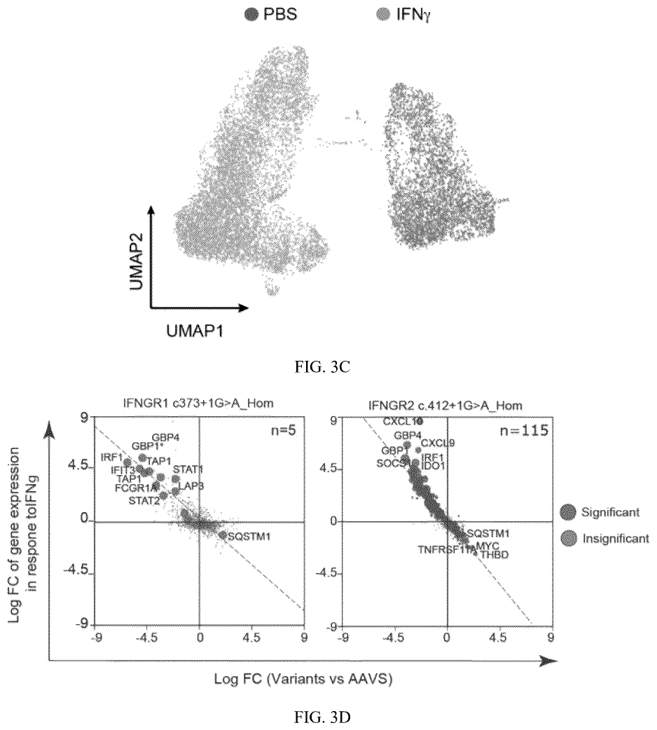

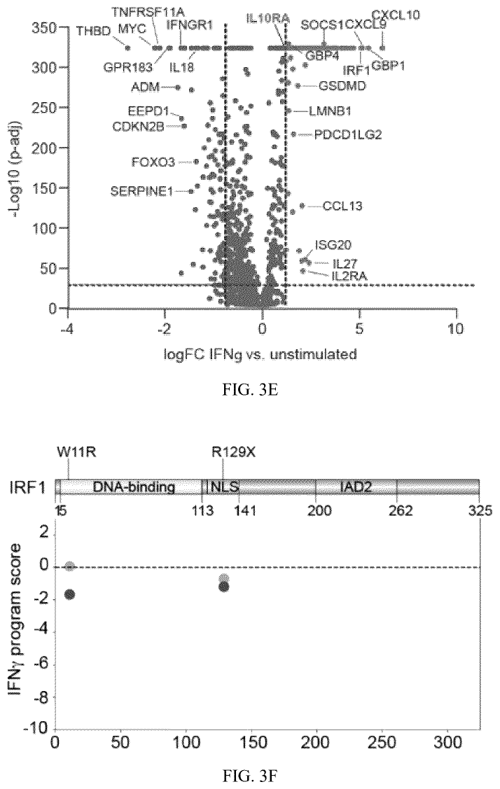

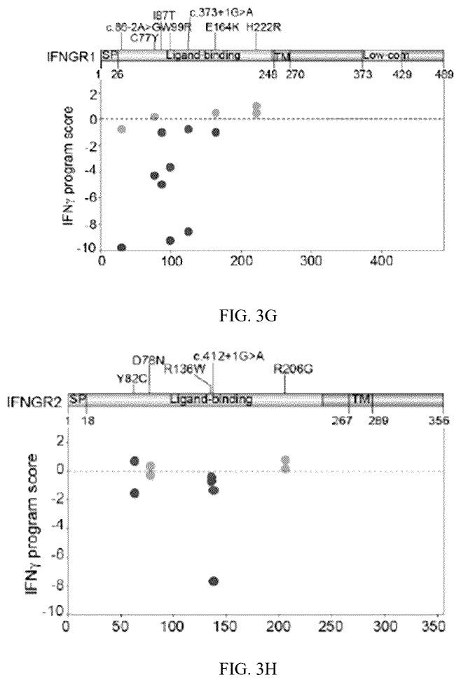

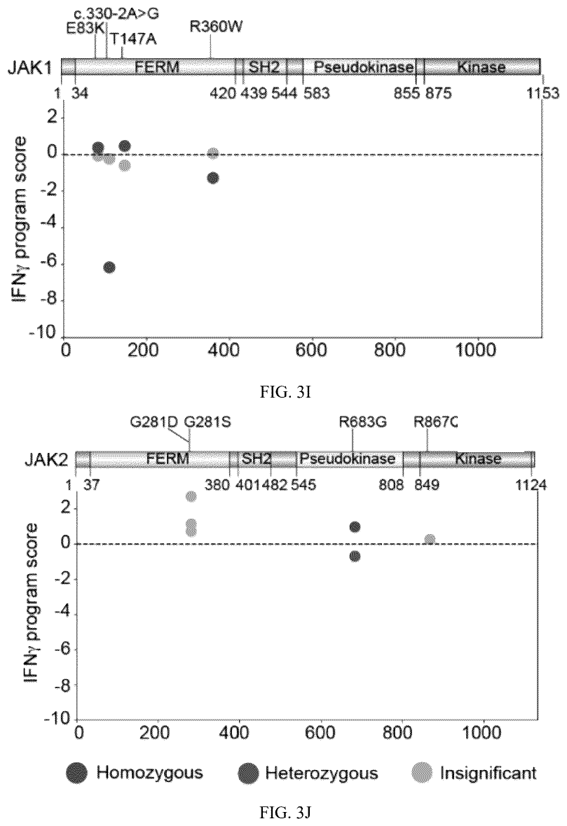

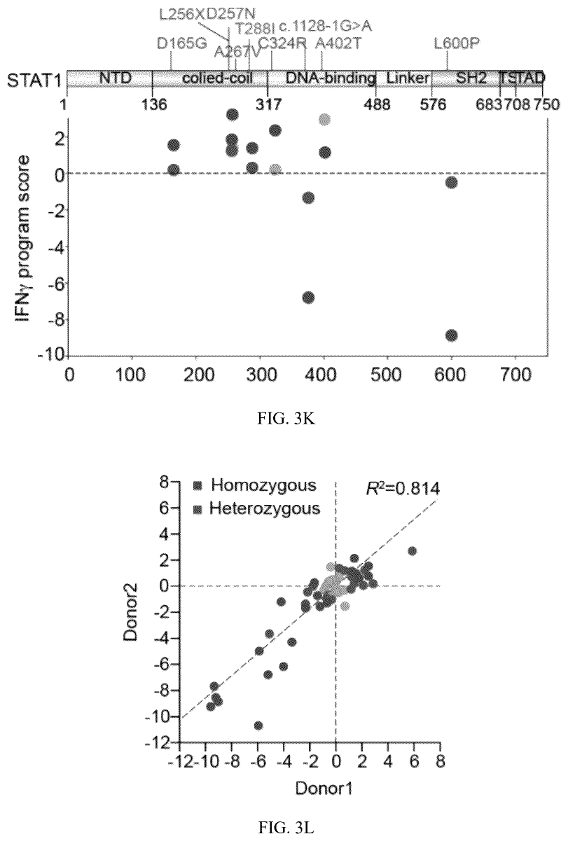

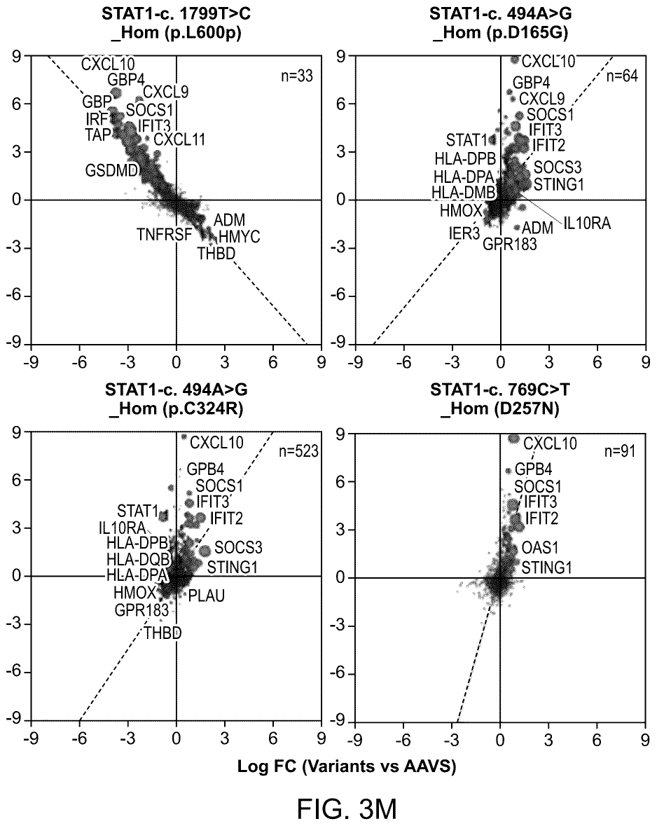

Deployment of STAG-seq to reveal the functions of clinically relevant coding variants was initiated, aiming to accelerate mechanistic understanding of genetic variants of uncertain significance (VUS). Hundreds of variants have been identified to date that impact different components of the interferon gamma (IFN-γ) signaling pathway through distinct mechanisms of action. Given its central role in autoimmunity, infectious disease, and cancer, variants associated with a variety of disease phenotypes that affect key IFN-γ pathway components—including IFNGR1, IFNGR2, JAK1, JAK2, STAT1, and IRF1—were selected from OMIM and ClinVar (FIG. 3A). 24 Clinvar and 6 control variants were further prioritized across the 6 genes, achieving ≥10% editing efficiency in MDMs isolated from 2 donors for profiling with STAG-seq. Overall, editing efficiencies varied by site, as did bystander editing rates, which ranged from 12% to 88% at different loci, supporting the necessity of precise genotyping to ascertain true variant function (FIG. 3B). After editing, all the cells were then pooled together and treated with PBS or IFN-7. With unsupervised clustering by the expression of 1,275 genes (2,740 probes), IFN-γ stimulated cells largely separated from PBS treated cells (FIG. 3C). Notably, two positive control variants at the splice sites of IFNGR1 and IFNGR2 nearly abolished cellular response to IFN-γ (FIG. 3D). Even with only five homozygous IFNGR1 control variant (c.88-2A>G) cells, STAG-seq detected statistically significant inhibition of IFN-γ pathway genes (FIG. 3D). To directly compare the phenotypes and effect sizes of genetic variants, IFN-γ response scores were derived that summarize the transcriptional responses to IFN-γ for each “pure” homozygous and heterozygous variant lacking bystander mutations. Accordingly, negative scores indicated impaired IFN-γ response, whereas positive scores reflected enhanced signaling (FIGS. 3E-3K). With this approach, individual variant phenotypes were remarkably consistent across donors, underscoring the robustness of STAG-seq for functional profiling (FIG. 3L). Importantly, the variant effects aligned with the clinical reports. For instance, the immunodeficiency-associated STAT1 variant rs137852678 (L600P) exhibited complete inhibition of IFN-γ signaling in homozygous cells, aligned with its clinical classification as autosomal recessive complete deficiency (FIGS. 3K and 3M; Boisson-Dupuis et al. Curr. Opin. Immunol., 24: 364-378). Having established a framework for accurately genotyping edited variants, molecular and clinical phenotypes were integrated. Variants of IRF1, IFNGR1, IFNGR2, JAK1, and STAT1 that are associated with immunodeficiencies and susceptibility to viral and mycobacterial infections exhibited loss-of-function (LOF) phenotypes and reduced IFN-γ response scores (FIGS. 3F-3K). In addition to variants linked to infectious disease, cancer-associated variants in the IFN pathway, including JAK2 variant rs1057519721 (pR683G), which has been implicated in pediatric acute lymphoblastic leukemia (ALL; Mullighan et al. Proc. Natl. Acad. Sci. U.S.A, 106: 9414-9418; Shan et al. Nat. Struct. Mol. Biol., 21: 579-584; Downes et al. Front. Cell Dev. Biol., 10: 942053; Hammarén et al. J. Allergy Clin. Immunol., 143: 1549-1559.e6) were also characterized. STAG-seq demonstrated an allele-dose effect for this variant, where homozygous cells exhibited a gain-of-function (GOF) phenotype, whereas heterozygous cells displayed a LOF phenotype, (FIG. 3J). STAT1 variants exhibited the most diverse range of disease associations and functional effects based on IFN-γ responses (FIG. 3K). As a well-known nonredundant signal transducer and transcription activator of types I, II, and III IFN as well as IL-27 (O'Shea et al. N. Engl. J. Med., 368: 161-170) pathways, STAT1 mutations underlie diverse clinical phenotypes. The variants classified as complete LOF by STAG-seq are associated with severe immunodeficiencies and susceptibility to viral and mycobacterial infections (Toubiana et al. Blood, 127: 3154-3164; Dupuis et al. Nat. Genet., 33: 388-391). In striking contrast, variants classified as GOF based on IFN-γ response scores are associated with autoimmunity, and paradoxically, susceptibility to fungal infection (Toubiana et al. Blood, 127: 3154-3164; Okada et al. J. Clin. Immunol., 40: 1065-1081). These findings are consistent with recent work showing increased Th1 responses underlying impaired antifungal immunity (Break et al. Science, 371: eaay5731). STAG-seq identified and corroborated additional GOF STAT1 variants associated with autoimmunity and fungal infection, including rs1574653439 (pC324R), rs1693751220 (pT288I), rs387906759 (pA267V), rs387906764 (pD165G), and also one variant (pD257N) that was identified in a CRISPR screen (FIGS. 3K and 3M; Toubiana et al. Blood, 127: 3154-3164; Coelho et al. Cancer Cell, 41: 288-303.e6). Notably, although these GOF variants enhanced the expression of most IFN-γ-induced genes (CXCL10, GBP4, SOCS1, IFIT2, SOCS3 and STING1), some of them inhibited STAT 1 and MHC-II expression after IFN-γ stimulation, indicating a more complex phenotype (FIG. 3M). Additionally, different GOF variants exhibited distinct transcriptional profiles at steady state and after stimulation. While IFN-γ stimulation potentiated the expression of IL10RA in control (AAVS1 edited) cells, this response was attenuated by rs1574653439 (pC324R) and enhanced by rs387906764 (pD165G) (FIG. 3E), suggesting distinct mechanisms of action between GOF variants. Together, these findings demonstrated STAG-seq's ability to systematically resolve both LOF and GOF phenotypes across a spectrum of effect sizes, linking genetic variation to immune dysfunction and disease risk. Finally, to further validate our findings, STAG-seq-discovered phenotypes were compared with ClinVar classifications. All 14 ClinVar-classified pathogenic or likely pathogenic variants exhibited significant IFN-γ response scores, consistent with their known disease associations. Notably, 5 out of 10 variants of uncertain significance (VUS) also displayed significant IFN-γ response scores, indicating that these variants may indeed affect gene function. These results highlighted STAG-seq's potential for reclassifying VUS, offering a powerful framework to bridge genetic variation with functional consequences at single-cell resolution.

### Example 5: STAG-Seq Enabled Functional Dissection of Noncoding Variants within a Complex Locus Linked to Autoimmune Disease

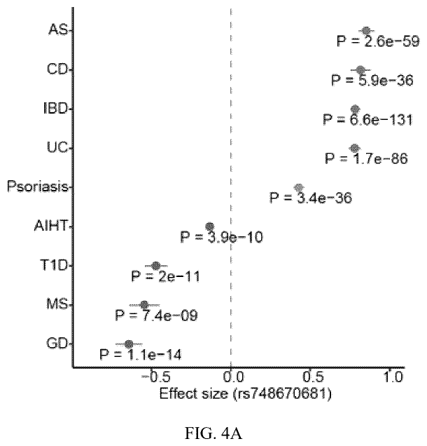

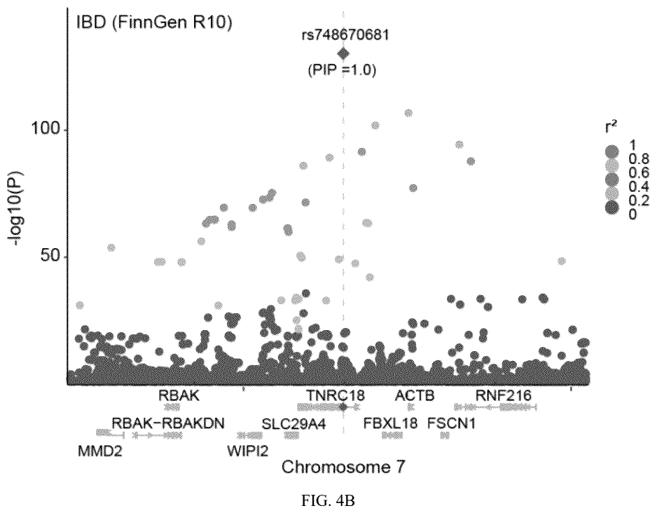

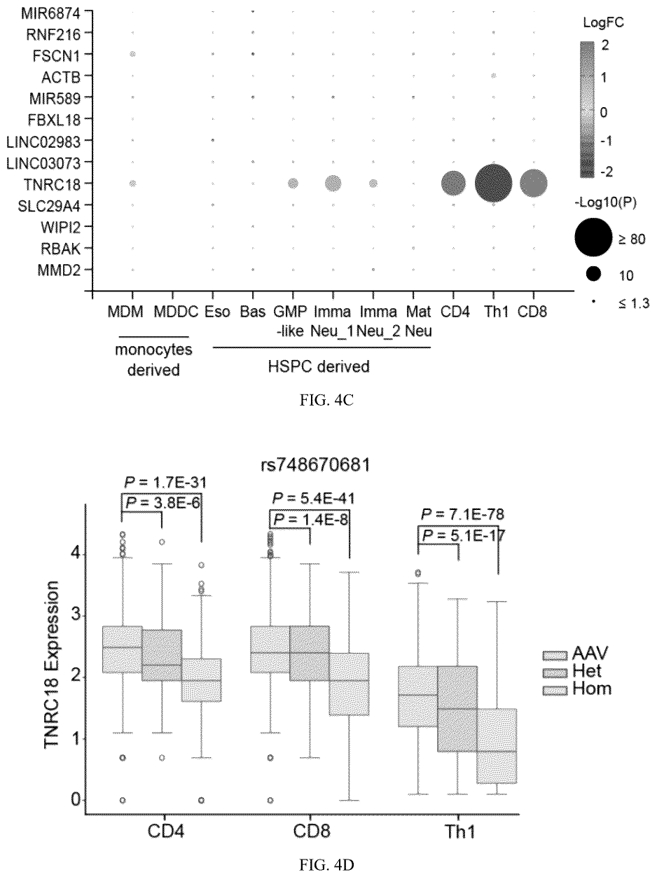

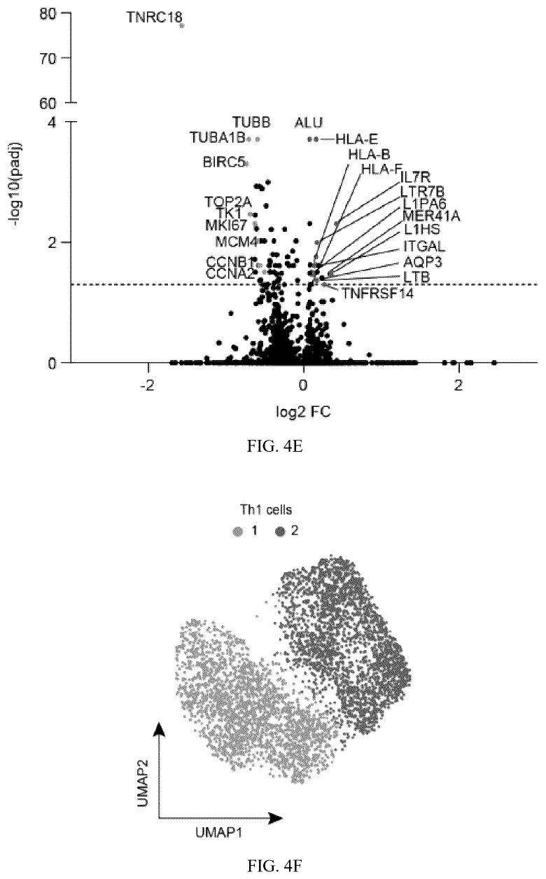

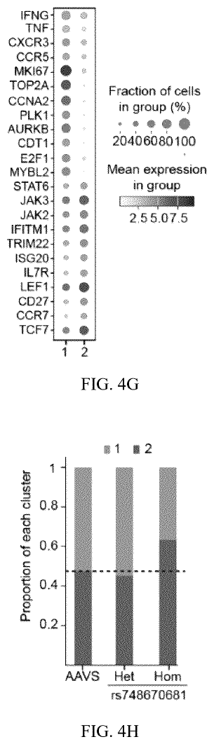

Population genetics and GWAS predominantly identify noncoding variants with moderate effect sizes on gene expression, and mapping noncoding variants to functional effects on neighboring genes is complicated by cell type specificity and availability of samples with desired variants. This is the case for rs748670681 (chr7:5397122 C/T), a Finnish-enriched noncoding variant on chromosome 7 that exhibits a unique profile of disease associations (MAF=3.6% in FinnGen; 114-fold Finnish enrichment compared to non-Finnish Europeans). The minor allele imparts risk for IBD, ankylosing spondylitis, and psoriasis (OR=2.2, 2.3, and 1.5, respectively); but is protective for T1D, multiple sclerosis, Grave's disease, and autoimmune hypothyroidism (OR=0.6, 0.6, 0.5, and 0.9, respectively; FIG. 4A). The locus (800 kb) harboring rs748670681 contains several protein-coding genes (FIG. 4B). Although the variant resides within an intron of TNRC18, any gene in the locus could be a cis eQTL target, necessitating empirical evaluation using STAG-seq. Given its strong autoimmune association, the variant was introduced into human immune cells of both myeloid and lymphoid lineages. These include monocyte derived macrophages or dendritic cells (MDDCs), granulocytes differentiated from human hematopoietic stem and progenitor cells (HSPCs), activated CD4 and CD8 T cells, as well as ex vivo differentiated Th1 cells. Comparing the variant cells to the AAVS1 edited cells, cis effects of rs748670681 were first examined on all genes within the locus and across different immune cell types. The homozygous variant significantly downregulated TNRC18 expression in specific cell types without impacting other genes (FIG. 4C). A mild cis eQTL effect was observed in granulocyte-macrophage progenitor (GMP)-like cells and immature neutrophils, but not in other myeloid cell types (FIG. 4C). Notably, the cis-eQTL effect was most pronounced in T cells, particularly in Th1 cells, where homozygous variant cells exhibited stronger downregulation of TNRC18 than heterozygous cells, confirming both monoallelic and biallelic cis-eQTL effects (FIG. 4D). TNRC18 has recently been identified as an epigenetic reader of H3K9me3, enforcing silencing of endogenous retrotransposons in multiple cancer cell lines, although its functions in primary immune cells are still unclear (Zhao et al. Nature, 623: 633-642). To examine potential trans effects of rs748670681, 1,234 probes targeting 224 retrotransposons we designed at family or subfamily levels and 4,605 probes targeting 2,021 coding genes were also designed. Among all tested cell types, rs748670681 showed the strongest cis effect on TNRC18 expression in Th1 cells, with broad reactivation of ERVs, human-specific L1HS (LINE-1 Class) and the Alu element (SINE class) (FIG. 4E). The variant cells also upregulated expression of IL7R, MHCI molecules and TNF superfamily members (TNFRSF14, LTB), while downregulating cell cycle genes, which indicated a shift from proliferation to a survival/memory-like cell state (FIG. 4E; Colpitts et al. J. Immunol., 182: 5702-5711; Li et al. J. Exp. Med., 198: 1807-1815; Kondrack et al. J. Exp. Med., 198: 1797-1806; Glatigny et al. Sci. Rep., 5: 7834; Hara-Chikuma et al. J. Exp. Med., 209: 1743-1752). To further evaluate the impact of rs748670681 on cell state, the heterogeneity of Th1 cells was investigated, with the discovery that Th1 cells segregated into two major clusters (FIG. 4F). Cluster 1 cells expressed Th1 cytokines and chemokines (IFNG, TNF, CXCR3) and show a clear feature of cell cycle progression and mitosis, suggesting they are activated and proliferating cells (FIG. 4G). Cluster 2 cells expressed higher levels of stemness/memory genes (IL7R, TCF7, LEF1, CD27) and tissue homing markers (CCR7 CXCR6), suggesting that they are memory-like cells that transiently enter into quiescent state for long-term survival (FIG. 4G). Those cells also exhibit induction of ISGs (FIG. 4G). Given the fact that Th1 cells are a major source of IFN-γ and respond to it through positive feedback, cluster 2 cells might be primed for inflammatory responses. This is also consistent with a previous report that TEs contribute to the expression of ISGs by facilitating the binding of IFN-γ induced transcription factors (Chuong et al. Science, 351: 1083-1087). Compared to the AAVS edited control, rs748670681 homozygous variant leads to a significantly increased proportion of memory-like cells (Cluster 2, X²=44.14, p<0.0001), indicating a variant-driven shift toward long-live memory states, which may contribute to tissue inflammation (FIG. 4H). Together, these findings revealed a cell type specific cis- and trans-effect of the autoimmunity associated variant rs748670681, and also provide mechanistic insights into its potential role in autoimmune disease. Thus, by integrating genome editing, single-cell genotyping and transcriptomic profiling across diverse immune cell states, STAG-seq uncovered a functional mechanism for a rare noncoding variant in primary immune cells, providing a powerful framework for dissecting the molecular basis of autoimmune risk variants.

Understanding how genetic variation—germline or somatic—contributes to disease remains a central challenge in human genetics. While genome-wide association studies (GWAS) and clinical sequencing have identified thousands of disease-associated variants, the vast majority remain functionally uncharacterized, particularly those with subtle regulatory effects or that function in a context-dependent manner. Furthermore, the heterozygous nature of most variants in the human genome underscores the critical need to resolve allele-specific contributions to disease phenotypes. To address those challenges, STAG-seq, a droplet-based method for single-cell gDNA amplicon sequencing combined with targeted transcriptomics, has been developed and disclosed herein. STAG-seq enables parallel genotyping of hundreds of loci and profiling of thousands to tens of thousands of genes across 10⁴-10⁵ cells in a single experiment. This approach offers three key advancements: 1) high-sensitivity genotyping with minimal allelic dropout (≤2.5%), enabling precise detection of both homozygous and heterozygous variants at scale; 2) enhanced RNA detection with better quantitative accuracy than standard 3′ transcriptome scRNA-seq, empowering functional studies of variants with subtle effect sizes; and 3) flexible targeting of genomic regions and RNA transcripts, allowing researchers to focus on specific biological questions while reducing sequencing costs. Additionally, the in-house protocol disclosed herein for synthesizing customized probe pools simplifies adoption of this technology. The sensitivity of STAG-seq arises from its direct, accurate gDNA sequencing with exceptionally low allelic dropout rates, enabling experiments that are infeasible with existing methods. By comparison, Perturb-seq approaches rely on detection of sgRNA in edited cells based on the prior that cells expressing these guides will have a predictable editing outcome (Martin-Rufino et al. Cell, 186: 2456-2474.e24; Morris et al. Science, 380: eadh7699). However, editing efficiencies vary by locus (Li et al. Cell, 187: 2411-2427.e25), and bystander editing is common. Proxy-based genotyping approaches utilize a transcribable decoy genomic target sequence to detect edits and infer editing at the endogenous genomic locus (Ryu et al. Nat. Genet., 56: 925-937; Sanchez-Rivera et al. Nat. Biotechnol., 40: 862-873; Kim et al. Nat. Biotechnol., 40: 874-884). This genotype inference is an improvement but fails to directly reveal variant zygosity in editing endogenous loci and stochastic bystander editing. Alternatively, direct genotyping from the transcriptome or chromatin fragments enables detection of potential edits, but is limited by cell dropouts, access to the region of interest, and gene expression levels (Izzo et al. Nature, 629: 1149-1157; Olsen et al. Nat. Methods, 22: 477-487; Nam et al. Nature, 571: 355-360). Paired genome and transcriptome sequencing approaches represent an advancement towards improved genotyping and coupling to single cell gene expression profiles (Rodriguez-Meira et al. Mol. Cell, 73: 1292-1305.e8; Macaulay et al. Nat. Methods, 12: 519-522; Macaulay et al. Nat. Protoc., 11: 2081-2103; Dey et al. Nat. Biotechnol., 33: 285-289; Yu et al. Sci. Adv., 9: eabp8901; Olsen et al. bioRxiv, 2023: doi:10.1101/2023.02.09.527940; Baglaenko et al. bioRxiv, 2024: doi:10.1101/2024.03.28.587175), although these are largely constrained to the throughput of arrayed plate-based formats. In contrast, STAG-seq offers orders of magnitude higher throughput with added sensitivity for paired genotyping and gene expression profiling, which could further be used to validate and improve AI models for genomics (Avsec et al. Nat. Methods, 18: 1196-1203; Linder et al. Nat. Genet., 2025: doi:10.1038/s41588-024-02053-6; Pampari et al. bioRxiv, 2025: doi:10.1101/2024.12.25.6302210). By integrating base editing with single-cell multi-omics, STAG-seq addresses a significant obstacle in translating discoveries from human genetics into mechanistic insights of disease. STAG-seq's ability to accurately genotype installed edits while sensitively capturing transcriptomic changes in the same single cell unlocks systematic exploration of genotype-phenotype relationships across diverse disease contexts.

STAG-seq is uniquely powered to functionally characterize variants with a broad range of effect sizes. This is largely due to accurate and direct sequencing of gDNA with a low allele dropout rate, which allows for experiments that are intractable for other methods. By comparison, indirect genotype inference in Perturb-seq approaches rely on detection of sgRNA in edited cells based on the prior that cells expressing these guides will have a predictable editing outcome. However, editing efficiencies vary by locus, and bystander editing is common. Proxy-based genotyping approaches utilize a transcribable decoy genomic target sequence to detect edits and infer editing at the endogenous genomic locus. This genotype inference can improve accuracy, but is still an estimate that is susceptible to discrepancies in editing endogenous native loci and stochastic bystander editing. Alternatively, direct genotyping from the transcriptome enriches for detection of potential edits, but is limited to coding regions and genes that are expressed above the limit of detection. Paired genome and transcriptome sequencing approaches represent an advancement towards accurately genotyping edits and coupling them to single cell gene expression profiles, although these are largely constrained to the throughput of arrayed plate-based formats. In contrast, STAG-seq offers orders of magnitude higher throughput with added sensitivity for paired genotyping and gene expression profiling. STAG-seq allows for the examination of up to 15K cells in a single reaction, with a cell recovery rate of approximately 10%. A notable advancement is in genotyping accuracy. STAG-seq achieves an average allelic dropout rate of 2.5%, which is a significant improvement over conventional genotyping methods (e.g., genotyping of targeted loci (GoT) or genotyping of targeted loci with single-cell chromatin accessibility (GoT-ChA)).

The development of STAG-seq addresses a significant obstacle in translating discoveries from human genetics into mechanisms of disease. In conjunction with base editing in primary human cells, STAG-seq enables highly accurate genotyping of installed edits and remarkable sensitivity of gene expression within the same single cell. A broad range of applications for STAG-seq is envisioned in establishing genotype-phenotype relationships that assign mechanistic function to germline variants and somatic mutations associated with a variety of human diseases. Ultimately, new approaches for deciphering the genetic basis of human disease—and associated molecular mechanisms—hold great potential for guiding the development of new therapeutic strategies.

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize or be able to ascertain using no more than routine experimentation, many equivalents to the specific embodiments of the disclosure described herein. Such equivalents are intended to be encompassed by the following claims.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for generating paired barcoded gene-specific amplicons and barcoded RNA expression tracking constructs in a population of individual discrete volumes each corresponding to a single cell of a population of cells, the method comprising:
(i) contacting a population of cells with a first selection of oligonucleotides capable of detecting target RNA levels and selectively generating an RNA expression tracking construct in the presence of the target RNA;
(ii) encapsulating individual cells of the population of cells in first individual discrete volumes, said first individual discrete volumes comprising cell lysis buffer and proteases, thereby generating lysed individual cells in the first individual discrete volumes;
(iii) contacting the lysed individual cells in the first individual discrete volumes with a barcoding mixture, said barcoding mixture comprising bead-attached barcoding oligonucleotides, and genotyping probes under conditions sufficient to generate a population of lysed individual cells and individual beads comprising bead-attached barcoding oligonucleotides and genotyping probes in the second individual discrete volumes;
(iv) incubating the population of lysed individual cells and individual beads comprising bead-attached barcoding oligonucleotides and genotyping probes in the second individual discrete volumes under conditions sufficient to allow for:
amplification of the genotyping probes to proceed, thereby generating gene-specific amplicons in the second individual discrete volumes; and
polymerase-mediated extension of the bead-attached barcoding oligonucleotides along the gene-specific amplicons and along the RNA expression tracking constructs in the second individual discrete volumes, thereby generating barcoded gene-specific amplicons and barcoded RNA expression tracking constructs in the second individual discrete volumes each corresponding to a single cell of the population of cells.

2. The method of claim 1, wherein said RNA expression tracking construct comprises one or more sensing oligonucleotides and a targeting probe, wherein said targeting probe comprises a unique molecular identifier (UMI) and a 3′-terminal sequence capable of sequence-specific hybridization to a barcoding oligonucleotide.

3. The method of claim 2, further comprising the steps of:
(v) pooling the second individual discrete volumes, thereby forming a pooled mixture;
(vi) obtaining sequence information for the barcoded gene-specific amplicons and barcoded expression tracking constructs from the pooled mixture; and
(vii) identifying paired genotype information and target RNA expression levels from the sequence information by associating the UMI and barcode sequences of the single cell of the population of cells with the single cell,
thereby obtaining paired genotype information and target RNA expression levels from the single cell of the population of cells.

4. The method of claim 3, wherein in step (i) the first selection of oligonucleotides comprises two targeting probes capable of hybridizing the target RNA,
whereby the targeting probes bind to a target region in the target RNA and together form an initiator sequence when both targeting probes are bound to the target RNA sequence,
wherein the initiator sequence is not hybridized to the target region, and
wherein the first targeting probe comprises the unique molecular identifier (UMI) and the 3′-terminal sequence capable of sequence-specific hybridization to the barcoding oligonucleotide.

5. The method of claim 4, wherein step (i) further comprises:
providing a first sensing oligonucleotide, wherein the first sensing oligonucleotide is a hairpin that binds to the initiator sequence, wherein the hairpin is opened by hybridization to the initiator to reveal a hybridization region;
providing a second sensing oligonucleotide comprising a sequencing adaptor, wherein the second sensing oligonucleotide binds to the first sensing oligonucleotide via the hybridization region in the first sensing oligonucleotide; and
attaching the second sensing oligonucleotide to the first targeting probe via ligation, thereby generating the RNA expression tracking construct comprising the second sensing oligonucleotide and the first targeting probe comprising the UMI and the 3′-terminal sequence capable of sequence-specific hybridization to the barcoding oligonucleotide.

6. The method of claim 1, wherein the first individual discrete volumes, the second individual discrete volumes, or both, are droplets.

7. The method of claim 3, wherein the step of obtaining sequence information for the barcoded gene-specific amplicons and barcoded expression tracking constructs from the pooled mixture comprises:
performance of size selection to enrich for the gene-specific amplicons and/or performance of affinity selection to enrich for the RNA expression tracking constructs.

8. The method of claim 7, wherein the size selection to enrich for the gene-specific amplicons is by SPRI and/or wherein the affinity selection to enrich for the RNA expression tracking constructs comprises tagging the RNA expression tracking constructs with a biotin-labeled probe and binding the tagged RNA expression tracking constructs to a streptavidin-coated solid support.

9. The method of claim 1, wherein the bead-attached barcoding oligonucleotides are UV-detachable and/or the population of cells is fixed with methanol.

10. A method for obtaining paired target RNA expression levels and genotype information from a single cell of a population of cells, the method comprising:
(i) contacting a population of cells with a first selection of oligonucleotides capable of detecting target RNA levels, the first selection of oligonucleotides comprising two targeting probes capable of hybridizing the target RNA, whereby the targeting probes bind to a target region in the target RNA and together form an initiator sequence when both targeting probes are bound to the target RNA sequence, wherein the initiator sequence is not hybridized to the target region, wherein the first targeting probe comprises a unique molecular identifier (UMI) and a 3′-terminal sequence capable of sequence-specific hybridization to a barcoding oligonucleotide;
(ii) providing a first sensing oligonucleotide, wherein the first sensing oligonucleotide is a hairpin that binds to the initiator sequence, wherein the hairpin is opened by hybridization to the initiator to reveal a hybridization region;
providing a second sensing oligonucleotide comprising a sequencing adaptor, wherein the second sensing oligonucleotide binds to the first sensing oligonucleotide via the hybridization region in the first sensing oligonucleotide; and
attaching the second sensing oligonucleotide to the first targeting probe via ligation, thereby generating an RNA expression tracking construct, said RNA expression tracking construct comprising the second sensing oligonucleotide and the first targeting probe, said first targeting probe comprising the UMI and the 3′-terminal sequence capable of sequence-specific hybridization to a barcoding oligonucleotide;
(iii) encapsulating individual cells of the population of cells in first individual discrete volumes, said first individual discrete volumes comprising cell lysis buffer and proteases, thereby generating lysed individual cells in the first individual discrete volumes;
(iv) contacting the lysed individual cells in the first individual discrete volumes with a barcoding mixture comprising bead-attached barcoding oligonucleotides and genotyping probes under conditions sufficient to generate a population of lysed individual cells and individual beads comprising bead-attached barcoding oligonucleotides and genotyping probes in second individual discrete volumes;
(v) incubating the population of lysed individual cells and individual beads comprising bead-attached barcoding oligonucleotides and genotyping probes in the second individual discrete volumes under conditions sufficient to allow for:
amplification of the genotyping probes to proceed, thereby generating gene-specific amplicons in the second individual discrete volumes; and
polymerase-mediated extension of the bead-attached barcoding oligonucleotides along the gene-specific amplicons and the RNA expression tracking constructs in the second individual discrete volumes, thereby generating barcoded gene-specific amplicons and barcoded RNA expression tracking constructs in the second individual discrete volumes;
(vi) pooling the second individual discrete volumes, thereby forming a pooled mixture;
(vii) obtaining sequence information for the barcoded gene-specific amplicons and barcoded expression tracking constructs from the pooled mixture; and
(viii) identifying paired genotype information and target RNA expression levels from the sequence information by associating the UMI and barcode sequences of the single cell of the population of cells,
thereby obtaining paired genotype information and target RNA expression levels from the single cell of the population of cells.

11. A population of droplets, wherein for a majority of the droplets in the population of droplets, each droplet comprises:
a lysed individual cell;
one or more RNA expression tracking construct(s) comprising one or more sensing oligonucleotides and a targeting probe comprising a unique molecular identifier (UMI) and a 3′-terminal sequence capable of sequence-specific hybridization to a barcoding oligonucleotide;
a barcoding mixture comprising bead-attached barcoding oligonucleotides; and
genotyping probes.

12. The population of droplets of claim 11, wherein for a majority of the droplets in the population of droplets, each droplet further comprises gene-specific amplicons produced via amplification of the genotyping probes.

13. The population of droplets of claim 12, wherein for a majority of the droplets in the population of droplets, each droplet further comprises barcoded gene-specific amplicons and barcoded RNA expression tracking constructs produced via polymerase-mediated extension of the bead-attached barcoding oligonucleotides along the gene-specific amplicons and the RNA expression tracking constructs.

14. A method for obtaining paired target RNA expression levels and genotype information from a single cell of a population of cells, the method comprising:
pooling the population of droplets of claim 13, thereby forming a pooled mixture;
obtaining sequence information for the barcoded gene-specific amplicons and barcoded expression tracking constructs from the pooled mixture; and
identifying paired genotype information and target RNA expression levels from the sequence information by associating the UMI and barcode sequences of the single cell of the population of cells,
thereby obtaining paired genotype information and target RNA expression levels from the single cell of the population of cells.

15. The method of claim 14, wherein the step of obtaining sequence information for the barcoded gene-specific amplicons and barcoded expression tracking constructs from the pooled mixture comprises: performance of size selection to enrich for the gene-specific amplicons and/or performance of affinity selection to enrich for the RNA expression tracking constructs.

16. The method of claim 15, wherein the size selection to enrich for the gene-specific amplicons is by SPRI.

17. The method of claim 15, wherein the affinity selection to enrich for the RNA expression tracking constructs comprises tagging the RNA expression tracking constructs with a biotin-labeled probe and binding the tagged RNA expression tracking constructs to a streptavidin-coated solid support.

18. The method of claim 14, wherein the bead-attached barcoding oligonucleotides are UV-detachable.

19. A method for identifying a genomic variant that possesses functional impact in a cell to which the genomic variant is introduced, the method comprising:
(a) introducing the genomic variant to the genome of the cell;
(b) obtaining, by the method of claim 3, paired target RNA expression levels and genotype information for the genomic variant from single cells of a population of cells, wherein the single cells are confirmed by the genotype information to possess the genomic variant; and
(c) identifying altered target RNA expression levels in single cells confirmed by the genotype information to possess the genomic variant,
thereby identifying a genomic variant that possesses functional impact in the cell to which the genomic variant is introduced.

20. The method of claim 19, wherein the genomic variant is introduced via use of a CRISPR/Cas system.
